SEQUENCE LISTING (8 sequences)

SEQ ID NO: 1 - protein, 5 aa, artificial sequence
  synthetic peptide sequence
GSGGS

SEQ ID NO: 2 - protein, 4 aa, artificial sequence
  synthetic peptide sequence
GGGS

SEQ ID NO: 3 - protein, 4 aa, artificial sequence
  synthetic peptide sequence
GGSG

SEQ ID NO: 4 - protein, 5 aa, artificial sequence
  synthetic peptide sequence
GGSGG

SEQ ID NO: 5 - protein, 5 aa, artificial sequence
  synthetic peptide sequence
GSGSG

SEQ ID NO: 6 - protein, 5 aa, artificial sequence
  synthetic peptide sequence
GSGGG

SEQ ID NO: 7 - protein, 5 aa, artificial sequence
  synthetic peptide sequence
GGGSG

SEQ ID NO: 8 - protein, 5 aa, artificial sequence
  synthetic peptide sequence
GSSSG

(19) United States
(12) Patent Application Publication
(10) Pub. No.: US 2022/0119466 A1
(43) Pub. Date: Apr. 21, 2022

(54) SYNTHETIC MOLECULAR FEEDBACK CIRCUITS AND METHODS OF USING THE SAME

(71) Applicant: The Regents of the University of California, Oakland, CA (US)

(72) Inventors: Hana El-Samad, San Francisco, CA (US); Andrew Ng, San Francisco, CA (US)

(21) Appl. No.: 17/419,234

(22) PCT Filed: Jan. 6, 2020
(86) PCT No.: PCT/US2020/012353

§ 371 Date: Jun. 28, 2021

Related U.S. Application Data
(60) Provisional application No. 62/789,402, filed on Jan. 7, 2019.

(51) Int. Cl. C07K 14/47; C12N 9/50; A61K 35/17

(52) U.S. Cl. CPC C07K 14/4705; C07K 2319/095; A61K 35/17; C12N 9/50

(57) ABSTRACT

Provided are molecular feedback circuits as well as nucleic acids encoding such molecular feedback circuits and cells genetically modified with the subject molecular feedback circuits. Methods of modulating signaling of a signaling pathway of a cell using molecular feedback circuits and methods of treating a subject for a condition by administering a cell containing a nucleic acid that encodes a molecular feedback circuit are also provided. Aspects of the molecular feedback circuits of the present disclosure include a signaling protein, of a signaling pathway, that includes a latent deactivation domain. Such circuits may include a regulatory sequence that is responsive to an output of the signaling pathway and is operably linked to a nucleic acid encoding a switch polypeptide that, when expressed, triggers the deactivation domain to deactivate the signaling molecule.

### CROSS-REFERENCING

This application claims the benefit of U.S. provisional application Ser. No. 62/789,402, filed on Jan. 7, 2019, which application is incorporated by reference herein.

### STATEMENT REGARDING FEDERALLY SPONSORED RESEARCH

This invention was made with government support under Grant No. HR0011-16-2-0045 awarded by the Defense Advanced Research Projects Agency. The government has certain rights in the invention.

### INTRODUCTION

Conventionally, desired regulation of cellular activities has been controlled by repeated, user-provided inputs to cellular systems. For example, in the context of some medical treatments, a desired level of a cellular output in a subject over an extended period of time is achieved by repeated cycles of dosing an agent, assessing, re-dosing and re-assessing over the course of treatment. Similarly, in bioproduction applications and metabolic engineering, to coax production cells to output desired yields of product, growth media is repeatedly augmented, e.g., by supplementing growth factors and/or removing toxic byproducts.

Huge advances in the abilities of engineered cells to perform desired tasks, and methods for producing such engineered cells, have been made in recent decades. For example, recent progress in synthetic biology and systems metabolic engineering technologies provide the potential of microbial cell factories for the production of industrially relevant bulk and fine chemicals from renewable biomass resources in an eco-friendly manner. In addition, designer cell therapies, such as chimeric antigen receptor (CAR) T cell therapies, which may be directed to various user-defined targets, have shown great promise in the clinic and are gaining wide adoption and continued regulatory approval.

Without further user-input, the output of such engineered cells, e.g., as used for various purposes as described above, is constant once administered to a subject or set in motion in a bioreactor. Adjustments to modulate engineered cell output are made using an external input, e.g., in the form of small molecules, or other stimuli or user-performed actions.

### SUMMARY

Provided are molecular feedback circuits as well as nucleic acids encoding such molecular feedback circuits and cells genetically modified with the subject molecular feedback circuits. Methods of modulating signaling of a signaling pathway of a cell using molecular feedback circuits and methods of treating a subject for a condition by administering a cell containing a nucleic acid that encodes a molecular feedback circuit are also provided. Aspects of the molecular feedback circuits of the present disclosure include a signaling protein, of a signaling pathway, that includes a latent deactivation domain. Such circuits may include a regulatory sequence that is responsive to an output of the signaling pathway and is operably linked to a nucleic acid encoding a switch polypeptide that, when expressed, triggers the deactivation domain to deactivate the signaling molecule.

In any embodiment, the signaling protein does not comprise a caged degron. In other words, in any embodiment, the signaling protein does not comprise: (a) a degron. (b) a locker domain comprising five alpha helices, and (c) a latch domain comprising an alpha helix that: (i) in the absence of a key polypeptide, forms a six helix bundle with the locker domain to cage the degron and prevent degradation of the signaling protein and (ii) in the presence of the key polypeptide, the degron is uncaged and the signaling protein is degraded, where the key polypeptide can comprise an alpha helix that binds the locker domain with higher affinity than the latch domain. Such caged degron molecules are described in provisional application Ser. Nos. 62/789,418 (filed on Jan. 7, 2019), 62/850,336 (filed on May 20, 2019) and 62/789,351, filed on Jan. 7, 2019, which applications are incorporated by reference herein.

### BRIEF DESCRIPTION OF THE DRAWINGS

FIG. 1 schematically depicts a signaling pathway as described herein.

FIG. 2 schematically depicts a signaling pathway with a latent deactivation domain attached to a positive regulatory member of the signaling pathway of FIG. 1 as described herein.

FIG. 3 depicts the activation of the latent deactivation domain in the schematically depicted signaling pathway of FIG. 2 as described herein.

FIG. 4 schematically depicts a signaling pathway with a latent deactivation domain attached to a negative regulatory member of the signaling pathway of FIG. 1 as described herein.

FIG. 5 schematically depicts a molecular feedback circuit strategy employing a synthetic Notch receptor as described herein.

FIG. 6 schematically depicts a molecular feedback circuit strategy employing a chimeric antigen receptor (CAR) as described herein.

FIG. 7 schematically depicts various strategies for controlling feedback in examples of feedback circuits described in the present disclosure.

FIG. 8 schematically depicts an example of a sequestration-based strategy for controlling feedback in a circuit described herein.

FIG. 9 provides an alternative depiction of an example of a sequestration-based strategy for controlling feedback in a circuit described herein.

FIG. 10 demonstrates feedback control using an example of a sequestration-based feedback circuit of the present disclosure.

FIG. 11 schematically depicts a leucine zipper transcription factor and a dominant negative inhibitor of the leucine zipper transcription factor.

FIG. 12 schematically depicts an example of a competition-based strategy for controlling feedback in a circuit described herein.

FIG. 13 demonstrates feedback control using an example of a competition-based feedback circuit of the present disclosure.

FIG. 14 provides a schematic depiction of a degronLOCKR-based feedback circuit or controlling biological pathways.

FIG. 15 provides a panel of mating pathway regulators tested with degronLOCKR.

FIG. 16 demonstrates that degronLOCKR module successfully implements synthetic feedback control of the mating pathway.

FIG. 17 provides operational properties of degronLOCKR feedback module quantified via control of a synthetic circuit.

FIG. 18 provides steady state solutions in response to positive or negative disturbances.

FIG. 19 depicts circuit behavior as a function of Pg for a fixed dose of E2.

FIG. 20 depicts circuit behavior as a function of E2 for a fixed dose of Pg.

FIG. 21 depicts circuit behavior when expressing different amounts of key constitutively.

FIG. 22 demonstrates that the DegronLOCKR synthetic feedback strategy is predictably tunable.

FIG. 23 demonstrates that changing promoter strength or key length modulates feedback gain.

FIG. 24 demonstrates that tuning feedback strength changes dynamic behavior of circuit output.

FIG. 25 depicts combinatorial tuning of synthetic feedback in mating pathway.

FIG. 26 depicts control of protein localization using nesLOCKR.

FIG. 27 depicts cytosolic aggregation of nesLOCKR when Key is expressed.

FIG. 28 shows fluorescence histograms of tagBFP (left panel) and fluorescence histograms of mCherry (right panel).

FIG. 29 shows no feedback and feedback circuit diagrams (top panels) and representative histograms comparing output and key fluorescence for both circuits in the presence and absence of drug (bottom panels).

FIG. 30 shows a comparison of output for different feedback variants (left panel) and a normalized output for circuit with no feedback and feedback circuit with mCMV-Key (right panel).

### DEFINITIONS

The terms “synthetic”, “chimeric” and “engineered” as used herein generally refer to artificially derived polypeptides or polypeptide encoding nucleic acids that are not naturally occurring. Synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods. Chimeric and engineered polypeptides or polypeptide encoding nucleic acids will generally be constructed by the combination, joining or fusing of two or more different polypeptides or polypeptide encoding nucleic acids or polypeptide domains or polypeptide domain encoding nucleic acids. Chimeric and engineered polypeptides or polypeptide encoding nucleic acids include where two or more polypeptide or nucleic acid “parts” that are joined are derived from different proteins (or nucleic acids that encode different proteins) as well as where the joined parts include different regions of the same protein (or nucleic acid encoding a protein) but the parts are joined in a way that does not occur naturally.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell. a nucleic acid, a protein, or a vector) that the material has been modified by the introduction of a heterologous material (e.g., a cell, a nucleic acid, a protein, or a vector).

The term “operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

A “biological sample” encompasses a variety of sample types obtained from an individual or a population of individuals and can be used in various ways, including e.g., the isolation of cells or biological molecules, diagnostic assays, etc. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by mixing or pooling of individual samples, treatment with reagents, solubilization, or enrichment for certain components, such as cells, polynucleotides, polypeptides. etc. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples. Accordingly, biological samples may be cellular samples or acellular samples.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

A “vector” or “expression vector” is a replicon, such as plasmid, phage, virus, or cosmid, to which another DNA segment, i.e. an “insert”, may be attached so as to bring about the replication of the attached segment in a cell.

The terms “domain” and “motif”, used interchangeably herein, refer to both structured domains having one or more particular functions and unstructured segments of a polypeptide that, although unstructured, retain one or more particular functions. For example, a structured domain may encompass but is not limited to a continuous or discontinuous plurality of amino acids, or portions thereof, in a folded polypeptide that comprise a three-dimensional structure which contributes to a particular function of the polypeptide. In other instances, a domain may include an unstructured segment of a polypeptide comprising a plurality of two or more amino acids, or portions thereof, that maintains a particular function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6:197-208.

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more.

The term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁻⁷ M, e.g., binding with an affinity of 10⁻⁶ M, 10⁻⁵ M, 10⁻⁴ M, etc.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled (e.g., as described in PCT publication no. WO 2014/127261 A1 and US Patent Application No. 2015/0368342 A1, the disclosures of which are incorporated herein by reference in their entirety). CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. Useful CARs also include the anti-CD19-4-1BB-CD3ζ CAR expressed by lentivirus loaded CTL019 (Tisagenlecleucel-T) CAR-T cells as commercialized by Novartis (Basel, Switzerland). The terms “chimeric antigen receptor” and “CAR” also include SUPRA CAR and PNE CAR (see, e.g., Cho et al Cell 2018 173: 1426-1438 and Rodgers et al, Proc. Acad. Sci. 2016 113: E459-468).

The terms “T cell receptor” and “TCR” are used interchangeably and will generally refer to a molecule found on the surface of T cells, or T lymphocytes, that is responsible for recognizing fragments of antigen as peptides bound to major histocompatibility complex (MHC) molecules. The TCR complex is a disulfide-linked membrane-anchored heterodimeric protein normally consisting of the highly variable alpha (α) and beta (β) chains expressed as part of a complex with CD3 chain molecules. Many native TCRs exist in heterodimeric αβ or γδ forms. The complete endogenous TCR complex in heterodimeric αβ form includes eight chains, namely an alpha chain (referred to herein as TCRα or TCR alpha), beta chain (referred to herein as TCRβ or TCR beta), delta chain, gamma chain, two epsilon chains and two zeta chains. In some instance, a TCR is generally referred to by reference to only the TCRα and TCRβ chains, however, as the assembled TCR complex may associate with endogenous delta, gamma, epsilon and/or zeta chains an ordinary skilled artisan will readily understand that reference to a TCR as present in a cell membrane may include reference to the fully or partially assembled TCR complex as appropriate.

Recombinant or engineered individual TCR chains and TCR complexes have been developed. References to the use of a TCR in a therapeutic context may refer to individual recombinant TCR chains. As such, engineered TCRs may include individual modified TCRα or modified TCRβ chains as well as single chain TCRs that include modified and/or unmodified TCRα and TCRβ chains that are joined into a single polypeptide by way of a linking polypeptide.

The terms “synthetic Notch receptor”, “synNotch” and “synNotch receptor”, used interchangeably herein, refer to recombinant chimeric binding-triggered transcriptional switches that include at least: an extracellular binding domain, a portion of a Notch receptor that includes at least one proteolytic cleavage site, and an intracellular domain that provides a signaling function. SynNotch polypeptides, the components thereof and methods of employing the same, are described in U.S. Pat. Nos. 9,834,608 and 9,670,281, as well as, Toda et al., Science (2018) 361(6398):156-16; Roybal & Lim, Annu Rev Immunol. (2017) 35:229-253; Lim & June Cell. (2017) 168(4):724-740; Roybal et al. Cell. (2016) 167(2):419-432.e16; Roybal et al. Cell. (2016) 164(4):770-9; and Morsut et al. Cell. (2016) 164(4):780-91; the disclosures of which are incorporated herein by reference in their entirety.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a circuit” includes a plurality of such circuits and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

Provided are molecular feedback circuits as well as nucleic acids encoding such molecular feedback circuits and cells genetically modified with the subject molecular feedback circuits. Methods of modulating signaling of a signaling pathway of a cell using molecular feedback circuits and methods of treating a subject for a condition by administering a cell containing a nucleic acid that encodes a molecular feedback circuit are also provided. Aspects of the molecular feedback circuits of the present disclosure include a signaling protein, of a signaling pathway, that includes a latent deactivation domain. Such circuits may include a regulatory sequence that is responsive to an output of the signaling pathway and is operably linked to a nucleic acid encoding a switch polypeptide that, when expressed, triggers the deactivation domain to deactivate the signaling molecule.

Molecular Circuits

Molecular circuits of the present disclosure may, in some instances and in whole or in part, be encoded by nucleic acid sequences. Such circuits may, in some instances, be present and/or configured in expression vectors and/or expression cassettes. The subject nucleic acids of the present circuits may, in some instances, be contained within a vector, including e.g., viral and non-viral vectors. Such circuits may, in some instances, be present in cells, such as immune cells, stem cells, etc., or may be introduced into cells by various means, including e.g., through the use of a viral vector. Cells may, in some instances, be genetically modified to contain and/or encode a subject circuit, where such modification may be effectively permanent (e.g., integrated) or transient as desired.

Circuits of the present disclosure, the components of which are modular, may include a signaling protein that includes a latent deactivation domain. As used herein, the term “signaling protein” generally refers to a protein of a signaling pathway, including natural and synthetic signaling pathways, described in more detail below. Any convenient and appropriate signaling protein of any convenient signaling pathway may be employed. Generally, signaling proteins include proteins that may be activated by an input of the signaling pathway with which the signaling protein is associated. A signaling pathway may generate an output that is dependent upon, or at least influenced by, the function of the signaling protein. Such outputs may be a direct or indirect result of the response of the signaling protein to the input. Useful signaling proteins include members from any convenient and appropriate point in a signaling pathway, including input-receiving members, intermediate members, and output-producing members.

By “input-receiving members”, as used herein, is generally meant the initial component of a signaling pathway that receives an input to initiate signaling along the pathway. Examples of input-receiving members include but are not limited to e.g., extracellular receptors (e.g., G protein-coupled receptors, protein kinases, integrins, toll-like receptors, ligand-gated ion channels, and the like) and intracellular receptors (e.g., nuclear receptors, cytoplasmic receptors, etc.). In some instances, an input-receiving member may be a protein that directly binds an input of a signaling pathway, such as a ligand input of a signaling pathway. In some instances, a signaling protein that includes a latent deactivation domain in a circuit of the present disclosure may be an input-receiving member. In some instances, a signaling protein that includes a latent deactivation domain in a circuit of the present disclosure may not be an input-receiving member, e.g., it may be an intermediate member or an output-producing member.

By “intermediate member”, as used herein, is generally meant a component of a signaling pathway that is required for, or at least involved in, signal transduction but does not directly receive the initial input or directly produce or cause the final output of the signaling pathway. Examples of intermediate members of a signaling pathway include but are not limited to e.g., enzymes, binding partners, protein complex subunits, scaffold proteins, transport proteins, co-activators, co-repressors, and the like. In some instances, a signaling protein that includes a latent deactivation domain in a circuit of the present disclosure may be an intermediate member. In some instances, a signaling protein that includes a latent deactivation domain in a circuit of the present disclosure may not be an intermediate member, e.g., it may be an input-receiving member or an output-producing member.

By “output-producing member”, as used herein, is generally meant a component of a signaling pathway that directly produces an output of the signaling pathway or otherwise causes the output of the signaling pathway to occur. Examples of output-producing members of a signaling pathway include but are not limited to e.g., DNA binding proteins, such as e.g., transcription factors, enzymes, and the like. In some instances, a signaling protein that includes a latent deactivation domain in a circuit of the present disclosure may be an output-producing member. In some instances, a signaling protein that includes latent deactivation domain in a circuit of the present disclosure may not be an output-producing member, e.g., it may be an input-receiving member or an intermediate member.

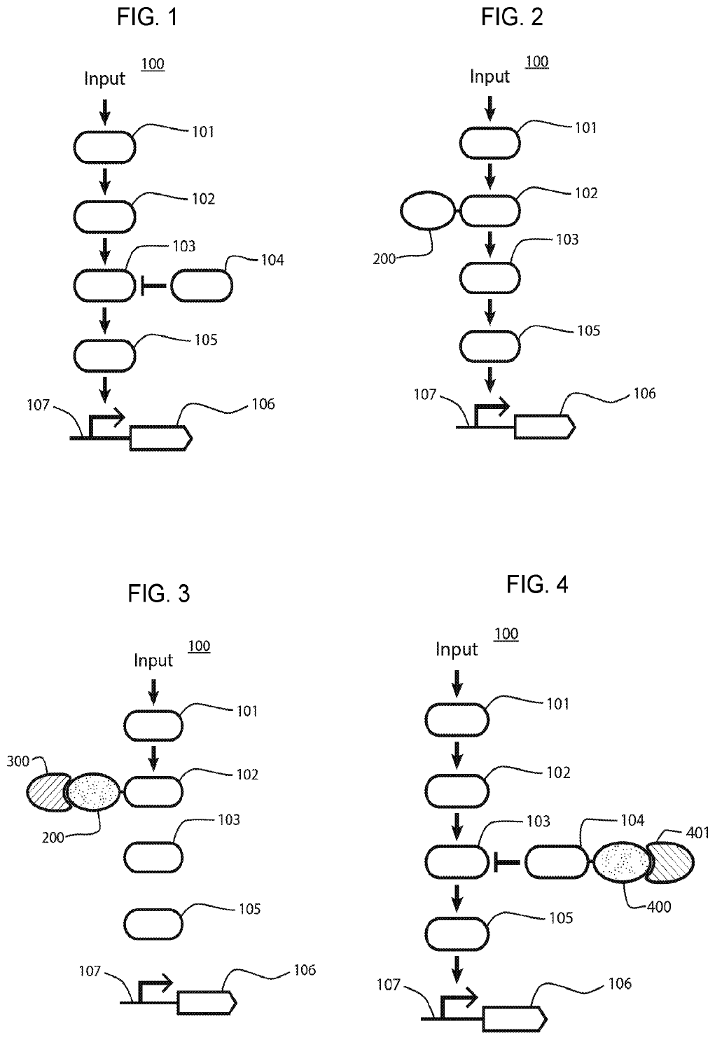

A schematized example of a signaling pathway is depicted in FIG. 1. As shown, the signaling pathway includes an input 100 that activates an input-receiving member 101 of the pathway. Activation of the input-receiving member 101 positively regulates a first intermediate member 102 of the pathway, which positively regulates a second intermediate member 103 of the pathway. In the pathway depicted, the second intermediate member 103 is negatively regulated by a third intermediate member 104. In the absence of inhibition by the third intermediate member 104, the second intermediate member 103 positively regulates an output-producing member 105 of the pathway. Thus, in the presence of activation by the second intermediate member 103, the output-producing member 105 is active and binds a regulatory region 107 operably linked to a sequence 106 encoding an output of the signaling pathway.

Useful signaling proteins may be a regulator of one or more signaling pathways with which the signaling protein is associated, including where the signaling protein may be a negative regulator of a signaling pathway or a positive regulator of a signaling pathway. Accordingly, molecular feedback circuits of the present disclosure include positive feedback circuits and negative feedback circuits.

For example, in some instances, a signaling protein employed in a circuit of the present disclosure may, when activated, drive an output of the signaling pathway. As such, triggering a latent deactivation domain to deactivate the signaling protein, may negatively regulate the output of the signaling pathway thus resulting in negative feedback. In some instances, a signaling protein employed in a circuit of the present disclosure may, when activated, inhibit an output of the signaling pathway. As such, triggering a latent deactivation domain to deactivate the signaling protein, may positively regulate the output of the signaling pathway thus resulting in positive feedback.

FIG. 2 depicts the signaling pathway presented in FIG. 1 where a first intermediate signaling member 102, that positively regulates the pathway, has been modified to include a latent deactivation domain 200. Thus, when the latent deactivation domain 200 remains in a latent state, signaling through the signaling pathway proceeds from the input 100, through the input-receiving member 101, to the first intermediate signaling member 102, which positively regulates downstream components of the pathway, such that, in the absence of inhibition by the third intermediate member (not pictured), the output-producing member 105 drives the output of the signaling pathway, depicted as expression of the product encoded by the sequence 106.

As depicted in FIG. 3, an expressed switch polypeptide 300 activates the latent deactivation domain 200 resulting in deactivation of the first intermediate member 102 and, thus a lack of positive signaling from the first intermediate member 102 to the second intermediate member 103 and subsequent points of the pathway. Accordingly, output from the sequence 106 is not generated or is reduced.

In another example, depicted in FIG. 4, a latent deactivation domain 400 is attached to the inhibitory third intermediate member 104. Thus, when the latent deactivation domain 400 remains in a latent state, the presence of the third intermediate member 104 negatively regulates the second intermediate member thereby repressing expression and production of the product encoded by the output sequence 106. Correspondingly, when the latent deactivation domain 400 is activated by an expressed switch polypeptide 401, the third intermediate member 104 is deactivated, thereby preventing negative regulation by the third intermediate member 104 and positively regulating the pathway 100 to promote generation of the output, i.e., at least an increase in expression of the product encoded by the sequence 106.

When integrated with a switch polypeptide, the expression of which is driven by an output of the signaling pathway, coupling a latent deactivation domain to a signaling protein of the pathway may provide for positive or negative feedback as desired. For example, coupling the latent deactivation domain to positive signaling regulators creates negative feedback on the pathway, whereas coupling the latent deactivation domain to negative regulators creates positive feedback on the pathway. In some instances, negative pathway feedback can be used to dampen responses, whereas, in some instances, positive pathway feedback can be used to amplify responses or generate ultra-sensitivity. As will be readily evident, the feedback circuits of the present disclosure are highly modular and thus, circuits described herein may be readily modified as desired and/or applied to essentially any convenient and appropriate signaling pathway, including e.g., signaling pathways with measurable output via a promoter.

Feedback control enables robust, stable performance of a physical process through disturbance rejection. Implementation of feedback control may generally include: (1) the ability to measure or “sense” the output of the process, (2) a controller to generate a corrective signal based on a comparison of the output measurement against a desired output or “setpoint”, and (3) a method to input or “actuate” the corrective signal to the process to be controlled. Provided herein are designed circuits that utilize latent deactivation domain-based protein switches, triggered by expressed switch polypeptides, to generate feedback control of biological systems. Specifically, three modules analogous to the ones described above: (1) a sensing promoter that is activated by the output of the process of interest, (2) a switch peptide produced by the sensing promoter that activates degradation of (3) a signaling protein (i.e., transcription factor, kinase, etc.) that is fused to a latent deactivation domain. Each of these modules can be independently tuned, as desired, via simple manipulations to achieve the desired feedback control of the process.

Signaling proteins that may be employed in the circuits of the present disclosure include signaling proteins that are endogenous components of the signaling pathway as well as heterologous or synthetic components of the signaling pathway. Such endogenous, heterologous and/or synthetic components of signaling pathways may be modified to include a latent deactivation domain, described in more detail below, for use in a circuit of the present disclosure. By “endogenous component of the signaling pathway” is generally meant a component of the signaling pathway as it occurs naturally in a cell.

By “heterologous component of the signaling pathway” is generally meant a component that functions in the signaling pathway but is derived from a cell or signaling pathway other than that in which it is employed in the subject circuit. Heterologous components may be derived from a signaling pathway separate from the signaling pathway of the subject circuit. Heterologous components may be derived from a different type of cell and/or a different organism from the cell and/or organism of the signaling pathway modulated in the subject circuit. For example, in some instances, a component of a signaling pathway from a first organism (e.g., mouse) may be employed in a corresponding signaling pathway of a second organism (e.g., human).

By “synthetic component of the signaling pathway” is generally meant a component that functions in the signaling pathway but is non-naturally derived. Non-naturally derived components may include recombinant components, including e.g., analogs, mimetics, fusions, mutants, truncated versions, fragments, and the like. Non-limiting examples of synthetic components of signaling pathways including synthetic receptors, synthetic enzymes, synthetic co-activators, synthetic co-repressors, synthetic binding partners, synthetic scaffold proteins, synthetic transcription factors, and the like.

Circuits of the present disclosure may employ one or more regulatory sequences, the control of which may be dependent upon a component of the signaling pathway with which the signaling protein is associated. For example, in some instances, a circuit of the present disclosure may include a regulatory sequence responsive to an output of the signaling pathway. Regulatory sequences may be operably linked to one or more nucleic acid sequences encoding one or more components of the subject circuit. For example, a regulatory sequence may be operably linked to a nucleic acid sequence encoding a switch polypeptide.

In some instances, a circuit may include a regulatory sequence operably linked to a nucleic acid sequence encoding the signaling protein. Regulatory sequences operably linked to a sequence encoding the signaling protein of the subject circuits may vary and may include endogenous and heterologous regulatory sequences, including but not limited to e.g., native promoters, native enhancers, heterologous promoters, heterologous enhancers, synthetic regulatory sequences, and the like. Regulatory sequences operably linked to a sequence encoding the signaling protein may be constitutive or inducible as desired. In some instances, a regulatory sequence operably linked to the nucleic acid sequence encoding a signaling protein is a native promoter of the signaling protein.

In some instances, a regulatory sequence may include one or more binding sites (e.g., 1 or more. 2 or more, 2 to 10, 3 to 10, 4 to 10, 5 to 10, 2 to 6, 3 to 6, 4 to 6, 1, 2, 3, 4, 5, 6, 7, 8, 9, 10, etc.) for a transcription factor of the output, including e.g., where the transcription factor is an endogenous, heterologous, or synthetic transcription factor that functions in the signaling pathway.

Regulatory sequences of circuits of the present disclosure may be controlled by, or otherwise responsive to, an output of a signaling pathway. For example, in some instances, an output of a signaling pathway, which the subject circuit is configured to influence, may induce expression of a coding sequence through a regulatory sequence operably linked to the coding sequence. By connecting the regulation of a sequence encoding a component of a circuit of the present disclosure to an output of the signaling pathway, circuits of the present disclosure may provide feedback that is response to the output.

Useful signaling pathway outputs employing in circuits of the present disclosure may vary and may include essentially any output that may be configured to directly or indirectly influence expression through a regulatory sequence. Non-limiting examples of useful signaling pathway outputs include but are not limited to e.g., activity (e.g., activation, repression, etc.) of a transcription factor, expression of a transcription factor, translocation of a transcription factor, activity (e.g., activation, repression, etc.) of an enzyme, expression of an enzyme, production of a signaling molecule, secretion of a signaling molecule, cellular activation (including e.g., activation of native cellular programs, such as but not limited to e.g., immune activation, immune suppression, proliferation, etc.), and the like.

Signaling pathways may be modulated (e.g., activated, repressed, etc.) by one or more inputs. Inputs of signaling pathways may vary and may include endogenous (e.g., native) inputs of signaling pathways and heterologous (e.g., engineered or synthetic) signaling pathway inputs. As signaling pathways, and signaling pathway outputs, may be native or synthetic, signaling pathway inputs may similarly be native or synthetic.

Native signaling pathways may, in many instances, be controlled by a native or natural receptor of the pathway. Non-limiting examples of native signaling pathways include but are not limited to e.g., the AKT signaling pathway, the Akt/PKB signaling pathway, the AMPK signaling pathway, the apoptosis signaling pathway, the BMP signaling pathway, the cAMP-dependent pathway, the estrogen signaling pathway, the hedgehog signaling pathway, the hippo signaling pathway, an immune activation pathway, an immune suppression pathway, an immune cell differentiation pathway, an insulin signal transduction pathway, the JAK-STAT signaling pathway, the MAPK/ERK signaling pathway, the mTOR signaling pathway, the NF-κB signaling pathway, the nodal signaling pathway, the notch signaling pathway, the p53 signaling pathway, the PI3K signaling pathway, the TGF beta signaling pathway, the TLR signaling pathway, the TNF signaling pathway, the VEGF signaling pathway, the Wnt signaling pathway, and the like.

Non-limiting examples of synthetic signaling pathways include, but are not limited to, those pathways controlled by a synthetic or engineered receptor, such as but not limited to e.g., a CAR, an engineered TCR, a synNotch, etc. Signaling pathways are described in more detail below.

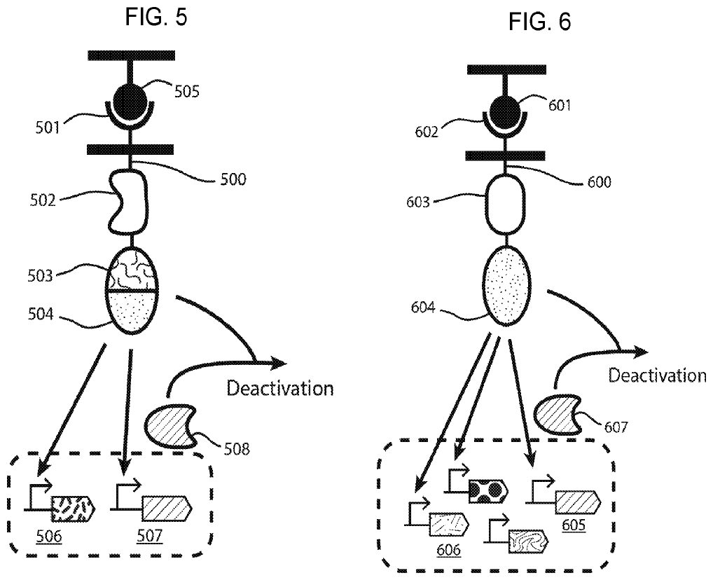

Schematized examples of modulating a synthetic synNotch signaling pathway and a synthetic CAR signaling pathway using circuits of the present disclosure are depicted in FIG. 5 and FIG. 6, respectively. As shown in FIG. 5, a synNotch receptor 500, having an antigen binding domain 501, a proteolytically cleavable Notch domain 502, and an intracellular signaling domain, that includes a synthetic transcription factor (synTF) portion 503 and a latent deactivation domain 504, is triggered by an antigen input 505 to release the intracellular signaling domain. Release of the intracellular signaling domain of the synNotch after antigen binding induces expression of a desired output 506 which is controlled by the synTF. In the embodiment pictured, the synTF output also controls expression 507 of a switch polypeptide 508. Thus, when the released intracellular signaling domain of the synNotch induces expression of the switch polypeptide, the switch polypeptide 508 activates the latent deactivation domain 504 to deactivate the synTF-containing intracellular signaling domain of the synNotch receptor. Accordingly, by providing negative feedback through the synthetic synNotch signaling pathway a controlled custom output is generated.

As shown in FIG. 6, a CAR 600 is triggered by an antigen input 601 binding to an antigen binding domain 602 to induce an internal signaling cascade, e.g., leading immune cell activation through immune stimulatory signaling through the CD3z domain 603 of the CAR 600. The CAR 600 also includes an attached latent deactivation domain 604 and the cell includes a regulatory sequence, operably linked to a sequence 605 encoding a switch polypeptide that is responsive to a component of the internal signaling cascade. Thus, activation of the internal signaling cascade induces expression of a desired output 606, such as immune cell activation and/or expression of desired immune factors that is controlled by the CAR. However, in the embodiment pictured, the CAR output also controls expression of the switch polypeptide 607. Thus, when the signaling cascade induces expression of the switch polypeptide 607, the switch polypeptide 607 activates the latent deactivation domain 604, which deactivates the CAR. Accordingly, by providing negative feedback through the synthetic CAR signaling pathway T-cell activation is controlled.

As will be readily apparent, these examples employing synthetic signaling pathways are not intended to be limiting.

Latent Deactivation Domains and Switch Polypeptides

As summarized above, signaling proteins employed in the circuits of the present disclosure may include a latent deactivation domain. The latent deactivation domain included in a subject signaling protein of the present circuits may vary and may be attached or otherwise integrated into the signaling protein as desired. Any convenient method of attaching or integrating a latent deactivation domain into a subject signaling protein may be employed, including but not limited to e.g., where the latent deactivation domain is attached via a linker.

Latent deactivation domains, in the absence of a switch polypeptide, remain in a latent state, meaning the deactivation domain does not deactivate the signaling protein with which it is associated. In the presence of the switch polypeptide the latent deactivation domain is activated and subsequently deactivates the signaling proteins with which it is associated. Strategies for deactivation will vary and may include but are not limited to e.g., inducible degradation, inducible localization, protein splitting, and the like. Switch polypeptides may correspondingly vary and may include but are not limited to e.g., polypeptides that induce degradation of a latent deactivation domain, polypeptides that induce localization of a latent deactivation domain, polypeptides that induce splitting of a latent deactivation domain, and the like.

As summarized above, latent deactivation domains include inducible degradation domains. By “inducible degradation domain”, as used herein, is generally meant a domain capable modulating (e.g., enhancing, increasing, etc.) degradation of a polypeptide, such as a signaling protein, to which the inducible degradation domain is attached or becomes associated with, upon expression of a switch polypeptide. Inducible degradation domains will generally not modulate (e.g., enhance, increase, etc.) degradation of a subject signaling protein when the corresponding switch polypeptide is not expressed. Thus, the degradation domain will generally be latent until activated by expression of a switch polypeptide to induce degradation of a polypeptide to which it is attached or becomes associated with. Degradation of a polypeptide, such as a signaling protein, to which the degradation domain is attached or becomes associated with, deactivates the polypeptide.

Configurations of latent deactivation domains that include an inducible degradation domain will vary, as will the corresponding switch polypeptide. In some instances, a degradation domain may include a degron. In some instances, an inducible degradation domain may include a protection domain that prevents degradation of a polypeptide (e.g., a signaling protein) attached to the degradation domain. A degradation domain protected by a protection domain may be deprotected by a switch polypeptide, such that the degradation domain becomes active to trigger degradation of an attached, or otherwise associated, polypeptide.

For example, in some embodiments, a degradation domain, attached to a signaling protein, may be protected by a protection domain that includes a proteolytic cleavage site that is cleavable by a protease included in a switch polypeptide. In the absence of switch polypeptide, the protease is not present and the protection domain prevents the degradation domain from triggering degradation of signaling protein. In the presence of the switch polypeptide, the protease cleaves the proteolytic cleavage site, deprotecting the degradation domain and triggering degradation of the signaling protein.

In some embodiments, a switch polypeptide may include a protease, such as a tobacco etch virus (TEV) protease that will cleave either a N- or C-terminal sequence present on an inducible deactivation domain attached to a signaling protein. Cleavage by the protease reveals either a N- or C-terminal degron that then triggers degradation of the signaling protein. Accordingly, in the absence of the TEV protease-containing switch polypeptide the degron remains hidden and does not induce degradation of the signaling protein. In some instances, components and configurations that may be employed in a protease-inducible degradation domain system may include those employed in the CHOMP (circuits of hacked orthogonal modular proteases) system as described in Gao et al., Science. 2018; 361(6408):1252-1258; the disclosure of which is incorporated herein by reference in its entirety. As will be readily understood, in some instances, other degrons and/or other encoded proteases may be substituted for those explicitly described.

In some embodiments, induced degradation may be achieved by ligation of a degradation sequence to a target signaling protein. For example, in some instances, two peptides that bind to one another when both peptides are present may be employed to ligate a degradation sequence to a target signaling molecule in an inducible manner. Binding of the peptides to one another may, in some instances, be covalent. In some embodiments, a tag peptide that binds to a tag-binding domain may be incorporated into a target signaling protein and a switch protein may be configured to include a tag-binding domain with an attached degradation sequence, such as a constitutive degron. Upon expression of such a switch protein, the tag and tag-binding domain bind each other, thereby attaching the degradation sequence to the target signaling protein and inducing degradation of the signaling protein. As will be readily understood, in some instances, the tag and tag-binding domain may be swapped, i.e., a tag-binding domain that binds to a tag peptide may be incorporated into a target signaling protein and a switch protein may be configured to include a tag peptide with an attached degradation sequence.

Useful examples of peptides that bind each other that may be employed in these and similar embodiments include SpyCatcher and SpyTag. The terms “SpyTag” and “SpyCatcher” (with “Spy” referring to the bacterium Streptococcus pyogenes) refer to a convenient protein-coupling tool that can join two polypeptides with greater efficacy than common protein-protein interactions. The SpyTag/SpyCatcher system may be used for binding, labeling or immobilizing proteins as it creates irreversible peptide ligations. SpyTag is a genetically encoded peptide that forms a spontaneous amide bond upon binding its genetically encoded partner SpyCatcher. SpyTag reacts with SpyCatcher under a wide range of conditions and the after reaction product is extremely stable. SpyCatcher and SpyTag are described in Zakeri et al., (Proc Natl Acad Sci USA. 2012; 109(12):E690-7) and PCT Pub. No. WO/2017/112784; the disclosures of which are incorporated herein by reference in their entirety.

Useful examples of protein coupling strategies that may be adapted to inducibly ligate, or otherwise associate, a degradation signal to a target signaling protein in a circuit of the present disclosure also include PROTACs. Proteolysis-targeting chimeras (PROTACs) are two-headed macromolecules include a first domain that binds a target protein and a second domain that is capable of engaging an E3 ubiquitin ligase. Methods of employing a PROTAC for targeted degradation which may be adapted for use in the herein described circuits and methods include but are not limited to e.g., those described in e.g., Raina & Crews, J Biol Chem. (2010) 285(15):11057-60; Raina & Crews, Curr Opin Chem Biol. (2017) 39:46-53; Coleman & Crews, Annual Review of Cancer Biology (2018) 2:41-58; Bondeson et al., Cell Chemical Biology (2018) 25(1):78-87; Neklesa et al., Pharmacology & Therapeutics. (2017) 174:138-144; the disclosures of which are herein incorporated by reference in their entirety. Ubiquibodies and peptide PROTACs are described in, e.g., Ludwicki et al, ACS Central Science 2019 5: 852-866; Portnoff et al, J. Biol. Chem. 2014 289: 7844-7855; Fan et al Nature Neuroscience 2014 17: 471 480; and Hines et al Proc. Natl. Acad. Sci. 2013 110: 8942-8947.

As will be readily understood, strategies and components for inducibly ligating a degradation signal to a target signaling protein in a circuit of the present disclosure may not be limited to those strategies and components specifically described herein and may, in some instances, adapt other protein coupling systems for use in the described circuits.

In some embodiments, an induced degradation strategy employed in a circuit of the present disclosure may include phosphorylation of a degron included in a target signaling protein. For example, a degron that may be phosphorylated by a kinase may be incorporated into a target signaling protein and a switch polypeptide may include the kinase. Accordingly, upon expression of the switch polypeptide, the kinase phosphorylates the degron triggering degradation of the target signaling protein. Correspondingly, when the switch polypeptide is not expressed, the kinase is not present, the degron is not phosphorylated, and the target signaling protein is not degraded.

Useful examples of phosphorylation-based induced degradation strategies that may be adapted for use in the circuits of the present disclosure include but are not limited to e.g., modular-phospho-degron strategies such as those described by Gordley et al. PNAS (2016) 113(47): 13528-13533; the disclosure of which is incorporated herein by reference in its entirety. For example, a modular phospho-degron may include an extended region of the Tecl transcription factor that is phosphorylated by the Fus3 mitogen-activated protein kinase (MAPK), inducibly binds the yeast Cdc4 E3 ubiquitin ligase complex, and contains an additional polylysine region that facilitates ubiquitinylation. The phospho-degron may include a mutationally optimized Cdc binding region that results in rapid degradation of an attached polypeptide upon MAPK activation. Accordingly, a target signaling protein may be configured to include the modular phospho-degron and a switch polypeptide may be configured to include the MAPK. Thus, upon expression of the switch polypeptide, the phospho-degron is phosphorylated by the MAPK and subsequently ubiquitinated, resulting in degradation of the target signaling protein. When the switch polypeptide is absent, the phospho-degron is not phosphorylated and the target signaling protein is not degraded.

As will be readily understood, strategies and components for phosphorylation-dependent inducible degradation of a target signaling protein in a circuit of the present disclosure may not be limited to those strategies and components specifically described herein and may, in some instances, adapt other phosphorylation-dependent systems for use in the described circuits.

In some embodiments, an induced degradation strategy employed in a circuit of the present disclosure may include an orthogonal proteasome system. For example, a switch polypeptide may be configured to include a proteasome that is heterologous to the cell type in which the switch polypeptide is expressed (i.e., a heterologous proteasome) and a targeted signaling protein may be configured to include a degradation tag specific for the heterologous proteasome. Accordingly, the proteasome and the degradation tag may constitute an orthogonal pair, such that, the tagged signaling protein is only degraded by the heterologous proteasome (i.e., the tag does not induce degradation of the targeted signaling protein by any host-derived (endogenous) protein degradation machinery).

Various orthogonal proteasome/tag pairs may be employed, e.g., depending on the cell that is modified to include the circuit. For example, in a eukaryotic cell an orthogonal proteasome/tag pair may include a prokaryotic proteasome and a prokaryotic degradation tag that signals degradation by the prokaryotic proteasome. In a prokaryotic cell an orthogonal proteasome/tag pair may include a eukaryotic proteasome and a eukaryotic degradation tag that signals degradation by the eukaryotic proteasome. In some instances, orthogonal proteasome/tag pairs may be synthetically derived, e.g., by mutation of the proteasome and/or the tag to render the pair orthogonal. In some instances, cross-species proteasome/tag pairs may be employed in an orthogonal proteasome system, such as e.g., a proteasome and degradation tag derived from a first species (e.g., a first bacterial species, a first eukaryote species, etc.) employed in a second species (e.g., a second bacterial species, a second eukaryote species, etc.).

Useful examples of orthogonal proteasome systems that may be adapted for use in a circuit of the present disclosure include but are not limited to e.g., those derived from bacteria (e.g., E. coli, M. florum, etc.), such as but not limited to e.g., ClpXP proteasome and ssrA tag systems, mfLon proteasome and pdt tag systems, and the like. Useful systems and components that may be adapted for use in the circuits of the present disclosure include but are not limited to e.g., those described in Cameron & Collins, Nat Biotechnol. 2014; 32(12): 1276-1281 and Grilly et al. Molecular Systems Biology. 2007; 3:127; the disclosures of which are incorporated herein by reference in their entirety. Accordingly, in some instances, a switch polypeptide may include a proteasome of an orthogonal proteasome/tag pair and a targeted signaling protein may include the degradation tag of the orthogonal proteasome/tag pair. A circuit employing an orthogonal proteasome/tag pair may be introduced into a cell where the proteasome and/or the tag are heterologous (i.e., not endogenous) to the cell.

As will be readily understood, strategies and components for orthogonal proteasome-based inducible degradation of a target signaling protein in a circuit of the present disclosure may not be limited to those strategies and components specifically described herein and may, in some instances, adapt other orthogonal proteasome systems for use in the described circuits.

In some embodiments, an induced degradation strategy employed in a circuit of the present disclosure may include a system of induced localization to the proteasome. For example, in some embodiments, a switch polypeptide may include a domain that, when expressed, localizes, or can be induced to localize, a signaling protein to the proteasome, thereby inducing degradation of the signaling protein. Induced localization of the signaling protein to the proteasome may, in some instances, be ubiquitin independent. Put another way, induced localization of the signaling protein to the proteasome may bypass the ubiquitination step.

As an example, a switch polypeptide is configured to include a first member of a dimerization pair fused to a proteasome and a target signaling protein is modified to include a second member of the dimerization pair. First and second members of a dimerization pair may directly bind each other (i.e., may directly dimerize) or may dimerize via a dimerization mediator (i.e., a dimerizer). Any two convenient polypeptide domains that are capable of forming a complex with each other may be selected for use as first and second dimerization domains. For example, where the first and second members of the dimerization pair directly bind each other, the expression of the switch polypeptide that includes the first member of the dimerization pair fused to the proteasome induces localization of the target signaling protein to the proteasome by direct binding between the first and second members of the dimerization pair, thereby resulting in degradation of the signaling protein. When the switch polypeptide is not expressed, the target signaling protein is not localized to the proteasome.

Where the first and second members of the dimerization pair are dimerized by a dimerization mediator, the targeted signaling protein (that includes the second member of the dimerization pair) may be localized to the proteasome in the presence of both the switch polypeptide that includes the first member of the dimerization pair fused to the proteasome and the dimerization mediator. Accordingly, where a chemical inducer of dimerization (CID) is employed to induce dimerization of the proteasome-fused switch polypeptide and the targeted signaling protein, the presence or absence of the CID may control whether the circuit is capable or incapable, respectively, of generating feedback. Where dimerization domains that directly bind each other are employed, the generation of feedback may be independent of the presence of any dimerization-inducing molecule.

As a non-limiting example of the induced localization to the proteasome strategy of induced degradation, Fprl (a peptide that has been shown to bind high affinity with lipophilic macrolide rapamycin) is fused (e.g., C-terminally fused) to a proteasome subunit and the ligand-binding domain of Tor1 (which binds Fpr1-bound rapamycin) is fused to a target signaling protein. Upon expression of the Fpr1-tagged proteasome subunit in the presence of rapamycin, the Tor1-tagged signaling protein is localized to the proteasome, resulting in localization-induced degradation of the signaling protein. In some instances, Fpr1 and Tor1 may be substituted for dimerization domains that directly bind each other, thereby negating the necessity for the dimerization mediator (e.g., rapamycin). In some instances, dimerization domains of a direct dimerizing pair may be substituted, negating the need for dimerization mediator. Examples of induced proteasome localization strategies, as well as Fpr1 and Tor1 domains, that may be adapted for use in the circuits of the present disclosure include but are not limited to e.g., those described in Janse et al. J Biol Chem. 2004; 279(20):21415-20; the disclosure of which is incorporated herein by reference in its entirety.

Non-limiting examples of useful dimerization domains include, but are not limited to, protein domains of the iDimerize inducible homodimer (e.g., DmrB) and heterodimer systems (e.g., DmrA and DmrC) and the iDimerize reverse dimerization system (e.g., DmrD) (Takara Bio Inc.) (See also Clackson et al. (1998) Redesigning an FKBP-ligand interface to generate chemical dimerizers with novel specificity. Proc. Natl. Acad. Sci. USA 95(18): 10437-10442; Crabtree, G. R. & Schreiber, S. L. (1996) Three-part inventions: intracellular signaling and induced proximity. Trends Biochem. Sci. 21(11): 418-422; Jin et al. (2000) In vivo selection using a cell-growth switch. Nat. Genet. 26(1): 64-66; Castellano et al. (1999) Inducible recruitment of Cdc42 or WASP to a cell-surface receptor triggers actin polymerization and filopodium formation. Curr. Biol. 9(7): 351-360; Crabtree et al. (1997) Proximity and orientation underlie signaling by the non-receptor tyrosine kinase ZAP70. Embo. J. 16(18): 5618-5628; Muthuswamy et al. (1999) Controlled dimerization of ErbB receptors provides evidence for differential signaling by homo- and heterodimers. Mol. Cell. Biol. 19(10): 6845-6857). As an ordinarily skilled artisan will readily understand, various other dimerization domains may be employed.

As will be readily understood, strategies and components for induced proteasome-localization-based inducible degradation of a target signaling protein in a circuit of the present disclosure may not be limited to those strategies and components specifically described herein and may, in some instances, adapt other induced proteasome localization systems for use in the described circuits.

Various degrons may be employed in the inducible degradation strategies described herein. Degrons include portions of proteins that signal and/or target for degradation (or otherwise increase the degradation rate of) the protein to which the degron is attached or otherwise associated (e.g., grafted onto). Non-limiting examples of degrons include short amino acid sequences, structural motifs, exposed amino acids, and the like. Degrons may be prokaryote or eukaryote derived and may be employed in naturally occurring or non-naturally occurring (i.e., recombinant) forms. Degrons may be post-translationally modified to target a protein for degradation where such post-translational modifications include but are not limited to e.g., ubiquitination, proteolytic cleavage, phosphorylation, methylation, ADP-ribosylation, ampylation, lipidation, alkylation, nitrosylation, succinylation, sumoylation, neddylation, isgylation, etc.

Useful degrons include ubiquitin-dependent degrons and ubiquitin-independent degrons.

For example, in some instances, a protein may be targeted for ubiquitin-independent proteasomal degradation by attachment of an ornithine decarboxylase (ODC) degron, including but not limited to e.g., a mammalian ODC such as e.g., a rodent ODC, including but not limited to e.g., the c-terminal mouse ODC (cODC). In some instances, useful degrons include those described in Takeuchi et al., Biochem. J (2008) 410:401-407 and/or Matsuzawa et al., PNAS (2005) 102(42):14982-7; the disclosures of which are incorporated herein by reference in their entirety. In some instances, a protein may be targeted for ubiquitin-independent proteasomal degradation by post-translational modification (including but not limited to e.g., proteolytic cleavage, phosphorylation, methylation, ADP-ribosylation, ampylation, lipidation, alkylation, nitrosylation, succinylation, sumoylation, neddylation, isgylation, etc.) of a degron, where such modification leads, directly or indirectly, to partial or complete unfolding of the protein or other mechanisms that lead to degradation of the protein.

In some instances, a degron employed in the herein described circuits may include a ubiquitin-independent degradation signal, where such signals may vary. For example, in some instances, a ubiquitin-independent degradation signal may include a dipeptide motif, such as e.g., a cysteine-alanine (i.e., CA) dipeptide motif. In some instances, a ubiquitin-independent degradation signal may include only a dipeptide motif. In some instances, a ubiquitin-independent degradation signal may include amino acid residues in addition to a dipeptide motif, such as but not limited to e.g., a LXMSCAQE motif, where X may be any amino acid or a LXMSCAQES motif, where X may be any amino acid. In some instances, a LXMSCAQE motif or a LXMSCAQES motif may include where X is any amino acid except proline.

Accordingly, in some instances, a degradation signal of a degron may include a sequence selected from: LPMSCAQES where the final S is present or absent, LAMSCAQES where the final S is present or absent, LVMSCAQES where the final S is present or absent, LSMSCAQES where the final S is present or absent, LEMSCAQES where the final S is present or absent, and LKMSCAQES where the final S is present or absent. In some instances, a degradation signal of a degron may include a MSCAQE sequence or a MSCAQES sequence.

Ubiquitin-dependent degrons include, but are not limited to, e.g., PEST (proline (P), glutamic acid (E), serine (S), and threonine (T)) sequence-containing degrons, as well as those degrons described in Melvin et al. (PLoS One. (2013) 29; 8(10):e78082); the disclosure of which is incorporated herein by reference in its entirety, including degrons identified as Bonger and those described as derived from TAZ, HIF-1α, iNOS, SRC3, Cyclin D1, IFNAR1, p53, and β-Catenin.

Useful degrons may also include E3 ubiquitin ligase domains. Such degrons are often defined as the substrate site that is recognized by E3 ubiquitin ligases and a variety of such degrons, including short peptide motifs and specific structural elements, have been characterized. Non-limiting examples of E3 ligase/degrons and the corresponding motif patterns include: APC/C (DBOX), primary motif .R..L..[LIVM].; APC/C (KEN), primary motif .KEN.; APC/C (ABBA), primary motif [FIVL].[ILMVP][FHY].[DE].{0,3}[DEST]; APCC_TPR_1, primary motif .[ILM]R$; CBL (PTK), primary motif [DN].Y[ST]..P; CBL (MET), primary motif DYR; COP1, primary motif [DE][DE].{2,3}VP[DE]; CRL4_CDT2_1, primary motif [NQ]{0,1}..[ILMV][ST][DEN][FY][FY].{2,3}[KR]{2,3}[\hat{ }DE]; CRL4_CDT2_2, primary motif [NQ]{0,1}..[ILMV]T[DEN][HMFY][FMY].{2,3}[KR]{2,3}[\hat{ }DE]; Kelch_KEAP1_1, primary motif [DNS].[DES][TNS]GE; Kelch_KEAP1_2, primary motif QD.DLGV; Kelch_actinfilin, primary motif [AP]P[MV][IM]V; Kelch_KLHL3, primary motif E.EE.E[AV]DQH; MDM2_SWIB, primary motif F[\hat{ }P]{3}W[\hat{ }P]{2,3}[VIL]; Nend_Nbox_1, primary motif \hat{ }M{0,1}[FYLIW][\hat{ }P]; Nend_UBRbox_1, primary motif \hat{ }M{0,1}[RK][\hat{ }P].; Nend_UBRbox_2, primary motif \hat{ }M{0,1}([ED]).; Nend_UBRbox_3, primary motif \hat{ }M{0,1}([NQ]).; Nend_UBRbox_4, primary motif \hat{ }M{0,1}(C).; ODPH_VHL_1, primary motif [IL]A(P).{6,8}[FLIVM].[FLIVM]; SCF_COI1_1, primary motif ..[RK][RK].SL..F[FLM].[RK]R[HRK].[RK].; SCF_FBW7_1, primary motif [LIVMP].{0,2}(T)P..([ST]); SCF_FBW7_2, primary motif [LIVMP].{0,2}(T)P..E; SCF_SKP2-CKS1_1, primary motif ..[DE].(T)P.K; SCF_TIR1_1, primary motif .[VLIA][VLI]GWPP[VLI]...R.; SCF-TRCP1, primary motif D(S)G.{2,3}([ST]); SIAH, primary motif .P.A.V.P[\hat{ }P]; SPOP, primary motif [AVP].[ST][ST][ST]; where ‘.’ specifies any amino acid type, ‘[X]’ specifies the allowed amino acid type(s) at that position, ‘\hat{ }X’ at the beginning of the pattern specifies that the sequence starts with amino acid type X, ‘[\hat{ }X]’ means that the position can have any amino acid other than type X, numbers specified as the following ‘X{x,y}’, where x and y specify the minimum and maximum number of ‘X’ amino acid type required at that position. ‘$’ sign implies the C-terminal of the protein chain. Degrons that include E3 ubiquitin ligase domains are described in Guharoy et al., Nature Communications (2016) 7:10239; the disclosure of which is incorporated herein by reference in its entirety. In some instances, useful degrons may include those degrons that contain signals for ER-associated degradation (ERAD), including but not limited to e.g., those described in Maurer et al., Genes Genomes & Genetics (2016) 6:1854-1866; the disclosure of which is incorporated herein by reference in its entirety. In some instances, useful degrons may also include drug-inducible degrons, such as but not limited to e.g., the auxin inducible degron (AID) which utilizes a specific E3 ubiquitin ligase (e.g., as described in Nishimura et al., Nature Methods (2009) 6(12):917-922; the disclosure of which is incorporated herein by reference in its entirety).

As will be readily understood, degrons that include E3 ubiquitin ligase domains will vary and circuit of the present disclosure may not be limited to use of those E3 ubiquitin degrons specifically described herein.

In some instances, inducible degradation in the circuits of the present disclosure may employ direct caging of ubiquitin that, when uncaged, localizes the polypeptide containing the uncaged ubiquitin to the proteasome. For example, degrons can be tuned by modifying the relative spacing between the components of the degron (see e.g., Inobe et al., Nature Chemical Biology, 7(3), 161-167; the disclosure of which is incorporated herein by reference in its entirety). Thus, through such modification, a degron may be rendered non-functional or at least minimally function. Degradation function of the modified degron can then be restored by directly localizing or ligating (e.g., via dimerization) ubiquitin to the latent deactivation domain-containing signaling protein. Any convenient method of ligating/localizing a ubiquitin protein to a modified degron may be employed in such embodiments. For example, in some instances, a dimerizer pair may be employed with one member of the pair present in the latent deactivation domain and the other member attached to a ubiquitin protein. In some instances, a leucine zipper pair may be employed with one member of the pair present in the latent deactivation domain and the other member attached to a ubiquitin protein. In some instances, a SpyCatcher/SpyTag pair may be employed with one member of the pair present in the latent deactivation domain and the other member attached to a ubiquitin protein. Such examples are for illustrative purposes only and are not intended to be limiting.

Other useful examples of degrons that may be employed in inducible degradation strategies adapted for use in the circuits of the present disclosure include but are not limited to e.g., N-end degrons (such as but not limited to e.g., those described in Tasaki & Kwon, Trends in Biochemical Sciences (2007) 32(11):520-528, the disclosure of which is incorporated herein by reference in its entirety); unstructured regions (such as but not limited to e.g., those described in Chung et al., Nat Chem Biol. 2015; 11(9): 713-720, the disclosure of which is incorporated herein by reference in its entirety); ligand induced degradation (LID) and destabilization domain (DD) domains (such as but not limited to e.g., those described in Bonger et al., Nat Chem Biol. 2012; 7(8): 531-537; Grimley et al., Bioorg. Med. Chem. Lett. (2008) 18: 759-761; and Chu et al. Bioorg. Med. Chem. Lett. (2008) 18: 5941-5944; Iwamoto et al., Chemistry & Biology (2010) 17: 981-988; the disclosures of which are incorporated herein by reference in their entirety); prokaryotic proteasome recognition sequences such as, e.g., ssrA and mf-Lon (such as those described in Cameron et al., (2014) Nature biotechnology 32(12): 1276-1281, the disclosure of which is incorporated herein by reference in its entirety); and the like.

An example of an inducible degradation system adapted for use in a circuit demonstrating feedback control of the subject circuit is the degronLOCKR system that includes a caged degron that is uncaged by an expressed key polypeptide. The degronLOCKR system, and circuits employing degronLOCKR, are described in co-pending provisional applications Ser. Nos. 62/789,418 (filed on Jan. 7, 2019), 62/850,336 (filed on May 20, 2019) and 62/789,351, filed on Jan. 7, 2019., as well as U.S. Provisional Patent Application Nos.: 62/700,681 (filed Jul. 19, 2018) 62/785,537 (filed Dec. 27, 2018) and 62/788,398 (filed Jan. 4, 2019); the disclosures of which are incorporated herein by reference in their entirety. In some instances, circuits and/or methods of the present disclosure exclude the use of caged degron systems and/or components of caged degron systems and/or the DegronLOCKR system and/or components of the DegronLOCKR system. Accordingly, in some instances, a latent deactivation domain of the present disclosure is not a caged degron. In some instances, a latent deactivation domain of the present disclosure is not a DegronLOCKR. In some instances, a latent deactivation domain of the present disclosure does not comprise a LOCKR domain. In some instances, a latent deactivation domain of the present disclosure does not comprise a degronLOCKR polypeptide.

As summarized above, in some instances useful strategies for deactivation may include inducible localization. Thus, a latent deactivation domain of a circuit of the present disclosure may include a domain that inducibly localizes a signaling protein to a location that renders the signaling protein inactive. Put another way, in the presence of a switch polypeptide, a latent deactivation domain may become active such that the deactivation domain localizes an attached signaling protein to a portion of the cell where the signaling protein is inactive. Various different strategies for inducible localization-based deactivation may be employed in the herein described circuits.

For example, in some embodiments, a switch polypeptide may be configured to include a first member of a binding pair linked to a sequestration domain and a targeted signaling protein may include a second member of the binding pair. Accordingly, upon expression of the switch polypeptide and binding of the first and second members of the binding pairs, the targeted signaling protein may be sequestered and deactivated. In the absence of the switch polypeptide, the targeted signaling protein is not sequestered and thus remains active (i.e., is not deactivated).

As used herein, the term “sequestration domain” will generally refer to any protein domain that when attached to a polypeptide and available (i.e., not caged or otherwise inaccessible) results in the localization of the polypeptide to a location in a cell where the polypeptide is not capable of performing its primary function. For example, in a case where the polypeptide is transcription factor and its primary function is to drive expression of one or more target genes, a sequestration domain may function to localize the polypeptide to a location of the cell away from the nucleus to prevent the polypeptide from performing its function of driving expression of one or more target genes. However, the polypeptide having a transcription factor function may be configured such that, in the absence of the switch polypeptide, the polypeptide is capable of localizing to the nucleus of the cell to drive expression of one or more of its target genes.

The strategy employed for inducible localization may depend on the mechanism of action of the polypeptide targeted and useful strategies will not be limited to sequestering of a transcription factor away from the nucleus. Accordingly, various sequestration domains may be employed in the inducible localization-based deactivation strategies described herein. For example, in some instances, a sequestration domain employed may localize an attached polypeptide to various locations of the cell including but not limited to e.g., the plasma membrane, the mitochondria, the peroxisome, the vacuole, the actin cytoskeleton, and the like. Accordingly, sequestration domains may include a tag that when present and accessible induces the localization of the attached polypeptide to a specific location within the cell. Useful examples of such tags may include but are not limited to e.g., a plasma membrane-targeting tag, a mitochondrial membrane-targeting tag, a peroxisome-targeting tag, a vacuole-targeting tag, an actin-cytoskeleton-targeting tag, and the like.

In some embodiments of the herein described circuits, a targeted signaling protein may include a first member of a binding pair and a switch polypeptide may include a second member of the binding pair linked to a sequestration domain, including but not limited to e.g., where the sequestration domain includes a plasma membrane-targeting tag. a mitochondrial membrane-targeting tag, a peroxisome-targeting tag, a vacuole-targeting tag, an actin-cytoskeleton-targeting tag, or the like. In some instances, other useful targeting tags may include but are not limited to e.g., nuclear localization tags, nuclear export tags, and the like.

In some embodiments, a useful inducible localization strategy employed in a circuit of the present disclosure may include a switch polypeptide that includes first leucine zipper domain fused to a localization tag and a second leucine zipper fused to a targeted signaling protein of a signaling pathway. For example, the targeted signaling protein may be, but is not limited to, a transcription factor member of a signaling pathway and the localization tag may localize to a cellular location other than the nucleus, such as but not limited to e.g., the plasma membrane. Thus, deactivation of the transcription factor target signaling protein may be controlled by the presence of the switch polypeptide. In the presence of the switch polypeptide the first leucine zipper binds the second leucine zipper and the transcription factor target signaling protein is sequestered away from (e.g., outside of) the nucleus thereby deactivating the signaling protein. In the absence of the switch polypeptide the signaling protein is not sequestered and deactivation is not induced. Thus, the signaling protein may perform its signaling function. This strategy of induced sequestration may be referred to, in some instances, elsewhere herein as “anchor away”.

In some instances, useful leucine zipper binding pairs and sequestration domains that may be employed in anchor away strategies employed in the circuits of the present disclosure may include but are not limited to e.g., those described in Chen et al. ACS Synth. Biol., 2015, 4(11):1205-1216, the disclosure of which is incorporated herein by reference in its entirety. Examples of potential heterodimerization domains are described in the following publications. These domains can be used for recruiting a degron, localizing proteins (anchor away), or as dominant negatives. See, e.g., Thompson et al, ACS Synthetic Biology 2012 1: 118-129 and Chen et al Nature 2019 565: 106-111.

Useful binding pairs that may be employed in inducible localization strategies are not limited to leucine zipper domains and useful domains thereof may vary. First and second members of a binding pair may directly bind each other (i.e., may directly bind) or may bind via a binding mediator. Any two convenient polypeptide domains that are capable of forming a complex with each other may be selected for use as first and second members of a binding pair. In some instances, polypeptide dimerization domains, including but not limited to those described elsewhere herein, may be employed as first and second members of a binding pair, including where the first and second members of the dimerization pair directly bind each other (i.e., may directly dimerize) or dimerize via a dimerization mediator (i.e., a dimerizer).

Useful examples of binding pairs, and the first and second members thereof, may also include but are not limited to e.g., the specific binding pairs and dimerization pairs described in PCT Pub. Nos. WO2014/127261 and WO2017/120546, the disclosures of which are incorporated herein by reference in their entirety.

In some instances, a strategy for inducible localization employed in a circuit of the present disclosure may include a caged sequestration domain. As used herein, by “caged sequestration domain” is generally meant a multi-domain polypeptide that includes a localization tag and a cage domain that prevents the localization tag from triggering localization of the polypeptide, and any attached protein, according to the normal function of the localization tag. For example, a polypeptide that includes a caged sequestration domain that includes a plasma membrane-targeting tag may not localize to the plasma membrane when the tag remains caged but may localize the polypeptide, and any attached protein, to the plasma membrane when the tag is uncaged.

In another example, a polypeptide that includes a caged sequestration domain that includes a nuclear localization tag (such as but not limited to e.g., a nuclear localization sequence(NLS)) may not localize to the nucleus when the tag remains caged but may localize the polypeptide, and any attached protein, to the nucleus when the tag is uncaged. In some instances, this strategy for induced localization may find use where the signaling protein targeted functions in the signaling pathway outside of the nucleus, e.g., in the cytoplasm and/or plasma membrane, of the cell. In such embodiments, any convenient nuclear localization sequence may be employed.

In another example, a polypeptide that includes a caged sequestration domain that includes a nuclear export tag (such as but not limited to e.g., a nuclear export sequence (NES)) may localize to the nucleus when the tag remains caged but may localize the polypeptide, and any attached protein, to outside nucleus when the tag is uncaged. In some instances, this strategy for induced localization may find use where the signaling protein targeted functions in the nucleus of the cell. In such embodiments, any convenient nuclear export sequence may be employed. In some instances, a caged sequestration domain that includes a nuclear export tag may exclude a LOCKR domain, including but not limited to e.g., where the caged sequestration domain does not comprise a nesLOCKR polypeptide.

Any useful strategy for caging/uncaging of a localization tag may be employed in the relevant embodiments. Including but not limited to e.g., protection of the localization tag by a domain that includes a proteolytic cleavage site that is cleavable by a protease included in a switch polypeptide. For example, a localization tag may be attached to a signaling protein and the localization tag may be caged by a protection domain that includes a proteolytic cleavage site that is cleavable by a protease included in a switch polypeptide. In the absence of switch polypeptide, the protease is not present and the protection domain prevents the localization tag from triggering localization of the signaling protein. In the presence of the switch polypeptide, the protease cleaves the proteolytic cleavage site, uncaging, or otherwise deprotecting, the localization tag and triggering localization of the signaling protein according to the localization tag.

In some embodiments, a LOCKR-based cage may be employed in an inducible localization strategy of a circuit of the present disclosure. For example, in some instances, a signaling protein may be configured to include a localization tag (such as but not limited to e.g., an NLS or NES) that may be caged by LOCKR and uncaged by a switch polypeptide that includes a key polypeptide. In some embodiments, a LOCKR-based cage may not be employed in an inducible localization strategy of a circuit of the present disclosure. For example, in some instances, a signaling protein may be configured to include a localization tag (such as but not limited to e.g., an NLS or NES) that is not caged by LOCKR domain and the latent deactivation domain-containing protein may not comprise a LOCKER domain, e.g., a nesLOCKR or nlsLOCKER. LOCKR domains, and circuits employing the LOCKR system, are described in co-pending provisional applications identified by attorney docket numbers UCSF-578PRV and MBHB 18-1783-PRO, filed Jan. 7, 2019, as well as U.S. Provisional Patent Application Nos.: 62/700,681 (filed Jul. 19, 2018), 62/785,537 (filed Dec. 27, 2018), and 62/788,398 (filed Jan. 4, 2019); the disclosures of which are incorporated herein by reference in their entirety.

As summarized above, useful strategies for induced deactivation of a signaling protein of a circuit of the present disclosure may include protein splitting. As used herein, by “protein splitting” is generally meant splitting a polypeptide to render the polypeptide non-functional or negate at least one function of the polypeptide. In some instances, a polypeptide may be reversibly split such that the split portions of the polypeptide may be rejoined to render the polypeptide functional or to restore at least one function of the polypeptide that was abolished by the split. In some instances, a polypeptide may be irreversibly split such that the split portions of the polypeptide may not be rejoined to render the polypeptide functional or to restore a function of the polypeptide that was abolished by the split.

In some embodiments, a polypeptide of a signaling protein is split (abolishing at least one function of the signaling protein) and each half of the split polypeptide is fused to a member of a binding pair such that the split signaling protein may be reconstituted upon binding of the binding pair members. For example, a signaling protein may be split into a first half and a second half and the first half may be fused to a first member of a binding pair and the second half of the split signaling protein may be used to a second member of the binding pair. Upon binding of the first and second members of the binding pair to each other, the signaling protein is reconstituted restoring the at least one function of the signaling protein that was abolished.

In a circuit of the present disclosure, a switch polypeptide may be configured to include binding pair member that outcompetes one or both of the binding pair members used to reconstitute the split polypeptide. Accordingly, in the presence of the switch polypeptide, the binding of the first and second members of the binding pair may be interrupted by the switch polypeptide, thereby preventing reconstitution of the signaling protein and resulting in deactivation of the signaling protein.

In such instances, the binding domain of the switch polypeptide may be referred to herein as a “dominant negative” domain. A dominant negative domain may competitively outcompete the binding between domains of a latent deactivation domain attached to, or otherwise incorporated into, a signaling protein of a circuit of the present disclosure. Accordingly, in some instances, a latent deactivation domain may be said to include a competitive binding domain that binds non-covalently with a domain of a signaling protein. Such a “competitive binding domain” may be displaced in the presence of the binding domain of a switch polypeptide, thus resulting in deactivation of the signaling protein. In the absence of the switch polypeptide, the competitive binding domain binds its binding partner in the split signaling protein, reconstituting the signaling protein and rendering it capable of performing its function in the signaling pathway.

In some embodiments, a signaling protein, such as a transcription factor or a kinase, may be split such that when the split portions of the protein are not associated with one another the signaling protein does not perform its function, i.e., it does not induce transcription of a target, it does not catalyze phosphorylation of a target, etc. A first half of the split signaling protein may be fused to a first leucine zipper and a second half of the split signaling protein may be fused to a second leucine zipper such that the singling protein can be reconstituted upon binding of the first leucine zipper to the second leucine zipper. A dominant negative leucine zipper, i.e., a third leucine zipper that binds to either the first or second leucine zipper with a higher affinity than the interaction between the first and second, is incorporated into the switch polypeptide. Thus, the switch polypeptide may outcompete one half of the split signaling protein rendering it non-functional, e.g., incapable of performing a transcription factor function, incapable of performing a kinase function, or the like. Useful examples of leucine zippers and the use of a dominant negative leucine zipper to outcompete the binding of two lower affinity leucine zippers include but are not limited to e.g., the heterologous mammalian bZIP (CEBPa) transactivator and high affinity 3HF dominant-negative inhibitor described in Buchler & Cross. Molecular Systems Biology (2009) 5:272; the disclosure of which is incorporated herein by reference in its entirety.

In some embodiments, useful protein splitting strategies include cleavage of protease cleavage sites placed between dimerization domains that dimerize portions of a split signaling protein to reconstitute the signaling protein. For example, a signaling protein may be split into a first portion having a first member of a dimerization pair and a second portion that includes a second member of the dimerization pair. Either or both of the members of the dimerization pair may be configured to contain a protease cleavage site that is cleaved by a protease. Accordingly, upon expression of a switch polypeptide that includes the protease, the protease cleavage site(s) is/are cleaved, splitting the reconstituted signaling protein thereby rendering it inactive.

In some instances, the dimerization pair includes leucine zippers, such as antiparallel leucine zippers, that dimerize the split signaling protein. Thus, the signaling protein is split into a first portion having a first leucine zipper of an antiparallel leucine zipper pair and a second portion that includes the second leucine zipper of the pair. Either or both of the leucine zippers of the pair may be configured to contain a protease cleavage site that is cleaved by a protease. Accordingly, upon expression of a switch polypeptide that includes the protease, the protease cleavage site(s) is/are cleaved, splitting the reconstituted signaling protein thereby rendering it inactive.

Useful examples of protease strategies that may be employed include but are not limited to e.g., where the switch polypeptide may include a TEV protease that will cleave a protease cleavage site present in a dimerization domain of a split signaling protein. Cleavage by the TEV protease destroys the ability of the dimerization domains to reconstitute the split signaling protein, thereby deactivating and re-splitting the reconstituted split signaling protein. Accordingly, in the absence of the TEV protease-containing switch polypeptide the protease cleave site(s) remain(s) uncleaved and the split signaling protein remains reconstituted and active. In some instances, components and configurations that may be employed to proteolytically cleave a signaling protein to result in a deactivated split signaling protein may include those employed in the CHOMP (circuits of hacked orthogonal modular proteases) system as described in Gao et al., Science. 2018; 361(6408):1252-1258; the disclosure of which is incorporated herein by reference in its entirety. As will be readily understood, in some instances, other encoded proteases, corresponding protease cleavage sites, and/or dimerization domains may be substituted for those explicitly described.

As will also be readily understood, various configurations of induced deactivation of a signaling protein employing protein splitting may be used in a circuit of the present disclosure.

As summarized above, circuits of the present disclosure may include a switch polypeptide, the expression of which may be controlled by a regulatory sequence to which a sequence encoding the switch polypeptide is operably linked. The term “switch polypeptide”, as used herein, generally refers to a polypeptide that, when expressed in the presence of a corresponding latent deactivation domain, activates the latent activation domain. Activation of the latent deactivation domain thereby triggers deactivation of the polypeptide to which the deactivation domain is linked or otherwise incorporated and any other attached proteins, such as e.g., an attached signaling protein. The configuration of switch polypeptides will vary, e.g., depending on the latent component which the switch is designed to activate.

Switch polypeptides configured to function with a particular latent deactivation domain may, in some instances, be configured as an orthogonal system. By “orthogonal system” is generally meant that a particular switch polypeptide functions together with a particular latent deactivation domain, but the switch polypeptide does not necessarily function with other latent deactivation domains and/or the latent deactivation domain does not necessarily function with other switch polypeptides. Accordingly, two or more different orthogonal systems of switch polypeptide and latent deactivation domain may function together, e.g., simultaneously, in the same organism or cell without interfering. Put another way, a first switch polypeptide of a first orthogonal system may function to activate a first latent deactivation domain of the first system, while the switch polypeptide does not substantially interfere with the function of any component of a second orthogonal system (e.g., second switch polypeptide, second latent deactivation domain, etc.). Orthogonal systems may be employed, in some instances, to allow for the parallel operation of two or more molecular feedback circuits, including e.g., two or more molecular feedback circuits that each modulate a different signaling pathway, two or more molecular feedback circuits that each modulate a different component of the same signaling pathway, and the like.

Each switch polypeptide and latent deactivation domain need not necessarily be configured into orthogonal pairs. For example, in some instances, two or more different switch polypeptides may function to activate the same latent deactivation domain. Correspondingly, in some instances, two or more different latent deactivation domains may be configured to be activated by the same switch polypeptide.

Linkers

Polypeptides employed in the circuits of the present disclosure may or may not include peptide linkers. For example, in some instances, two domains of a subject polypeptide may be joined by a peptide linker. Correspondingly, nucleic acid sequences encoding components of the circuits of the present disclosure may be joined by sequence encoding a peptide linker.

A peptide linker can vary in length of from about 3 amino acids (aa) or less to about 200 aa or more, including but not limited to e.g., from 3 aa to 10 aa, from 5 aa to 15 aa, from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A peptide linker can have a length of from 3 aa to 30 aa, e.g., 3, 4, 5, 6, 7, 8, 9, 10, 11, 12, 13, 14, 15, 16, 17, 18, 19, 20, 21, 22, 23, 24, 25, 26, 27, 28, 29, or 30 aa. A peptide linker can have a length of from 5 aa to 50 aa, e.g., from 5 aa to 40 aa, from 5 aa to 35 aa, from 5 aa to 30 aa, from 5 aa to 25 aa, from 5 aa to 20 aa, from 5 aa to 15 aa or from 5 aa to 10 aa.

Suitable linkers can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary linkers include glycine polymers (G)n, glycine-serine polymers (including, for example, (GS)n, (GSGGS)n (SEQ ID NO: 1) and (GGGS)n (SEQ ID NO: 2), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO: 3), GGSGG (SEQ ID NO: 4), GSGSG (SEQ ID NO: 5), GSGGG (SEQ ID NO: 6), GGGSG (SEQ ID NO: 7), GSSSG (SEQ ID NO: 8), and the like.

Signaling Pathways

As summarized above, various signaling pathways, including native and synthetic signaling pathways may be modulated using the herein described molecular circuits. Suitable signaling pathways include those that are modulated (e.g., activated, repressed, etc.) by one or more inputs to produce one or more outputs. Inputs and outputs of signaling pathways may vary and may include endogenous (e.g., native) inputs or outputs of signaling pathways and heterologous (e.g., engineered or synthetic) signaling pathway inputs and outputs.

In some instances, an input of a signaling pathway relevant to a circuit of the present disclosure may include an intracellular signal, including e.g., where the output of the pathway may be intracellular or intercellular. In some instances, an output of a signaling pathway relevant to a circuit of the present disclosure may include an intracellular signal, including e.g., where the input of the pathway may be intracellular or intercellular. In some instances, an input of a signaling pathway relevant to a circuit of the present disclosure may include an intercellular signal, including e.g., where the output of the pathway may be intracellular or intercellular. In some instances, an output of a signaling pathway relevant to a circuit of the present disclosure may include an intercellular signal, including e.g., where the input of the pathway may be intracellular or intercellular.

In some instances, both the input and the output of a signaling pathway relevant to a circuit of the present disclosure may include intracellular signals. In some instances, both the input and the output of a signaling pathway relevant to a circuit of the present disclosure may include intercellular signals.

Suitable non-limiting examples of native signaling pathways that may be modulated using a circuit of the present disclosure include but are not limited to e.g., the AKT signaling pathway, the Akt/PKB signaling pathway, the AMPK signaling pathway, the apoptosis signaling pathway, the BMP signaling pathway, the cAMP-dependent pathway, the estrogen signaling pathway, the hedgehog signaling pathway, the hippo signaling pathway, an immune activation pathway, an immune suppression pathway, an immune cell differentiation pathway, an insulin signal transduction pathway, the JAK-STAT signaling pathway, the MAPKIERK signaling pathway, the mTOR signaling pathway, the NF-κB signaling pathway, the nodal signaling pathway, the notch signaling pathway, the p53 signaling pathway, the PI3K signaling pathway, the TGF beta signaling pathway, the TLR signaling pathway, the TNF signaling pathway, the VEGF signaling pathway, the Wnt signaling pathway, and the like.

Suitable non-limiting examples of pathways, the components of which may be modified to include a latent deactivation domain as described herein, also include those PANTHER (Protein ANalysis THrough Evolutionary Relationships) pathways described as part of the Gene Ontology Phylogenetic Annotation Project, descriptions of which (including descriptions of the components of such pathways) are available online at www(dot)pantherdb(dot)org. Non-limiting examples include 2-arachidonoylglycerol biosynthesis, the 5HT1 type receptor mediated signaling pathway, the 5HT2 type receptor mediated signaling pathway, the 5HT3 type receptor mediated signaling pathway, the 5HT4 type receptor mediated signaling pathway, 5-Hydroxytryptamine biosynthesis, 5-Hydroxytryptamine degredation, Acetate utilization, the Activin beta signaling pathway, the Adenine and hypoxanthine salvage pathway, Adrenaline and noradrenaline biosynthesis, Alanine biosynthesis, Allantoin degradation, the ALP23B signaling pathway, the Alpha adrenergic receptor signaling pathway, the Alzheimer disease-amyloid secretase pathway, the Alzheimer disease-presenilin pathway, Aminobutyrate degradation, Anandamide biosynthesis, Anandamide degradation, Androgen/estrogene/progesterone biosynthesis, the Angiogenesis pathway, Angiotensin II-stimulated signaling through G proteins and beta-arrestin, the Apoptosis signaling pathway, Arginine biosynthesis, Ascorbate degradation, Asparagine and aspartate biosynthesis, ATP synthesis, Axon guidance mediated by netrin, Axon guidance mediated by semaphorins, Axon guidance mediated by Slit/Robo, the B cell activation pathway, the Betal adrenergic receptor signaling pathway, the Beta2 adrenergic receptor signaling pathway, the Beta3 adrenergic receptor signaling pathway, Biotin biosynthesis, Blood coagulation, the BMP/activin signaling pathway, Bupropion degradation, the Cadherin signaling pathway, Coenzyme A linked carnitine metabolism, Carnitine metabolism, CCKR signaling, the Cell cycle, Cholesterol biosynthesis, Chorismate biosynthesis, Circadian clock system, Cobalamin biosynthesis, Coenzyme A biosynthesis, the Cortocotropin releasing factor receptor signaling pathway, Cysteine biosynthesis, Cytoskeletal regulation by Rho GTPase, De novo purine biosynthesis, De novo pyrimidine deoxyribonucleotide biosynthesis, De novo pyrimidine ribonucleotides biosythesis, DNA replication, the Dopamine receptor mediated signaling pathway, the DPP-SCW signaling pathway, the DPP signaling pathway, the EGF receptor signaling pathway, the Endogenous cannabinoid signaling, the Endothelin signaling pathway, Enkephalin release, the FAS signaling pathway, the FGF signaling pathway, Flavin biosynthesis, Tetrahydrofolate biosynthesis, Formyltetrahydroformate biosynthesis, Fructose galactose metabolism, GABA-B receptor II signaling, Gamma-aminobutyric acid synthesis, the GBB signaling pathway, General transcription by RNA polymerase I, General transcription regulation, Glutamine glutamate conversion, Glycolysis, the Gonadotropin-releasing hormone receptor pathway, the Hedgehog signaling pathway, Heme biosynthesis, the Heterotrimeric G-protein signaling pathway-Gi alpha and Gs alpha mediated pathway, the Heterotrimeric G-protein signaling pathway-Gq alpha and Go alpha mediated pathway, Heterotrimeric G-protein signaling pathway-rod outer segment phototransduction, the Histamine H1 receptor mediated signaling pathway, the Histamine H2 receptor mediated signaling pathway, Histamine synthesis, Histidine biosynthesis, the Huntington disease pathway, Hypoxia response via HIF activation, the Inflammation mediated by chemokine and cytokine signaling pathway, Insulin/IGF pathway-mitogen activated protein kinase kinase/MAP kinase cascade, Insulin/IGF pathway-protein kinase B signaling cascade, the Integrin signalling pathway, the Interferon-gamma signaling pathway, the Interleukin signaling pathway, the Ionotropic glutamate receptor pathway, Isoleucine biosynthesis, the JAK/STAT signaling pathway, Leucine biosynthesis, Lipoate_biosynthesis, Lysine biosynthesis, Mannose metabolism, the Metabotropic glutamate receptor group III pathway, the Metabotropic glutamate receptor group II pathway, the Metabotropic glutamate receptor group I pathway, Methionine biosynthesis, Methylcitrate cycle, the Methylmalonyl pathway, mRNA splicing, the Muscarinic acetylcholine receptor 1 and 3 signaling pathway, the Muscarinic acetylcholine receptor 2 and 4 signaling pathway, the MYO signaling pathway, N-acetylglucosamine metabolism, Nicotine degradation, the Nicotine pharmacodynamics pathway, the Nicotinic acetylcholine receptor signaling pathway, the Notch signaling pathway, O-antigen biosynthesis, the Opioid prodynorphin pathway, the Opioid proenkephalin pathway, the Opioid proopiomelanocortin pathway, Ornithine degradation, Oxidative stress response, the Oxytocin receptor mediated signaling pathway, the p38 MAPK pathway, the p53 pathway, p53 pathway by glucose deprivation, P53 pathway feedback loops 1, p53 pathway feedback loops 2, Pantothenate biosynthesis, Parkinson disease, the PDGF signaling pathway, the Pentose phosphate pathway, Peptidoglycan biosynthesis, Phenylacetate degradation, Phenylalanine biosynthesis, Phenylethylamine degradation, Phenylpropionate degradation, the PI3 kinase pathway, Plasminogen activating cascade, Pyridoxal-5-phosphate biosynthesis, Proline biosynthesis, PRPP biosynthesis, Purine metabolism, the Pyridoxal phosphate salvage pathway, Pyrimidine Metabolism, Pyruvate metabolism, the Ras Pathway, S-adenosylmethionine biosynthesis, Salvage pyrimidine deoxyribonucleotides, Salvage pyrimidine ribonucleotides, the SCW signaling pathway, Serine glycine biosynthesis, Succinate to proprionate conversion, Sulfate assimilation, Synaptic vesicle trafficking, TCA cycle, the T cell activation pathway, the TGF-beta signaling pathway, Thiamin biosynthesis, Thiamin metabolism, Threonine biosynthesis, the Thyrotropin-releasing hormone receptor signaling pathway, the Toll pathway, the Toll receptor signaling pathway, Transcription regulation by bZIP transcription factor, Triacylglycerol metabolism, Tryptophan biosynthesis, Tyrosine biosynthesis, the Ubiquitin proteasome pathway, Valine biosynthesis, Vasopressin synthesis, the VEGF signaling pathway, Vitamin B6 biosynthesis, Vitamin B6 metabolism, the Vitamin D metabolism and pathway, the Wnt signaling pathway, the Xanthine and guanine salvage pathway, and the like.

Further non-limiting examples of signaling pathways, and description thereof, include the following: AKT Signaling Pathway (AKT is a serine/threonine kinase that is involved in mediating various biological responses, such as inhibition of apoptosis), Angiopoietin-TIE2 Signaling (The angiopoietins are a new family of growth factor ligands that bind to TIE2/TEK RTK (Receptor Tyrosine Kinase)), Antigen Processing and Presentation by MHCs (Antigen processing and presentation are the processes that result in association of proteins with major histocompatibility complex (MHC) molecules for recognition by a T-cell), Apoptosis Through Death Receptors (Certain cells have unique sensors, termed death receptors (DRs), which detect the presence of extracellular death signals and rapidly ignite the cell's intrinsic apoptosis machinery), APRIL Pathway (In immune responses, APRIL acts as a co-stimulator for B-cell and T-cell proliferation and supports class switch), B-Cell Development Pathway (The B-cell receptor (BCR) complex usually consists of an antigen-binding subunit that is composed of two Ig heavy chains, two Ig light chains, and a signaling subunit), BMP Pathway (Bone morphogenetic proteins (BMPs) are a large subclass of the transforming growth factor-beta (TGF-beta) superfamily), Cancer Immunoediting (The immune system attempts to constrain tumor growth, but sometimes tumor cells might escape or attenuate this immune pressure), CCR5 Pathway in Macrophages (C-C motif chemokine receptor type 5 (CCR5) is a member of the chemokine receptor subclass of the G protein-coupled receptor (GPCR) superfamily), CD4 and CD8 T-Cell Lineage (Each mature T-cell generally retains expression of the co-receptor molecule (CD4 or CD8) that has a major histocompatibility complex (MHC)-binding property that matches that of its T-cell receptor (TCR)), Cellular Apoptosis Pathway (Apoptosis is a naturally occurring process by which a cell is directed to programmed cell death), CTL-Mediated Apoptosis (The cytotoxic T lymphocytes (CTLs), also known as killer T-cells, are produced during cell-mediated immunity designed to remove body cells displaying a foreign epitope), CTLA4 Signaling Pathway (The co-stimulatory CTLA4 pathway attenuates or down-regulates T-cell activation CTLA4 is designed to remove body cells displaying a foreign epitope), Cytokine Network (Cytokines have been classified on the basis of their biological responses into pro- or anti-inflammatory cytokines, depending on their effects on immunocytes), ErbB Family Pathway (The ErbB family of transmembrane receptor tyrosine kinases (RTKs) plays an important role during the growth and development of organs), Fas Signaling (FAS (also called APO1 or CD95) is a death domain-containing member of the tumor necrosis factor (TNF) receptor superfamily), FGF Pathway (One of the most well characterized modulators of angiogenesis is the heparin-binding fibroblast growth factor (FGF)), Granulocyte Adhesion and Diapedesis (Adhesion and diapedesis of granulocytes have mostly been analyzed in context to non-lymphoid endothelium), Granzyme Pathway (Granzyme A (GzmA) activates a caspase-independent cell death pathway with morphological features of apoptosis), GSK3 Signaling (GSK3 is a ubiquitously expressed, highly conserved serine/threonine protein kinase found in all eukaryotes), Hematopoiesis from Multipotent Stem Cells (Hematopoietic stem cells are classified into long-term, short-term and multipotent progenitors, based on the extent of their self-renewal abilities), Hematopoiesis from Pluripotent Stem Cells (Pluripotent stem cells are capable of forming virtually all of the possible tissue types found in human beings), IL-2 Gene Expression in Activated and Quiescent T-Cells (IL-2 is a cytokine that stimulates the growth, proliferation, and differentiation of T-cells, B-cells, NK cells, and other immune cells), IL-6 Pathway (IL-6 is a pleiotropic cytokine that affects the immune system and many physiological events in various organs), IL-10 Pathway (IL-10 is a pleiotropic cytokine with important immunoregulatory functions and whose activities influence many immune cell types), IL-22 Pathway (IL-22 is a member of the IL-10 family of cytokines and exerts multiple effects on the immune system), Interferon Pathway (Interferons are pleiotropic cytokines best known for their ability to induce cellular resistance to viral infection), JAK/STAT Pathway (The JAK/STAT pathway is a signaling cascade whose evolutionarily conserved roles include cell proliferation and hematopoiesis), MAPK Family Pathway (Mitogen-activated protein kinases (MAPKs) belong to a large family of serine/threonine protein kinases that are conserved in organisms as diverse as yeast and humans), Nanog in Mammalian ESC Pluripotency (NANOG is a transcription factor transcribed in pluripotent stem cells and is down-regulated upon cell differentiation), p53-Mediated Apoptosis Pathway (Tumor protein p53 is a nuclear transcription factor that regulates the expression of a wide variety of genes involved in apoptosis, growth arrest, or senescence in response to genotoxic or cellular stress), Pathogenesis of Rheumatoid Arthritis (Rheumatoid arthritis (RA) is a chronic symmetric polyarticular joint disease that primarily affects the small joints of the hands and feet), PI3K Signaling in B Lymphocytes (The phosphoinositide 3-kinases (PI3Ks) regulate numerous biological processes, including cell growth, differentiation, survival, proliferation, migration, and metabolism), RANK Pathway (RANKL and its receptor RANK are key regulators of bone remodeling, and are essential for the development and activation of osteoclasts), RANK Signaling in Osteoclasts (RANKL induces the differentiation of osteoclast precursor cells and stimulates the resorption function and survival of mature osteoclasts), TGF-Beta Pathway (Members of the transforming growth factor (TGF)-beta family play an important role in the development, homeostasis, and repair of most tissues), THC Differentiation Pathway (T-helper cells of type 1 (TH1) and type 2 (TH2) are derived from T-helper cells and provide help to cells of both the innate and adaptive immune systems), TNF Signaling Pathway (Tumor necrosis factor (TNF) is a multifunctional pro-inflammatory cytokine with effects on lipid metabolism, coagulation, insulin resistance, and endothelial function), TNF Superfamily Pathway (The tumor necrosis factor (TNF) superfamily consists of 19 members that signal through 29 receptors that are members of the TNF receptor (TNFR) superfamily), Transendothelial Migration of Leukocytes (Transport of plasma proteins and solutes across the endothelium involves two different routes: transcellular and paracellular junctions), Tumoricidal Effects of Hepatic NK Cells (The liver is a major site for the formation and metastasis of tumors), TWEAK Pathway (TWEAK is a cell surface-associated protein belonging to the tumor necrosis factor (TNF) superfamily and has multiple biological activities), VEGF Family of Ligands and Receptor Interactions (Vascular endothelial growth factor (VEGF) is a highly-conserved genetic pathway that has evolved from simple to complex systems), and the like.

As summarized above, a component of a signaling pathway, including but not limited to a pathway described herein, may be modified to include a latent deactivation domain such that deactivation of the signaling pathway member may be controlled by expression of a switch polypeptide. Suitable pathway components that may be employed include e.g., input-receiving members, intermediate members, and output-producing members, including but not limited to e.g., the corresponding member of the pathways listed above.

Similarly, essentially any synthetic pathway may modulated using a molecular circuit as described herein. Suitable non-limiting examples of synthetic signaling pathways that may be modulated using a circuit of the present disclosure include, but are not limited to, those pathways controlled by a synthetic or engineered receptor, such as but not limited to e.g., a CAR, an engineered TCR, a synNotch, etc.

In some instances, a pathway modulated using a circuit of the present disclosure may include an immune modulation pathway, such as e.g., an immune activation pathway or an immune suppression pathway. Such immune modulation pathways may be natural or synthetic and may be endogenous to the cell in which the circuit is employed or heterologous to the cell in which the circuit is employed.

Suitable non-limiting examples of synthetic signaling pathways that may be modulated using a circuit of the present disclosure also include biosynthesis and/or bioproduction pathways. Biosynthesis and/or bioproduction pathways may be natural or synthetic and may be employed in cells and/or organisms where the pathway is endogenous or heterologous.

Non-limiting examples of biosynthesis pathways that may be modulated using a circuit of the present disclosure include, but are not limited to, hormone production pathways (e.g., an insulin production pathway, an estrogen/progesterone production pathway, an androgen production pathway, a growth hormone production pathway, and the like), opioid production pathways, isobutanol production pathways, non-ribosomal polyketide synthetase (NRPS) production pathways, antibiotic production pathways, chemotherapeutic production pathways, artemisinic acid production pathways, terpenoid production pathways, polyketide production pathways, and the like.

Non-limiting examples of synthetic biosynthesis pathways include but are not limited to e.g., synthetic hormone production pathways, synthetic opioid production pathways, synthetic antibiotic production pathways, synthetic chemotherapeutic production pathways, synthetic artemisinic acid production pathways, synthetic terpenoid production pathways, synthetic polyketide production pathways, and the like.

Nucleic Acids

As summarized above, the present disclosure also provides nucleic acids encoding molecular feedback circuits. The subject nucleic acids may include, e.g., a sequence encoding a switch polypeptide, sequence encoding a signaling protein that includes a latent deactivation domain, and the like. Such nucleic acids may be configured such that one or more of the sequences are operably linked to a regulatory sequence. For example, a nucleic acid may be configured such that the sequence encoding the switch polypeptide is operably linked to a regulatory sequence responsive to an output of the signaling pathway. Provided are nucleic acids encoding essentially any circuit employing a latent deactivation domain, including but not limited to those circuits specifically described herein. Encompassed are isolated nucleic acids encoding the subject circuits as well as various configurations containing such nucleic acids, such as vectors, e.g., expression cassettes, recombinant expression vectors, viral vectors, and the like.

Recombinant expression vectors of the present disclosure include those comprising one or more of the described nucleic acids. A nucleic acid comprising a nucleotide sequence encoding all or a portion of the components of a circuit of the present disclosure will in some embodiments be DNA, including, e.g., a recombinant expression vector. A nucleic acid comprising a nucleotide sequence encoding all or a portion of the components of a circuit of the present disclosure will in some embodiments be RNA, e.g., in vitro synthesized RNA.

As summarized above, in some instances, the subject circuits may make use of an encoding nucleic acid (e.g., a nucleic acid encoding a switch polypeptide or a latent deactivation domain-linked signaling protein) that is operably linked to a regulatory sequence such as a transcriptional control element (e.g., a promoter; an enhancer; etc.). In some cases, the transcriptional control element is inducible. In some cases, the transcriptional control element is constitutive. In some cases, the promoters are functional in eukaryotic cells. In some cases, the promoters are functional in prokaryotic cells. In some cases, the promoters are cell type-specific promoters. In some cases, the promoters are tissue-specific promoters.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., the cell cycle, the hair follicle cycle in mammals, circadian cycles in mammals, etc.).

Suitable promoter and enhancer elements are known in the art. For expression in a bacterial cell, suitable promoters include, but are not limited to, lacI, lacZ, T3, T7, gpt, lambda P and trc. For expression in a eukaryotic cell, suitable promoters include, but are not limited to, yeast promoters (e.g., promoters of yeast mating pathway genes, yeast galactose-inducible promoters, etc.), light and/or heavy chain immunoglobulin gene promoter and enhancer elements; cytomegalovirus immediate early promoter; herpes simplex virus thymidine kinase promoter; early and late SV40 promoters; promoters present in long terminal repeats from a retrovirus; mouse metallothionein-I promoter; and various art-known tissue specific promoters.

In some instances, transcriptional control elements of varied strength may be employed.

For example, promoters, e.g., constitutive or inducible promoters, of varied strength, such as e.g., weak, intermediate, and strong promoters, such as but not limited to e.g., constitutive promoters pREV1, pRNR2, pRET2, etc. may be employed. In some instances, the strength of a promoter may be modulated, e.g., made weaker or made stronger, by decreasing or increasing, respectively, the number of binding sites (e.g., DBD binding sites) within the promoter. Accordingly, the number of binding sites present in a subject promoter may vary and may range from 1 to 6 or more, including but not limited to e.g., 1, 2, 3, 4, 5, 6, etc.

In some instances, a transcriptional control element of a herein described nucleic acid may include a cis-acting regulatory sequence. Any suitable cis-acting regulatory sequence may find use in the herein described nucleic acids. For example, in some instances a cis-acting regulatory sequence may be or include an upstream activating sequence or upstream activation sequence (UAS). In some instances, a UAS of a herein described nucleic acid may be a Gal4 responsive UAS. In some instances, useful transcriptional control elements may include immune-related transcriptional control elements, such as but not limited to e.g., nuclear factor of activated T-cells (NFAT) promoters, and the like.

In some instances, transcriptional control of a circuit of the present disclosure may include the use of one or more regulatory elements responsive to a synthetic transcription factor. Synthetic transcription factors, and regulatory elements responsive thereto, will vary and may include but are not limited to e.g., estradiol ligand binding domain (LBD) based synthetic transcription factors, progesterone LBD based synthetic transcription factors, zinc-finger based synthetic transcription factors, and the like. Synthetic transcription factors may by chimeric and may include various domains, e.g., a DNA binding domain (DBD), activation domain, zinc-finger domain(s), and the like. Useful domains, e.g., LBDs, DBDs, activation domains, etc., will vary and may include but are not limited to e.g., the Gal4p DBD, the Zif268 transcription factor DBD, viral activation domains (e.g., VP16, VP64, etc.), Msn2p activation domains, and the like. Non-limiting examples of useful synthetic transcription factors include but are not limited to e.g., GEM (Gal4 DNA binding domain-Estradiol hormone binding domain-Msn2 activation domain), Z3PM (Z3 zinc finger-Progesterone hormone binding domain-Msn2 activation domain), and the like. Correspondingly, useful regulatory elements will vary and may include promoters responsive to synthetic transcription factors, including but not limited to e.g., pZ promoters, pZ3 promoters, pGAL1 promoters, and the like. Examples of suitable promoters and synthetic transcription factors include, but are not limited to e.g., those described herein, those described in Aranda-Diaz et al. ACS Synth Biol. (2017) 6(3): 545-554; the disclosure of which is incorporated herein by reference in its entirety, and the like.

Suitable promoters may, in some instances, include suitable reversible promoters.

Reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some instances, a useful promoter may be an immune cell promoter. For example, in embodiments were components of a circuit are expressed in an immune cell, an immune cell promoter may be employed. Suitable immune cell promoters include but are not limited to e.g., CD8 cell-specific promoters, CD4 cell-specific promoters, neutrophil-specific promoters, and NK-specific promoters. For example, a CD4 gene promoter can be used; see, e.g., Salmon et al. (1993) Proc. Natl. Acad. Sci. USA 90: 7739; and Marodon et al. (2003) Blood 101:3416. As another example, a CD8 gene promoter can be used. NK cell-specific expression can be achieved by use of an Ncrl (p46) promoter; see, e.g., Eckelhart et al. (2011) Blood 117:1565.

In some instances, an immune cell specific promoter of a nucleic acid of the present disclosure may be a promoter of a B29 gene promoter, a CD14 gene promoter, a CD43 gene promoter, a CD45 gene promoter, a CD68 gene promoter, a IFN-β gene promoter, a WASP gene promoter, a T-cell receptor β-chain gene promoter, a V9 γ (TRGV9) gene promoter, a V2 δ (TRDV2) gene promoter, and the like.

In some cases, a nucleic acid comprising a nucleotide sequence encoding a circuit of the present disclosure, or one or more components thereof, is a recombinant expression vector or is included in a recombinant expression vector. In some embodiments, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid comprising a nucleotide sequence encoding a circuit of the present disclosure, or one or more components thereof, is a recombinant lentivirus vector. In some cases, a nucleic acid comprising a nucleotide sequence encoding a circuit of the present disclosure, or one or more components thereof, is a recombinant AAV vector.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

In some instances, nucleic acids of the present disclosure may have a single sequence encoding two or more polypeptides where expression of the two or more polypeptides is made possible by the presence of a sequence element between the individual coding regions that facilitates separate expression of the individual polypeptides. Such sequence elements, may be referred to herein as bicistronic-facilitating sequences, where the presence of a bicistronic-facilitating sequence between two coding regions makes possible the expression of a separate polypeptide from each coding region present in a single nucleic acid sequence. In some instances, a nucleic acid may contain two coding regions encoding two polypeptides present in a single nucleic acid with a bicistronic-facilitating sequence between the coding regions. Any suitable method for separate expression of multiple individual polypeptides from a single nucleic acid sequence may be employed and, similarly, any suitable method of bicistronic expression may be employed.

In some instances, a bicistronic-facilitating sequence may allow for the expression of two polypeptides from a single nucleic acid sequence that are temporarily joined by a cleavable linking polypeptide. In such instances, a bicistronic-facilitating sequence may include one or more encoded peptide cleavage sites. Suitable peptide cleavage sites include those of self-cleaving peptides as well as those cleaved by a separate enzyme. In some instances, a peptide cleavage site of a bicistronic-facilitating sequence may include a furin cleavage site (i.e., the bicistronic-facilitating sequence may encode a furin cleavage site).

In some instances, the bicistronic-facilitating sequence may encode a self-cleaving peptide sequence. Useful self-cleaving peptide sequences include but are not limited to e.g., peptide 2A sequences, including but not limited to e.g., the T2A sequence.

In some instances, a bicistronic-facilitating sequence may include one or more spacer encoding sequences. Spacer encoding sequences generally encode an amino acid spacer, also referred to in some instances as a peptide tag. Useful spacer encoding sequences include but are not limited to e.g., V5 peptide encoding sequences, including those sequences encoding a V5 peptide tag.

Multi- or bicistronic expression of multiple coding sequences from a single nucleic acid sequence may make use of but is not limited to those methods employing furin cleavage, T2A, and V5 peptide tag sequences. For example, in some instances, an internal ribosome entry site (IRES) based system may be employed. Any suitable method of bicistronic expression may be employed including but not limited to e.g., those described in Yang et al. (2008) Gene Therapy. 15(21):1411-1423; Martin et al. (2006) BMC Biotechnology. 6:4; the disclosures of which are incorporated herein by reference in their entirety.

Cells

As summarized above, the present disclosure also provides cells containing nucleic acids encoding molecular feedback circuits. Cells modified to include one or more nucleic acids encoding one or more molecular feedback circuits and/or one or more components thereof may be referred to herein as having been genetically modified, where such modification may be stable or transient as desired. Useful cells may include prokaryotic and eukaryotic cells, including but not limited to e.g., bacterial cells, plant cells, animal cells, yeast cells, mammalian cells, rodent cells, non-human primate cells, human cells, and the like.

Suitable cells include stem cells, progenitor cells, as well as partially and fully differentiated cells. Suitable cells include, neurons, liver cells; kidney cells; immune cells; cardiac cells; skeletal muscle cells; smooth muscle cells; lung cells; and the like.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is a stem cell. In some cases, the cell is an induced pluripotent stem cell. In some cases, the cell is a mesenchymal stem cell. In some cases, the cell is a hematopoietic stem cell. In some cases, the cell is an adult stem cell.

Suitable cells include bronchioalveolar stem cells (BASCs), bulge epithelial stem cells (bESCs), corneal epithelial stem cells (CESCs), cardiac stem cells (CSCs), epidermal neural crest stem cells (eNCSCs), embryonic stem cells (ESCs), endothelial progenitor cells (EPCs), hepatic oval cells (HOCs), hematopoetic stem cells (HSCs), keratinocyte stem cells (KSCs), mesenchymal stem cells (MSCs), neuronal stem cells (NSCs), pancreatic stem cells (PSCs), retinal stem cells (RSCs), and skin-derived precursors (SKPs).

In some instances, a cell is an immune cell. Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or progenitor thereof, obtained from an individual. As another example, the cell is a cytotoxic cell, or progenitor thereof, obtained from an individual. As another example, the cell is a stem cell or progenitor cell obtained from an individual.

As used herein, the term “immune cells” generally includes white blood cells (leukocytes) which are derived from hematopoietic stem cells (HSC) produced in the bone marrow. “Immune cells” includes, e.g., lymphoid cells, i.e., lymphocytes (T cells, B cells, natural killer (NK) cells), and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), T-regulatory cells (Treg) and gamma-delta T cells. A “cytotoxic cell” includes CD8+ T cells, natural-killer (NK) cells, and neutrophils, which cells are capable of mediating cytotoxicity responses. “B cell” includes mature and immature cells of the B cell lineage including e.g., cells that express CD19 such as Pre B cells, Immature B cells, Mature B cells, Memory B cells and plasmablasts. Immune cells also include B cell progenitors such as Pro B cells and B cell lineage derivatives such as plasma cells.

Cells encoding a circuit of the present disclosure may be generated by any convenient method. Nucleic acids encoding one or more components of a subject circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

In some cases, the introduction of the subject nucleic acids and/or genetic modification is carried out ex vivo. For example, an immune cell, a stem cell, etc., is obtained from an individual; and the cell obtained from the individual is modified to express components of a circuit of the present disclosure. The modified cell can thus be modified with control feedback to one or more signaling pathways of choice, as defined by the one or more molecular feedback circuits present on the introduced nucleic acids. In some cases, the modified cell is modulated ex vivo. In other cases, the cell is introduced into (e.g., the individual from whom the cell was obtained) and/or already present in an individual; and the cell is modulated in vivo, e.g., by administering a nucleic acid or vector to the individual in vivo.

In some instances, cells employing a feedback circuit of the present disclosure may be therapeutic cells useful in cellular therapy of a subject. For example, in an application such as cellular therapy employing immune cells, the immune cells are engineered to deliver a therapeutic payload of interest in the human body. If the output of these engineered cells is too high, toxic effects may occur (such as e.g., cytokine release syndrome (CRS) as observed in CAR T cell therapies), but on the other hand an output that is too low then the therapy may be ineffective. Therapeutic cells can be fine-tuned to achieve a desired level of output (i.e., a setpoint) under well-controlled laboratory conditions. However, the dynamic environments in which engineered therapeutic cells function make guaranteeing that the output will remain constant over time difficult. Using the molecular circuits described herein for implementing feedback control, engineered cells have the ability to automatically correct against disturbances encountered the environment, including e.g., disturbances that cause the output to drift. In one aspect, self-regulating engineered cells are more robust in in vivo scenarios, thus improving existing cell therapy applications of synthetic biology.

In some instances, cellular therapeutics such as CAR T cells or synthetic receptor (e.g., SynNotch) enabled T cells greatly benefit from feedback control as a safety mechanism. A feedback controller in a CAR T cell may regulate the level of T cell activation and prevents toxic effects such as CRS which result from overstimulation of immune cells. Similarly, in SynNotch T cells, e.g., feedback control may enable delivery of a precise concentration of a payload of interest regardless of any disturbances to the engineered cell that are present or introduced. As will be readily understood, use of feedback control in therapeutic cells is not limited to these approaches and include other approaches as well.

Useful cells, within which circuits of the present disclosure may be employed, are not limited to therapeutic cells. For example, in some instances cells used in bioproduction may be employed. By “bioproduction”, as used herein, is generally meant processes by which a desired component is produced by cell for various applications, e.g., for industrial, commercial, biomedical, research, etc., applications. Biological products produced in bioproduction processes may vary and such products may be endogenous or heterologous to the cell and/or organism used in its production. In some instances, biological products of interest include, but are not limited to, recombinant therapeutic proteins, viruses (e.g. recombinant viruses for gene therapy), vaccines, antibodies, proteins and peptides (e.g., enzymes, growth factors, etc.), polysaccharides, nucleic acids (including DNA and RNA), cells, and nutritional products. Circuits and/or methods of the present disclosure may be used in conjunction with several different production techniques known in the art, such as the production of biological products using cells in a bioreactor (e.g., mammalian, yeast, bacteria, and/or insect cells), methods involving the use of transgenic animals (e.g. goats or chickens), methods involving the use of transgenic plants (e.g., tobacco, seeds or moss), and other methods known to those of skill in the art.

Where employed, suitable cells for bioproduction may include but are not limited to e.g., COS cells, NSO cells, SP2/0 cells, YB2/0 cells, and the like. Useful cells may be of prokaryotic (e.g., bacterial) or eukaryotic origin (including e.g., mammalian, yeast, plant, etc.) and may, in some instances, be established cell culture lines. Suitable cells may, in some instances, also include HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells. BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some instances, useful bioproduction cells may include yeast cells. Suitable yeast cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi. Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense. Fusarium sp., Fusarium gramineum, Fusarium venenatum, Neurospora crassa, Chlamydomonas reinhardtii, and the like.

In some instances, useful bioproduction cells may include prokaryotic cells. Suitable prokaryotic cells include, but are not limited to, any of a variety of laboratory strains of Escherichia coli, Lactobacillus sp., Salmonella sp., Shigella sp., and the like. See, e.g., Carrier et al. (1992) J. Immunol. 148:1176-1181; U.S. Pat. No. 6,447,784; and Sizemore et al. (1995) Science 270:299-302. Examples of Salmonella strains which can be employed include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter sphaeroides, Rhodobacter capsulatus, Rhodospirillum rubrum, Rhodococcus sp., and the like. In some embodiments, the cell is Escherichia coli.

In some instances, feedback control useful is cells employed for metabolic engineering, where the balance of enzymes in a metabolic pathway is essential to obtain an optimal titer of product. It is common for intermediates or even final products of metabolic pathways to have at least some level of toxicity to the host cell. Therefore, optimization of the ratios of enzymes is beneficial to maximizing the amount of product produced while maintaining effective cell growth. As an additional, due to the large size of reactors employed industrial fermentations, cells across a fermentation may experience highly variable environments and may be subjected to various different stressors at differing levels. These disturbances may cause the activity of enzymes to shift, necessitating “re-balancing” of pathway activity. A feedback controller employing a molecular circuit of the present disclosure mitigates the effects of disturbances, maximizing titers by dynamically rebalancing enzyme ratios.

Methods

As summarized above, the present disclosure also provides methods of using latent deactivation-based molecular feedback circuits. Such methods include but are not limited to e.g., methods of modulating a signaling pathway of a cell where the cell is or has been genetically modified with a latent deactivation-based molecular feedback circuit. Any of the above described circuits, and components thereof, may be employed in the herein described methods.

Various deactivation strategies may be employed to deactivate a signaling protein of a circuit employed in a method of the present disclosure. For example, in some instances, deactivation of the signaling protein may employ a degradation-based strategy, including e.g., where an employed deactivation domain results degradation of the signaling protein. In some instances, the deactivation domain may be or may include a degradation domain.

In some instances, deactivation of a signaling protein may employ a protease-based strategy, including e.g., where a latent deactivation domain is activated by a proteolytic cleavage event mediated by the switch polypeptide that is or includes a protease. In some instances, a signaling protein may be deactivated by an expressed protease, including but not limited to e.g., where a signaling protein is split by a protease.

In some instances, deactivation of a signaling protein may employ a localization-based strategy, including e.g., where deactivation of the signaling protein by the deactivation domain comprises re-localization of the signaling protein mediated by a switch polypeptide. In some instances, deactivation of the signaling protein by the deactivation domain includes sequestration of the signaling protein. In some instances, re-localization of the signaling protein may involve a binding event that associates a localization signal with the signaling protein. For example, in some instances, a deactivation domain may include a first member of a binding pair and a switch domain may include a second member of the binding pair linked to a sequestration domain.

In some instances, deactivation of a signaling protein may employ dominant negative suppression of the signaling protein, including e.g., where a switch polypeptide includes a dominant negative domain. For example, a subject method may include a split signaling protein reconstituted by binding of two members of a binding pair, where a switch polypeptide includes a member of a binding pair that includes or is linked to a dominant negative domain. In such methods, binding of the switch polypeptide may disrupt the re-association of the halves of the split signaling protein, thereby deactivating the split signaling protein. Accordingly, in some instances, dominant negative suppression of the signaling protein may include, but is not necessarily limited to, competitive binding of a non-covalently bound domain to a member of a binding pair linked to, or otherwise incorporated into, the signaling protein.

As described above, in some instances, useful members of a binding pair that may be employed in various methods of the present disclosure may include members of a leucine-zipper binding pair, i.e., the first and second members of a binding pair may include first and second portions of a leucine-zipper.

Methods employed for modulating signaling of a signaling pathway of a cell may serve various purposes. For example, in some instances, a circuit of the present disclosure may be employed in a method to provide feedback control of a signaling pathway of interest. In some instances, feedback control may include negative feedback control, which may, among other aspects, e.g., prevent the pathway from remaining active when a particular pathway output is produced and/or produced at or above a threshold level. In some instances, feedback control may include positive feedback control, which may, among other aspects, e.g., provide for amplification of a particular pathway output. In some instances, feedback control may provide for more stable output of a signaling pathway, including e.g., where the signaling output of the pathway is insulated from variables such as but not limited to e.g., environmental factors and inputs.

As described above, cells of the methods of the present disclosure may vary and may include in vitro and/or ex vivo cells genetically modified with one or more nucleic acids encoding one or more components of one or more circuits as described herein. In some instances, cells are primary cells obtained from a subject. In some instances, cells are obtained from a cell culture.

Accordingly, methods of the present disclosure may include obtaining cells used in the method, including where such cells are unmodified or have already been genetically modified to include a circuit of the present disclosure. In some instances, methods of the present disclosure may include performing the genetic modification. In some instances, methods of the present disclosure may include collecting cells, including where cells are collected before and/or after genetic modification. Methods of collecting cells may vary and may include e.g., collecting cells from a cell culture, collecting a cellular sample from a subject that includes the cells of interest, and the like.

In some instances, methods of the present disclosure may include modulating (e.g., increasing and/or decreasing) signaling of a signaling pathway, where such modulating involves activating a latent deactivation domain to cause deactivation of a signaling protein of the pathway. As described herein, the circuits of the present disclosure may include feedback, including positive and negative feedback. Feedback of the present methods may be dependent upon, at least in part, an output of the signaling pathway. Thus, once the circuit is initiated and/or a cell containing the circuit is delivered, modulation of the signaling pathway in accordance with the circuit may not necessitate further manipulation, i.e., feedback regulation of the signaling pathway by the circuit may be essentially automatic.

Accordingly, in methods employing cells that contain a molecular feedback circuit of the present disclosure, in some instances, the cells may be administered to the subject and no further manipulation of the circuit need be performed. For example, where a subject is treated with cells that contain a molecular feedback circuit of the present disclosure, the treatment may include administering the cells to the subject, including where such administration is the sole intervention to treat the subject.

In such methods, cells that may be administered may include, but are not limited to e.g., immune cells. In such methods, the circuit may be configured, in some instances, to modulate signaling of a native or synthetic signaling pathway of the immune cell, such as but not limited to e.g., an immune activation pathway or an immune suppression pathway. Non-limiting examples of suitable immune activation pathways, whether regulated by native or synthetic means, include cytokine signaling pathways, B cell receptor signaling pathways, T cell receptor signaling pathways, and the like. Non-limiting examples of suitable immune suppression pathways, whether regulated by native or synthetic means, include inhibitory immune checkpoint pathways, and the like.

Methods of the present disclosure may include administering to a subject, cells that express a therapeutic agent. Such cells may include a molecular feedback circuit of the present disclosure and may or may not be immune cells. For example, in some instances, a method may include administering to a subject a non-immune cell that produces a therapeutic agent, either endogenously or heterologously, where production of the therapeutic is controlled, in whole or in part, by the molecular feedback circuit. In some instances, a method may include administering to a subject an immune cell that produces a therapeutic agent, either endogenously or heterologously, where production of the therapeutic is controlled, in whole or in part, by the molecular feedback circuit. Non-limiting examples of suitable encoded therapeutic agents, include but are not limited to e.g., hormones or components of hormone production pathways, such as e.g., insulins or a component of an insulin production pathway, estrogen/progesterone or a component of an estrogen/progesterone production pathway, testosterone or a component of an androgen production pathway, growth hormone or a component of a growth hormone production pathway, or the like.

Such methods may be employed, in some instances, to treat a subject for a condition, including e.g., where the condition is a deficiency in a metabolic or a hormone. In such instances, the molecular feedback circuit may be configured such that the output of the molecular feedback circuit controls, in whole or in part, production and/or secretion of a metabolic or a hormone.

In some instances, the instant methods may include contacting a cell with one or more nucleic acids encoding a circuit wherein such contacting is sufficient to introduce the nucleic acid(s) into the cell. Any convenient method of introducing nucleic acids into a cell may find use herein including but not limited viral transfection, electroporation, lipofection, bombardment, chemical transformation, use of a transducible carrier (e.g., a transducible carrier protein), and the like. Nucleic acids may be introduced into cells maintained or cultured in vitro or ex vivo. Nucleic acids may also be introduced into a cell in a living subject in vivo, e.g., through the use of one or more vectors (e.g., viral vectors) that deliver the nucleic acids into the cell without the need to isolate, culture or maintain the cells outside of the subject.

Any convenient method of delivering the circuit encoding components may find use in the subject methods. In some instances, the subject circuit may be delivered by administering to the subject a cell expressing the circuit. In some instances, the subject circuit may be delivered by administering to the subject a nucleic acid comprising one or more nucleotide sequences encoding the circuit. Administering to a subject a nucleic acid encoding the circuit may include administering to the subject a cell containing the nucleic acid where the nucleic acid may or may not yet be expressed. In some instances, administering to a subject a nucleic acid encoding the circuit may include administering to the subject a vector designed to deliver the nucleic acid to a cell.

The subject methods may include introducing into a subject in need thereof, cells that contain nucleic acid sequences encoding a therapeutic, the expression of which is controlled, at least in part by a molecular feedback circuit. The therapeutic may be a therapeutic for the treatment of cancer. The introduced cells may be immune cells, including e.g., myeloid cells or lymphoid cells.

Non-limiting examples of cancers that may be treated include, e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma. etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, ect.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sézary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

In some instances, methods of the present disclosure may be employed to treat a subject for an immune dysfunction, including but not limited to e.g., where the condition is an autoimmune disease. For example, in some instances, a molecular feedback circuit of the present disclosure may be configured to regulate the immune activation level of a subject having an autoimmune disease, thus controlling the subject's autoimmune response to treat the subject for the autoimmune disease. In some instances, a subject having an autoimmune disease may be administered cells configured to contain a molecular feedback circuit of the present disclosure where the output of the molecular feedback circuit is immune suppression.

The present disclosure further includes methods of making the nucleic acids, circuits, and cells employed in the herein described methods. In making the subject nucleic acids and circuits, and components thereof, any convenient methods of nucleic acid manipulation, modification and amplification (e.g., collectively referred to as “cloning”) may be employed. In making the subject cells, containing the nucleic acids encoding the described circuits, convenient methods of transfection, transduction, culture, etc., may be employed.

A nucleotide sequence encoding all or a portion of the components of a circuit of the present disclosure can be present in an expression vector and/or a cloning vector. Where a subject circuit or component thereof is split between two or more separate polypeptides, nucleotide sequences encoding the two or more polypeptides can be cloned in the same or separate vectors. An expression vector can include a selectable marker, an origin of replication, and other features that provide for replication and/or maintenance of the vector. Suitable expression vectors include, e.g., plasmids, viral vectors, and the like.

Large numbers of suitable vectors and promoters are known to those of skill in the art; many are commercially available for generating a subject recombinant construct. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

As noted above, in some embodiments, a nucleic acid comprising a nucleotide sequence encoding a circuit or component thereof of the present disclosure will in some embodiments be DNA or RNA, e.g., in vitro synthesized DNA, recombinant DNA, in vitro synthesized RNA, recombinant RNA, etc. Methods for in vitro synthesis of DNA/RNA are known in the art; any known method can be used to synthesize DNA/RNA comprising a desired sequence. Methods for introducing DNA/RNA into a host cell are known in the art. Introducing DNA/RNA into a host cell can be carried out in vitro or ex vivo or in vivo. For example, a host cell (e.g., an NK cell, a cytotoxic T lymphocyte, etc.) can be transduced, transfected or electroporated in vitro or ex vivo with DNA/RNA comprising a nucleotide sequence encoding all or a portion of a circuit of the present disclosure.

Methods of the instant disclosure may further include culturing a cell genetically modified to encode a circuit of the instant disclosure including but not limited to e.g., culturing the cell prior to administration, culturing the cell in vitro or ex vivo (e.g., the presence or absence of one or more antigens), etc. Any convenient method of cell culture may be employed whereas such methods will vary based on various factors including but not limited to e.g., the type of cell being cultured, the intended use of the cell (e.g., whether the cell is cultured for research or therapeutic purposes), etc. In some instances, methods of the instant disclosure may further include common processes of cell culture including but not limited to e.g., seeding cell cultures, feeding cell cultures, passaging cell cultures, splitting cell cultures, analyzing cell cultures, treating cell cultures with a drug, harvesting cell cultures, etc.

Methods of the instant disclosure may, in some instances, further include receiving and/or collecting cells that are used in the subject methods. In some instances, cells are collected from a subject. Collecting cells from a subject may include obtaining a tissue sample from the subject and enriching, isolating and/or propagating the cells from the tissue sample. Isolation and/or enrichment of cells may be performed using any convenient method including e.g., isolation/enrichment by culture (e.g., adherent culture, suspension culture, etc.), cell sorting (e.g., FACS, microfluidics, etc.), and the like. Cells may be collected from any convenient cellular tissue sample including but not limited to e.g., blood (including e.g., peripheral blood, cord blood, etc.), bone marrow, a biopsy, a skin sample, a cheek swab, etc. In some instances, cells are received from a source including e.g., a blood bank, tissue bank, etc. Received cells may have been previously isolated or may be received as part of a tissue sample thus isolation/enrichment may be performed after receiving the cells and prior to use. In certain instances, received cells may be non-primary cells including e.g., cells of a cultured cell line. Suitable cells for use in the herein described methods are further detailed herein.

Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-[xxx] are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A molecular feedback circuit, the circuit comprising:

a signaling protein that, when activated by an input of a signaling pathway, drives an output of the signaling pathway, wherein the signaling protein comprises a latent deactivation domain; and

a regulatory sequence responsive to the output and operably linked to a nucleic acid sequence encoding a switch polypeptide that, when expressed, triggers the deactivation domain to deactivate the signaling molecule.
2. The circuit according to aspect 1, wherein the input, the output, or both comprise an intracellular signal.
3. The circuit according to aspect 1, wherein the input, the output, or both comprise an intercellular signal.
4. The circuit according to any of the preceding aspects, wherein the deactivation domain is a degradation domain.
5. The circuit according to aspect 4, wherein the degradation domain comprises a degron.
6. The circuit according to aspects 4 or 5, wherein the latent deactivation domain comprises a protection domain that prevents degradation of the signaling protein and is deprotected by the switch polypeptide.
7. The circuit according to aspect 6, wherein the switch polypeptide comprises a protease.
8. The circuit according to any of aspects 1 to 3, wherein the deactivation domain comprises a first member of a binding pair.
9. The circuit according to aspect 8, wherein the switch polypeptide comprises a second member of the binding pair linked to a sequestration domain.
10. The circuit according to aspect 9, wherein the sequestration domain comprises a plasma membrane-targeting tag, a mitochondrial membrane-targeting tag, a peroxisome-targeting tag, a vacuole-targeting tag, or an actin-cytoskeleton-targeting tag.
11. The circuit according to aspect 8, wherein the switch domain comprises a second member of the binding pair comprising a dominant negative domain.
12. The circuit according to aspect 11, wherein the latent deactivation domain comprises a competitive binding domain noncovalently bound to the first member of the binding pair.
13. The circuit according to any of aspects 8 to 12, wherein the first and second members of the binding pair comprise first and second portions of a leucine-zipper.
14. The circuit according to any of the preceding aspects, wherein the signaling protein is a positive regulator of the signaling pathway.
15. The circuit according to any of aspects 1 to 14, wherein the signaling protein is a negative regulator of the signaling pathway.
16. The circuit according to any of the preceding aspects, wherein the signaling protein is an intermediate member of the signaling pathway or a transcription factor.
17. The circuit according to aspect 16, wherein the transcription factor is a synthetic transcription factor.
18. The circuit according to any of the preceding aspects, wherein the regulatory sequence comprises a binding site for a transcription factor of the output.
19. The circuit according aspect 18, wherein the regulatory sequence comprises a plurality of binding sites for the transcription factor.
20. The circuit according to aspect 19, wherein the plurality of binding sites is 2 to 10 binding sites.
21. The circuit according to any of aspects 16 to 20, wherein the output is expression of the transcription factor.
22. The circuit according to any of aspects 1 to 15, wherein the signaling protein is a receptor and the input is a ligand for the receptor.
23. The circuit according to any of the preceding aspects, wherein the signaling pathway is selected from the group consisting of: a AKT signaling pathway, an Akt/PKB signaling pathway, an AMPK signaling pathway, an apoptosis signaling pathway, a BMP signaling pathway, a cAMP-dependent pathway, an estrogen signaling pathway. a hedgehog signaling pathway, a hippo signaling pathway, an immune activation pathway, an immune suppression pathway, an immune cell differentiation pathway, an insulin signal transduction pathway, a JAK-STAT signaling pathway, a MAPKIERK signaling pathway, a mTOR signaling pathway, an NF-KB signaling pathway, a nodal signaling pathway, a notch signaling pathway, a p53 signaling pathway, a PI3K signaling pathway, a TGF beta signaling pathway, a TLR signaling pathway, a TNF signaling pathway, a VEGF signaling pathway, and a Wnt signaling pathway.
24. The circuit according to any of the preceding aspects, wherein the circuit further comprises a regulatory sequence operably linked to a nucleic acid sequence encoding the signaling protein.
25. The circuit according to aspect 24, wherein the regulatory sequence operably linked to the nucleic acid sequence encoding the signaling protein is a native promoter of the signaling protein.
26. The circuit according to any of aspects 1 to 22, wherein the signaling pathway is a synthetic signaling pathway.
27. The circuit according to aspect 26, wherein the receptor is a synthetic receptor.
28. The circuit according to aspect 27, wherein the synthetic receptor is a synNotch receptor.
29. The circuit according to aspect 27, wherein the synthetic receptor is a chimeric antigen receptor (CAR) or an engineered T cell receptor (TCR).
30. The circuit according to aspect 29, wherein the output is immune activation or immune suppression.
31. One or more nucleic acid molecules encoding the molecular feedback circuit according to any of the preceding aspects.
32. A cell genetically modified to comprise the one or more nucleic acid molecules according to aspect 31.
33. The cell according to aspect 32, wherein the cell is a eukaryotic cell.
34. A method of treating a subject for a condition, the method comprising administering to the subject an effective amount of the eukaryotic cell according to aspect 33.
35. The method according to aspect 34, wherein the condition is a cancer and the output of the molecular feedback circuit is immune activation.
36. The method according to aspect 34, wherein the condition is an autoimmune disease and the output of the molecular feedback circuit is immune suppression.
37. The method according to aspect 34, wherein the condition is a deficiency in a metabolic or a hormone and the output of the molecular feedback circuit is production and/or secretion of the metabolic or the hormone.
38. A method of modulating signaling of a signaling pathway of a cell, the method comprising:

genetically modifying the cell with a molecular feedback circuit comprising:
a nucleic acid sequence encoding a signaling protein of the signaling pathway, the signaling protein comprising a latent deactivation domain; and
a regulatory sequence, responsive to an output of the signaling pathway, that is operably linked to a nucleic acid sequence encoding a switch polypeptide that, when expressed, activates the latent deactivation domain,

wherein the activated deactivation domain deactivates the signaling protein thereby modulating signaling of the signaling pathway.
39. The method according to aspect 38, wherein the modulating comprises negative feedback.
40. The method according to aspect 38, wherein the modulating comprises positive feedback.
41. The method according to any of aspects 38 to 40, wherein deactivation of the signaling protein by the deactivation domain comprises degradation of the signaling protein.
42. The method according to aspect 41, wherein the deactivation domain is a degradation domain.
43. The method according to aspects 41 or 42, wherein the latent deactivation domain is activated by a proteolytic cleavage event mediated by the switch polypeptide.
44. The method according to any of aspects 38 to 40, wherein deactivation of the signaling protein by the deactivation domain comprises sequestration of the signaling protein.
45. The method according to aspect 44, wherein the deactivation domain comprises a first member of a binding pair and the switch domain comprises a second member of the binding pair linked to a sequestration domain.
46. The method according to any of aspects 38 to 40, wherein deactivation of the signaling protein by the deactivation domain comprises dominant negative suppression of the signaling protein.
47. The method according to aspect 46, wherein the switch domain comprises a second member of the binding pair linked to a dominant negative domain.
48. The method according to aspects 46 or 47, wherein the latent deactivation domain comprises a competitive binding domain noncovalently bound to the first member of the binding pair.
49. The method according to any of aspects 45 to 48, wherein the first and second members of the binding pair comprise first and second portions of a leucine-zipper.
50. The method according to any of aspects 38 to 49, wherein the cell is an in vitro or ex vivo cell.
51. The method according to any of aspects 38 to 50, wherein the signaling pathway is a native signaling pathway of the cell.
52. The method according to aspect 51, wherein the native signaling pathway is a native biosynthesis pathway.
53. The method according to aspect 52, wherein the native biosynthesis pathway is a hormone production pathway.
54. The method according to aspect 53, wherein the hormone production pathway is selected from the group consisting of: an insulin production pathway, an estrogen/progesterone production pathway, an androgen production pathway, and a growth hormone production pathway.
55. The method according to aspect 44, wherein the cell is an immune cell and the native signaling pathway is an immune activation pathway or an immune suppression pathway.
56. The method according to aspect 48, wherein the immune activation pathway is selected from the group consisting of: a cytokine signaling pathway, a B cell receptor signaling pathway, and a T cell receptor signaling pathway.
57. The method according to aspect 48, wherein the immune suppression pathway is an inhibitory immune checkpoint pathway.
58. The method according to any of aspects 38 to 50, wherein the signaling pathway is a synthetic signaling pathway.
59. The method according to aspect 58, wherein the signaling protein is a synNotch receptor and the output is release of an intracellular domain of the synNotch receptor.
60. The method according to aspect 58, wherein the cell is an immune cell and the signaling pathway is a synthetic immune activation pathway or a synthetic immune suppression pathway.
61. The method according to aspect 60, wherein the immune cell is a myeloid cell or a lymphoid cell.
62. The method according to aspect 61, wherein the immune cell is a lymphoid cell selected from the group consisting of: a T lymphocyte, a B lymphocyte and a Natural Killer cell.
63. The method according to any of aspects 60 to 62, wherein the signaling protein is a synthetic immune receptor.
64. The method according to aspect 63, wherein the synthetic immune receptor is a chimeric antigen receptor (CAR) or an engineered T cell receptor (TCR).
65. The method according to any of aspects 58 to 64, wherein the output is immune activation or immune suppression.
66. The method according to aspect 58, wherein the synthetic signaling pathway is a synthetic biosynthesis pathway.
67. The method according to aspect 66, wherein the synthetic biosynthesis pathway is selected from the group consisting of: a hormone production pathway, an opioid production pathway, an antibiotic production pathway, a chemotherapeutic production pathway, an artemisinic acid production pathway, a terpenoid production pathway, and a polyketide production pathway.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Strategies Feedback Circuits Employing Latent Deactivation of a Component of Signaling Pathway

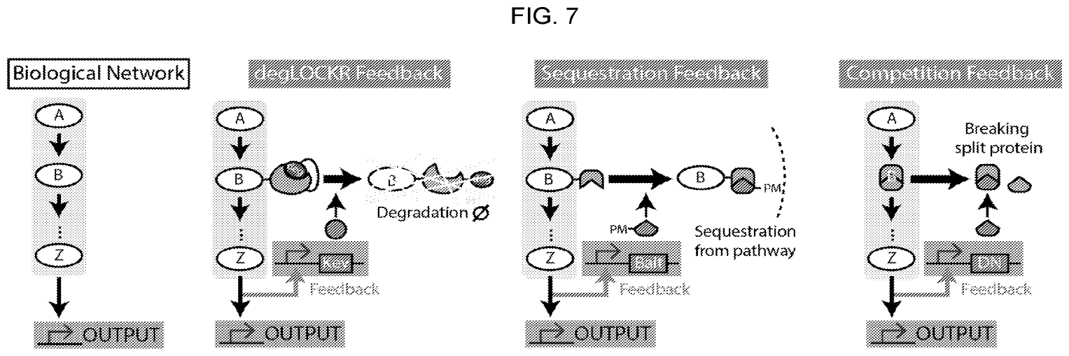

Various strategies were used to build feedback circuits that employ latent deactivation of a component of a signaling pathway. A schematized example of a signaling pathway, referred to as a “biological network” in these examples, is shown in the left panel of FIG. 7. As shown, components of the signaling pathway, signaling proteins “A”, “B” and “Z”, transduce an input to result in a transcriptional “output”. Examples of the employed strategies include induced degradation using degronLOCKR feedback (FIG. 7; 2^{nd} panel), sequestration feedback (FIG. 7; 3^{rd} panel), and competition feedback (FIG. 7, 4^{th} panel). In some instances, examples employing LOCKR-based systems (e.g., degronLOCKR, nesLOCKR, and nlsLOCKR), as described below, provide proof-of-concept demonstrations of general strategies of latent deactivation employed in the circuits described herein. However, in such examples the use of LOCKR-based systems is merely exemplary and/or comparative and may, in view of the instant disclosure, be readily substituted for LOCKR-independent systems (i.e., systems that do not employ LOCKR generally or, e.g., degronLOCKR, nesLOCKR, or nlsLOCKR specifically).

In the degronLOCKR feedback example, the circuit is configured such that a caged degron is attached to signaling protein “B”. The circuit is further configured to include a nucleic acid having a regulatory sequence operably linked to a sequence encoding a key polypeptide. As depicted, the regulatory sequence used is responsive to an intermediate component of the signaling pathway. Thus, when the signaling pathway transduces the signal and the intermediate component of the signaling pathway is expressed, or otherwise activated, the key polypeptide is expressed. Expression of the key polypeptide uncages the caged degron, resulting in degradation of signaling pathway component “B” and negative feedback on pathway signaling. Further examples of degronLOCKR feedback are provided in Example 4.

In the sequestration feedback example, the circuit is configured such that a first member of a binding pair, also referred to as “prey”, is attached to signaling protein “B”. The circuit is further configured to include a nucleic acid having a regulatory sequence operably linked to a sequence encoding a second member of the binding pair, referred to in FIG. 7 as “bait”, attached to a protein motif that localizes to the plasma membrane (PM). As depicted, the regulatory sequence used is responsive to an intermediate component of the signaling pathway. Thus, when the signaling pathway transduces the signal and the intermediate component of the signaling pathway is expressed, or otherwise activated, the “bait-PM” polypeptide is expressed. The expressed bait-PM polypeptide binds to the prey domain attached to signaling pathway component “B”. Accordingly, signaling pathway component “B” is sequestered to the plasma membrane, away from the intracellular location where component “B” functions within the signaling pathway. Thus, sequestration of component “B” results in negative feedback on pathway signaling. An example of sequestration feedback includes the “anchor away” circuit desribed in more detail below.

In the competition feedback example, the circuit is configured such that component “B” of the signaling pathway is split and the split portions reassociate due to leucine zipper domains incorporated into each of the split portions. Thus, the reconstituted split protein is capable of transducing signaling in the signaling pathway similar to the unsplit component “B” counterpart. The circuit is further configured to include a nucleic acid having a regulatory sequence operably linked to a sequence that encodes a dominant negative leucine zipper domain. The dominant negative leucine zipper domain binds to one or both of the component “B” leucine zipper domains with higher affinity than the affinity with which the component “B” leucine zipper domains bind. Thus, when present, the dominant negative leucine zipper domain outcompetes the reassociation of the split portions of the component “B”. Accordingly, signaling pathway component “B” is deactivated when the dominant negative leucine zipper domain is expressed, resulting in negative feedback on pathway signaling. An example of competition feedback includes the circuit desribed in Example 3 below.

### Example 2

### Anchor Away: A Sequestration-Based Feedback Circuit

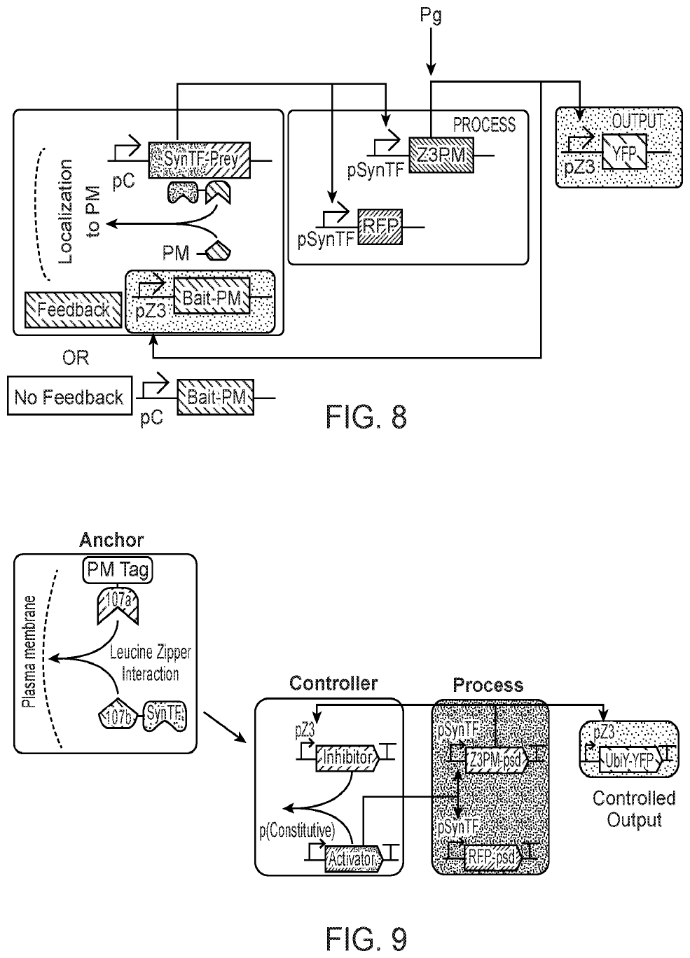

A sequestration-based feedback circuit, named “anchor away”, was configured as schematically depicted in FIG. 8. Specifically, synthetic transcription factor (“SynTF”) GEM was fused to one half of a leucine zipper (“prey”) and is induced by E2 to activate transcription from the pGAL1 promoter. GEM activates production of Z3PM and RFP as a sensor. Z3PM is induced by Pg to activate transcription from the pZ3 promoter. Z3PM activates production of YFP and the other half of a leucine zipper that is fused to a plasma membrane targeting domain (“bait-PM”). Feedback localizes GEM to the plasma membrane and prevents it from activating transcription. An alternate depiction of an anchor away circuit, denoting the “anchor”, “controller”, “process”, and “controlled output” portions of the circuit, is provided in FIG. 9.

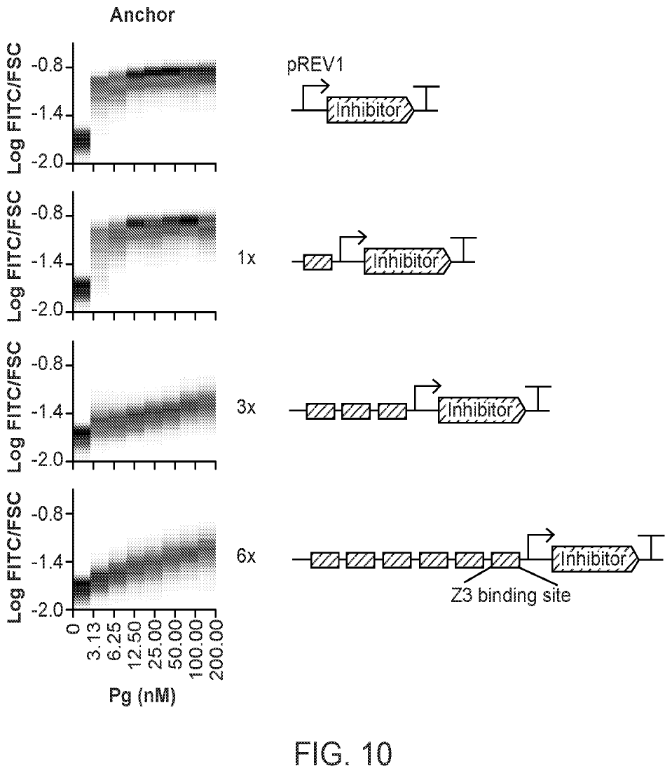

The anchor away feedback circuit was constructed and tested in conjunction with a corresponding “no feedback” control circuit using YFP to measure circuit output. In FIG. 10 steady-state YFP output as a function of progesterone is shown for anchor away feedback and behavior without feedback is shown in the top row. Increasing promoter strength (top to bottom) corresponds to increasing number of Z3 binding sites activating the inhibitor (i.e., bait). These data show that stronger promoters resulted in a greater feedback effect, resulting in lower maximum output and also a change in the slope of the shown feedback curves.

### Example 3

### Competition-Based Feedback

CCAAT/enhancer-binding protein alpha (CEBPα) is a leucine zipper transcription factor that dimerizes and activates the GCAAT promoter. “DN” is a dominant negative that binds the CEBPα monomer with high affinity, preventing the transcription factor from binding DNA and activating transcription (FIG. 11; see also Buchler & Cross. Molecular Systems Biology (2009) 5:272; the disclosure of which is incorporated herein by reference in its entirety).

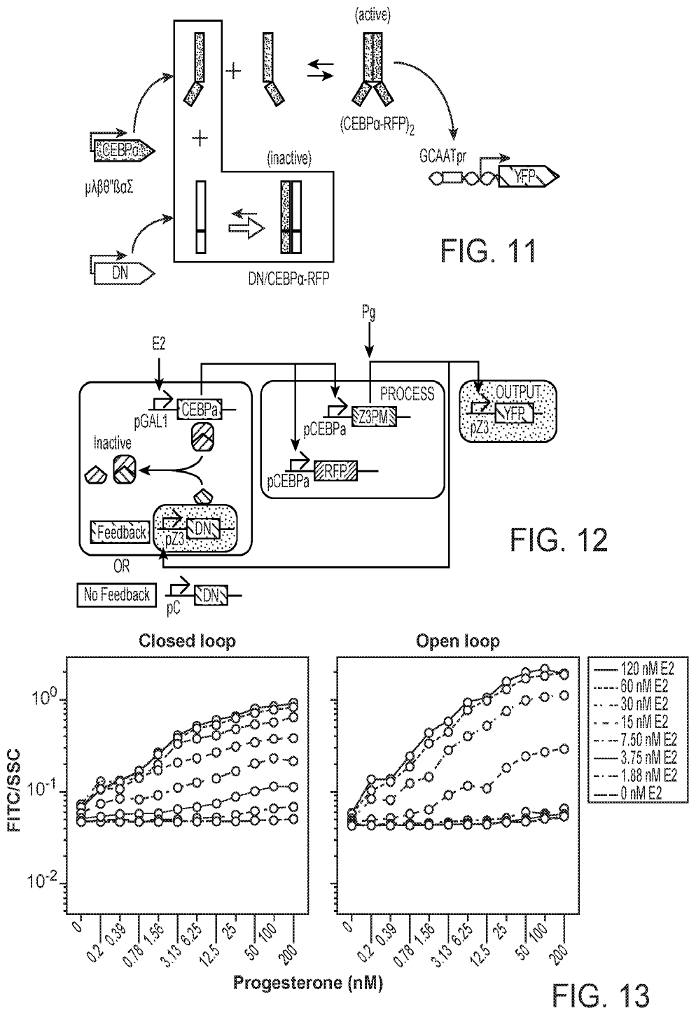

The anchor away feedback circuit was re-engineered to replace the sequestration-based feedback with components for competition-based feedback as schematically depicted in FIG. 12. GEM (and E2) determine the setpoint of the circuit via production of CEBPα. CEBPα activates transcription of Z3PM. Z3PM is induced by Pg to activate transcription from the pZ3 promoter. Z3PM activates production of YFP and the other half of the leucine zipper (DN) that splits apart the CEBPa TF dimer. Thus, feedback inactivates CEBPa and tunes down production of Z3PM as output increases.

The closed-loop feedback circuit depicted in FIG. 12 was constructed and compared to a corresponding open-loop feedback circuit using YFP to measure circuit output. In FIG. 13, steady-state YFP output as a function of progesterone is shown with feedback (left) and without feedback (right). As shown, increasing amounts of E2 increased the output of the circuit. Feedback decreased the maximum output of the circuit and also decreased the slope of the dependence on Pg. Collectively, the data shows that the closed-loop feedback circuit using the DN alters the slope of the Pg dose response in comparison to a corresponding circuit without feedback (open loop). This example demonstrates that dominant negative production can be used to implement negative feedback on leucine zipper transcription factor in a competition-based feedback circuit.

### Example 4

### Modular and Tunable Biological Feedback Control Using a De Novo Protein Switch

In this example, a de novo protein switch, degronLOCKR, designed via host-agnostic parts with modular connectivity and predictable tunability is employed to implement feedback control on endogenous pathways and synthetic circuits in the yeast S. cerevisiae.

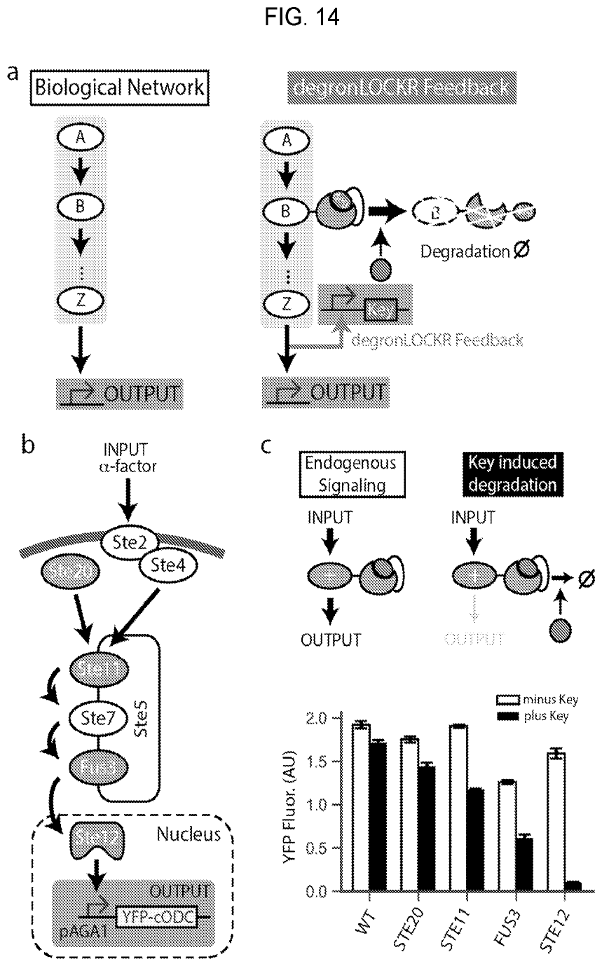

The degronLOCKR device is based on LOCKR (Latching Orthogonal Cage Key pRoteins) technology, and consists of the designer degSwitch and key proteins. The degSwitch is a six-helix bundle that has the cODC degron embedded in the destabilized sixth helix (latch), which is occluded via interaction with the five-helix scaffold (cage). The key, a genetically encoded peptide, can outcompete the latch for binding with the cage. This reveals the cODC degron, thus targeting the degSwitch and any fused cargo to the proteasome for degradation. degronLOCKR is a powerful device for synthetic biology because protein degradation is a universal method for post-translational regulation. It has been shown that degronLOCKR can control gene expression by regulating the stability of a transcription factor. Here, this functionality is capitalized on to implement modular feedback control on a biological network using degronLOCKR by expressing the key as a function of the output of the network (FIG. 14, panel a). The degronLOCKR feedback strategy offers several advantages over other approaches for implementing feedback control. First, the modular nature of the degronLOCKR allows the degSwitch to be directly fused to any protein of interest to generate on-target effects. Modifying endogenous genes with the degSwitch also preserves the native transcriptional and translational regulation of the signaling protein. Finally, degronLOCKR is a completely de novo designed protein thus allowing for predictable modifications to tune its characteristics.

degronLOCKR Synthetic Negative Feedback in Endogenous Yeast Pathway

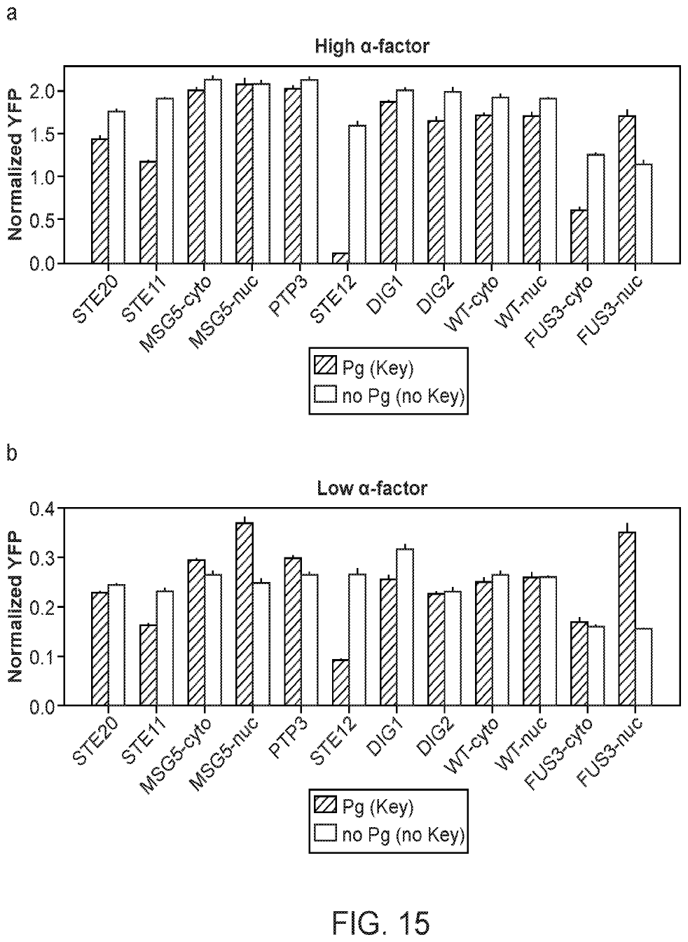

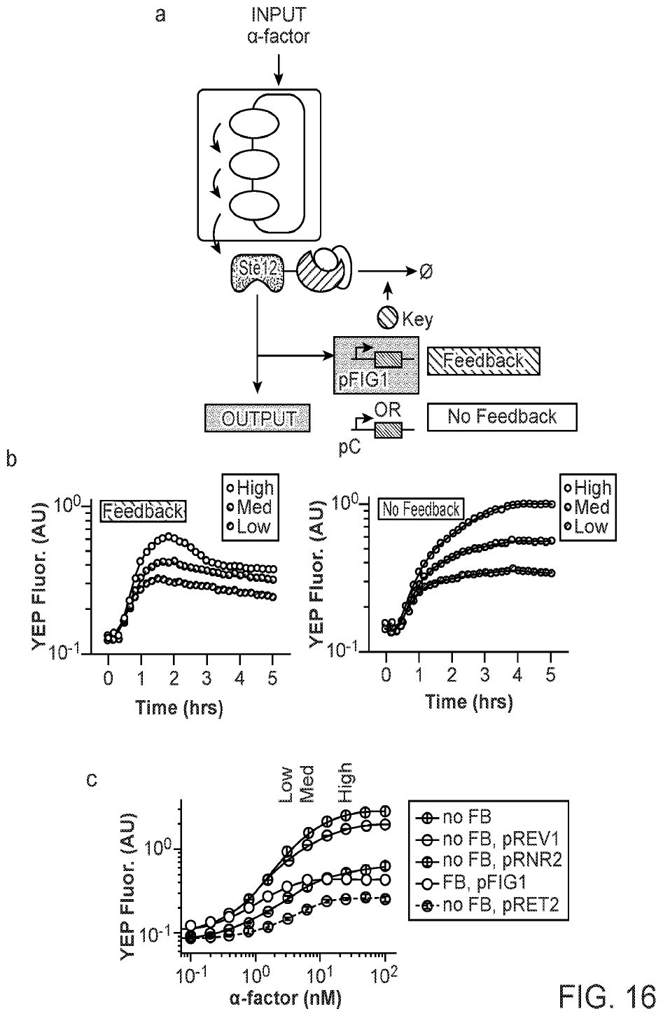

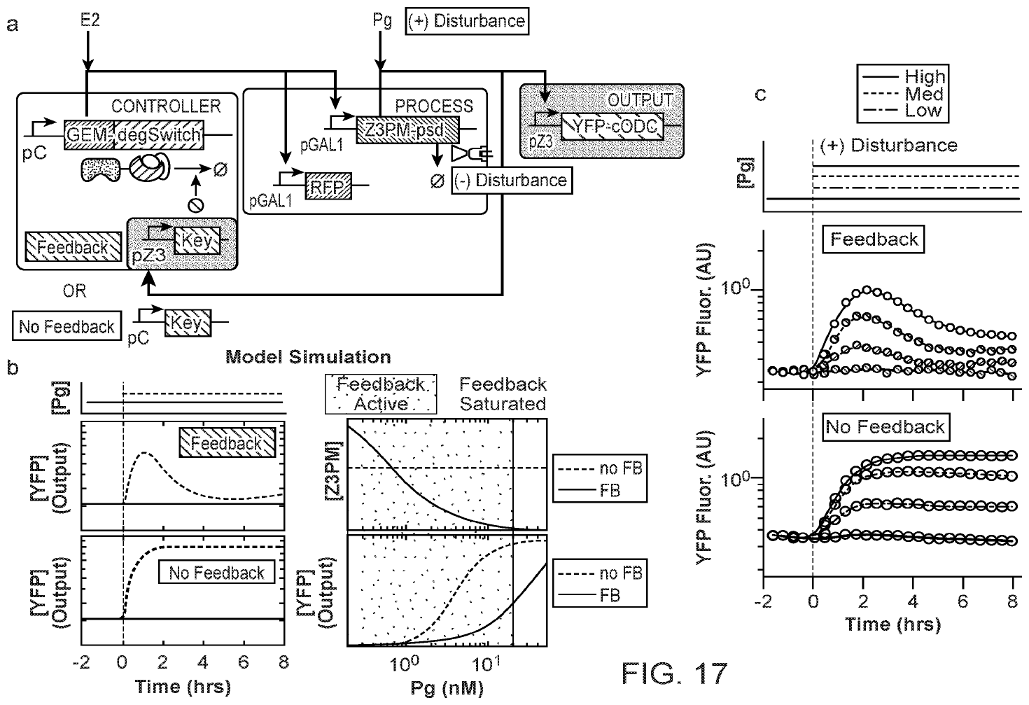

As a qualitative proof of concept, degronLOCKR was used to implement synthetic negative feedback in the yeast MAPK mating pathway (FIG. 14, panel b), a complex signaling pathway with many endogenous feedback loops. The ability of degronLOCKR to modulate pathway output was tested by appending the degSwitch to the endogenous locus of several positive pathway molecules in a ΔFAR1 ΔBAR1 background strain and the key was expressed using an inducible system (Aranda-Diaz et al. ACS Synth. Biol. 6, 545-554 (2017)) (FIG. 14, panel c). The key was targeted to either the cytosol or nucleus using a nuclear localization sequence to trigger degradation of each molecule in a specific compartment of the cell (FIG. 15). This localized inducible degradation is a unique characteristic of degronLOCKR that enables location specific action in the cell. The mating pathway was stimulated with a saturating dose of α-factor (100 nM) and monitored pathway activity using pAGA1-YFP-cODC (McCullagh et al. Nat. Cell Biol. 12, 954-962 (2010)) transcriptional reporter (cODC degron (Hoyt et al. J. Biol. Chem. 278, 12135-12143 (2003)) destabilizes the long lived fluorescent reporter, allowing dynamics to be observed). Degrading STE20 (MAPKKKK), STE11 (MAPKKK), and FUS3 (MAPK) had a moderate effect, while degrading STE12 (TF) completely eliminated the output of the mating pathway (FIG. 14, panel c, bottom). These data indicate that degronLOCKR is an effective tool for modulating endogenous pathways.

Synthetic negative feedback control of the mating pathway was next implemented by expressing the key-CFP-NLS from a mating pathway responsive promoter (pFIG1) in a strain where endogenous STE12 is fused to the degSwitch (FIG. 16, panel a). The effect of this feedback was compared to a strain with no feedback where STE12 is still fused to degSwitch but the key is driven by a constitutive promoter. pAGA1-YFP-cODC dynamics were followed after stimulation with high (25 nM), medium (6.25 nM), and low (3.13 nM) doses of α-factor (Fig FIG. 16, panel b) using automated flow cytometry. For comparison, a strain without feedback (pREV1-key-CFP-NLS) was simultaneously measured. Following stimulation with each dose of alpha-factor, the output of the synthetic feedback and no feedback strains initially followed each other closely. After around two hours, the synthetic feedback output started to decrease while the no feedback output increased to different steady-states corresponding to the different doses of α-factor. The strain with degronLOCKR synthetic feedback displayed larger transient overshoots for larger doses of α-factor, but eventually converged on the same steady-state output regardless of the input size. These data suggest that synthetic feedback desensitizes the steady-state output to α-factor of the mating pathway in this input regime. This effect is likely not due to saturation of signaling because of the different observed transients.

To obtain a more global comparison of the steady-state behaviors of the synthetic feedback and no feedback strains, the output dose response of each was measured as a function of α-factor. The feedback strain displayed attenuation of maximum output magnitude and decreased slope in the linear region of the dose response (FIG. 16, panel c). Comparing the synthetic feedback strain to no feedback strains with a range of constitutive promoter strengths (Lee et al. ACS Synth. Biol. 4, 975-986 (2015)) (pREV1, pRNR2, pRET2) indicates that the behavior generated by feedback cannot be achieved by expressing different constitutive amounts of the key. Taken together, the dynamic adaptation behavior and dose response clearly demonstrate the effect of synthetic negative feedback and utility of degronLOCKR as a tool for rapid rewiring of a complex endogenous signaling pathway.

degronLOCKR Feedback in a Synthetic Transcriptional Cascade

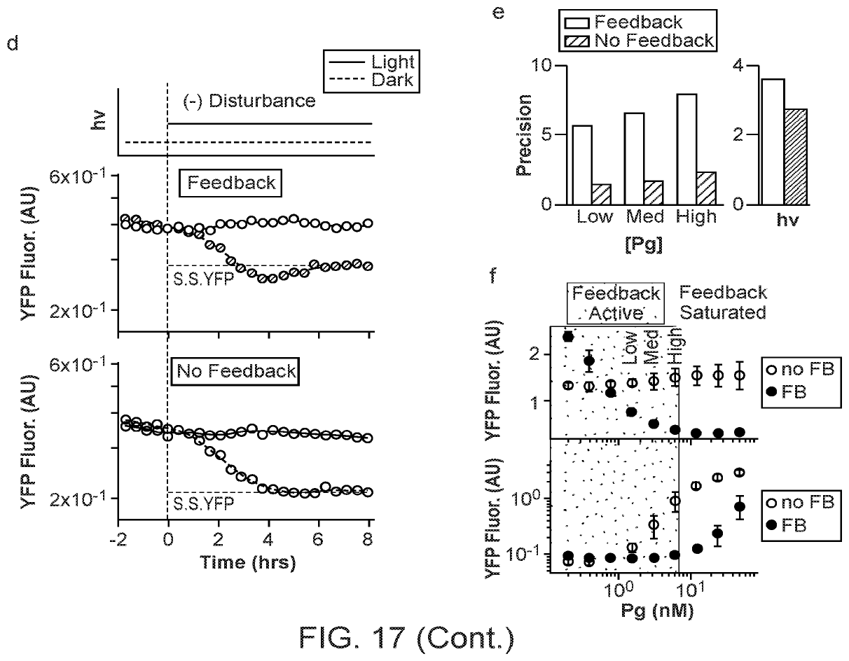

The quantitative capabilities and operational range of the degronLOCKR feedback module was next mapped using a simple synthetic transcriptional cascade consisting of two inducible synthetic transcription factors (Aranda-Diaz et al.) (FIG. 17, panel a). The first, GEM (Gal4 DNA binding domain-Estradiol hormone binding domain-Msn2 activation domain), is induced by estradiol (E2) and activates pGAL1 to produce Z3PM (Z3 zinc finger-Progesterone hormone binding domain-Msn2 activation domain). Z3PM, in turn, is induced by progesterone (Pg) and activates transcription of pZ3-YFP-cODC. To implement feedback, the same modular strategy that was successful for controlling the mating pathway was used: fusing GEM to the degSwitch and using pZ3 to express key-CFP-NLS (synthetic feedback). With feedback, the concentration of GEM is dependent on the output of Z3PM because the amount of key produced, and hence degradation rate of GEM, is a function of Z3PM activity. The circuit can be perturbed by addition of Pg or induction of a blue-light inducible degron (psd) (Renicke et al. Chem. Biol. 20, 619-626 (2013)) fused to Z3PM to increase or decrease the output, respectively. Feedback buffers against these disturbances by modulating the concentration of Z3PM. This type of disturbance rejection experiment is an essential test of feedback in technological systems.

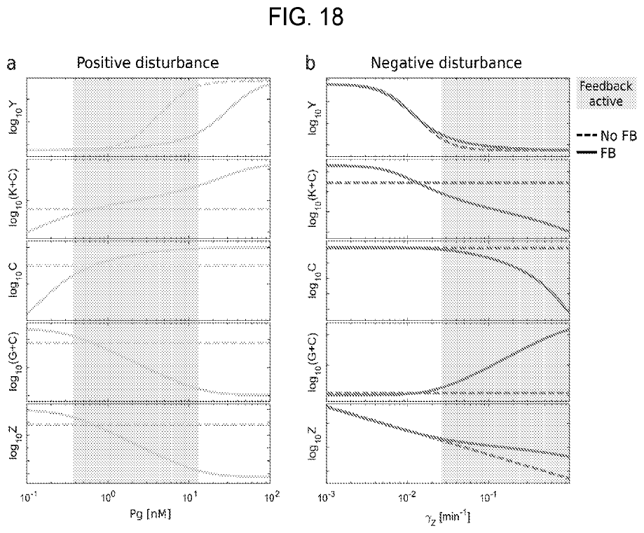

A simple computational model of the circuit predicts that an increase in Pg results in a monotonic increase in output without feedback (key expressed constitutively), whereas feedback gives a transient increase in output followed by adaptation to a steady-state whose value is closer to the pre-disturbance value than the circuit with no feedback for the same increase in Pg (FIG. 17, panel b, left). Feedback attenuates the dependence of the output on the Pg disturbance by decreasing the production rate of Z3PM, therefore compensating for an increase in Z3PM activity after a Pg increase with a decrease in its concentration (FIG. 17, panel b, right; FIG. 18).

These predictions were experimentally verified by first inducing cells with 7.5 nM E2 and 0.78 nM Pg and which were grown until their output reached steady-state (FIG. 17, panel c). At that time, the cells were perturbed with a high (6.25 nM), medium (3.13 nM), or low (1.56 nM) step-input of Pg and the dynamics of pZ3-YFP-cODC were measured using an automated flow cytometry and optogenetically-enabled continuous culture platform. As a control, the same series of inductions were performed on an strain without feedback (pRNR2 expressing key) which had similar YFP steady-state output as the feedback strain at the pre-disturbance concentration of E2 and Pg. Without feedback, the step-input of Pg caused an increase in Z3PM activity and thus YFP expression until the output reached a new steady-state commensurate with the disturbance. In contrast, the synthetic feedback circuit increased key expression as Z3PM activity increased, resulting in GEM degradation and thus a decrease in Z3PM production. This buffering effect is visible starting two hours post-disturbance when the synthetic feedback circuit output begins to decrease while the no feedback circuit output continues to climb. This adaptation behavior is qualitatively similar to the synthetic negative feedback loop constructed for the mating pathway. Because of the well-defined inputs and disturbances, adaptation can be quantified using a precision metric calculated by taking the inverse of the absolute difference between post- and pre-disturbance output normalized by the pre-disturbance output (Ma et al. Cell 138, 760-773 (2009)) (FIG. 17, panel e). The feedback circuit generates much higher precision than the circuit without feedback for the Pg positive disturbance, showcasing a benefits of feedback control.

A similar experiment was performed where the cells were subjected to a negative disturbance. Cells were grown to steady-state at 30 nM E2 and 1.57 nM Pg, then induced with blue light to activate degradation of Z3PM (FIG. 17, panel d). As a control, a no feedback circuit was built as a control with the key expressed constitutively from pRPL18B to match the steady-state expression of the synthetic feedback circuit before disturbance. After an immediate decrease in YFP expression in both synthetic feedback and no feedback circuits as a result of Z3PM degradation, the no feedback circuit settled to a new lower steady-state. The feedback circuit, however, underwent a slight overshoot after which it recovered to a steady-state closer to the pre-disturbance value than the no feedback circuit. The amount of adaptation in the synthetic feedback circuit for the negative disturbance is not as dramatic as for the positive disturbance (FIG. 17, panel f). Model simulation shows that the negative disturbance pushes the circuit output to a lower expression level where the relative difference between a circuit with and without feedback will be smaller Thus, even if feedback is still actively buffering against the negative disturbance the effect will be harder to observe. This underscores the fact that any feedback circuit, whether built with biological molecules or electronic components, has properties that need to be explored through thorough prototyping in order to enable productive modular use.

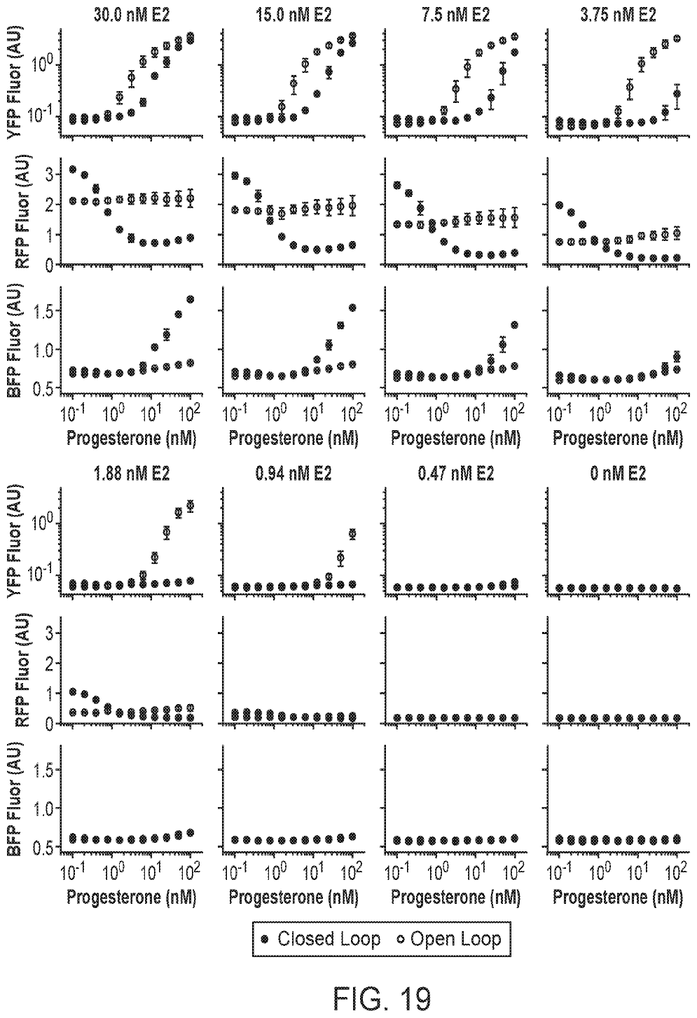

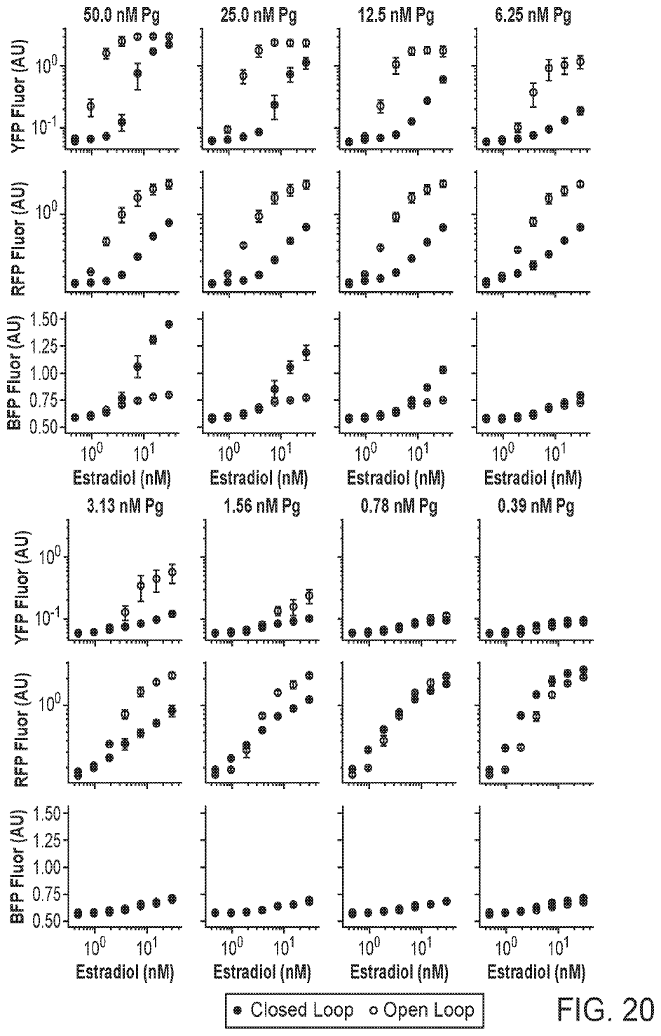

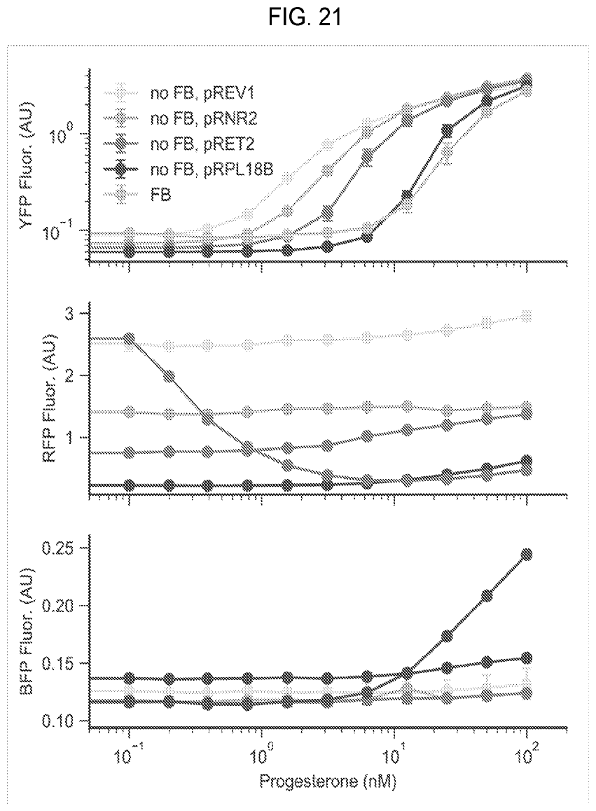

To further delineate the properties of the degronLOCK feedback module, the feedback and no feedback circuits were induced with the full range of E2 and Pg concentrations and measured pZ3-YFP-cODC output at steady-state using flow cytometry (FIG. 19 and FIG. 20). In these experiments, pGAL1-RFP was measured to gain more proximal information about the activity of GEM and thus the feedback action. At a fixed concentration of E2 (7.5 nM E2), increasing Pg leads to an increase in the YFP output of the no feedback circuit until saturation is reached (FIG. 21, FIG. 17. panel e). Because the key is expressed from a constitutive promoter in this strain, RFP fluorescence is insensitive to Pg. In contrast, RFP fluorescence decreases as a function of Pg in the synthetic feedback circuit, a result of degronLOCKR induced degradation of GEM. This effect eventually saturates above 6.25 nM Pg, as shown by the constant RFP expression beyond this concentration. The difference between these two regions of operation is clearly visible in the YFP output, which shows reduced sensitivity to Pg in the region of active feedback and a dramatic increase when feedback is saturated. These results are qualitatively recapitulated by the model, which shows the feedback saturating when the complex formation between the key and degSwitch saturates (FIG. 16 and FIG. 17).

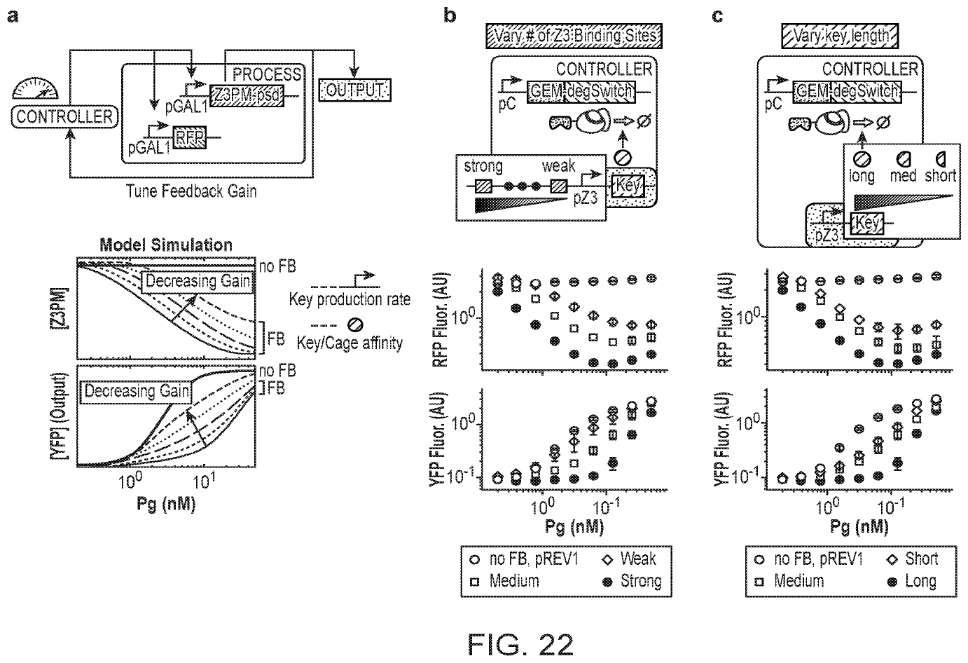

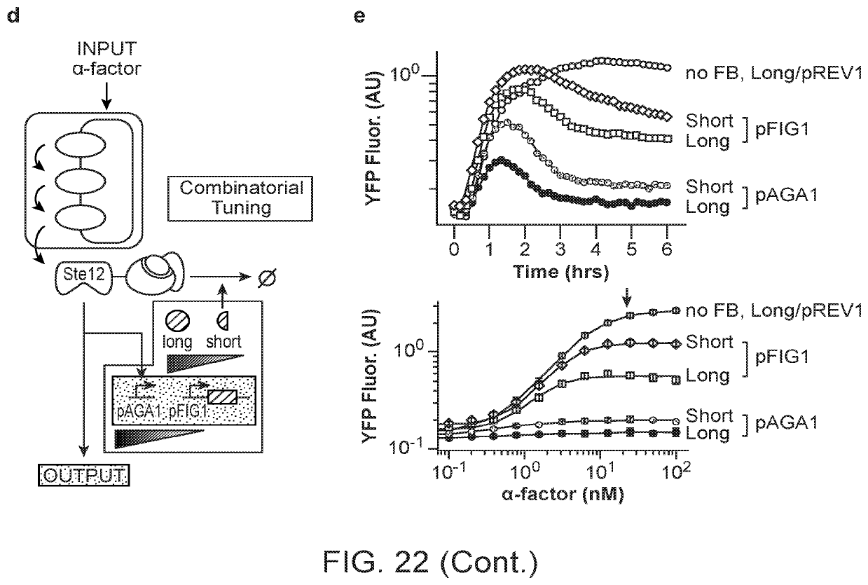

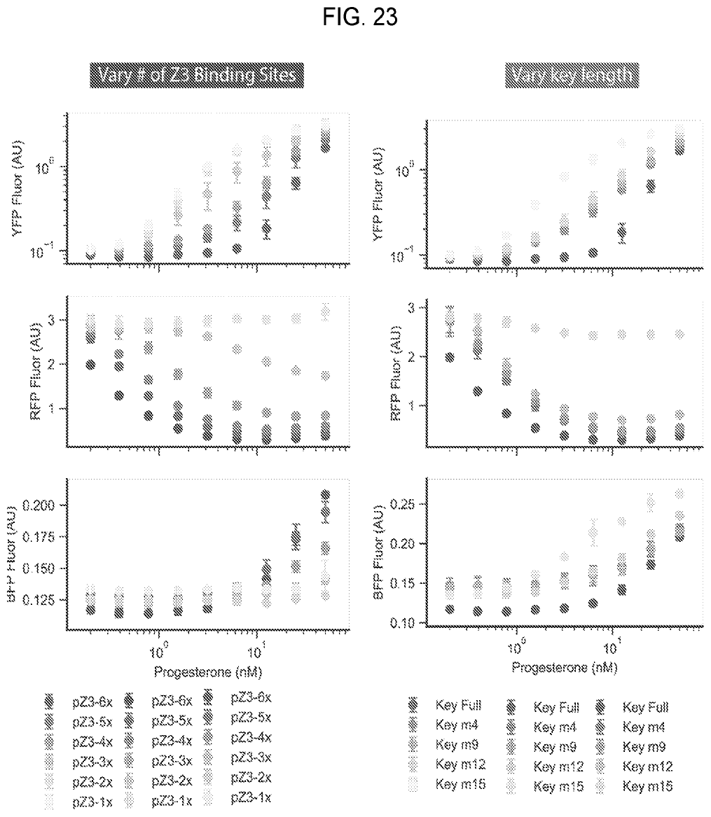

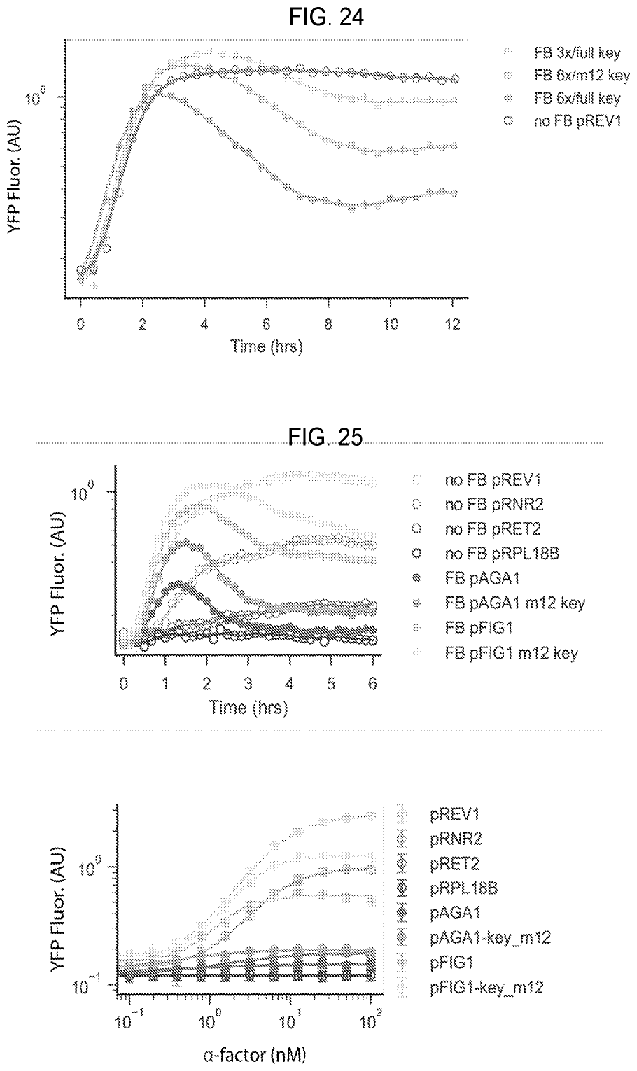

Next the tunability degronLOCKR feedback control was investigated. An useful aspect of designed feedback controllers is the ability to tune the gain to suit the application. Two methods of tuning feedback gain were evaluated: changing the strength of the feedback promoter and changing the binding affinity of the key and cage (FIG. 22, panel a). The computational model predicts that both methods for tuning feedback gain are qualitatively similar and thus should be interchangeable (FIG. 22, panel b). To test this, medium and weak variants of the pZ3 promoter with four and three Z3 binding sites (BS), respectively, were created. To test the effect of weakening the feedback promoter strength, a Pg dose response at a fixed concentration of E2 of the different circuit variants was performed (FIG. 22, panel b). It was observed that weakening the promoter indeed changed the dependence of the steady-state output on Pg. As the number of binding sites was reduced, the output dose response for the feedback circuit converged to the circuit without feedback (FIG. 23). Next, the affinity of the key for the cage was decreased by decreasing the length of the key. The full-length key was truncated by four (medium) or 12 (short) residues and each key variant was tested in the feedback circuit using the full-strength pZ3 promoter (6× Z3 BS) (FIG. 22, panel c, FIG. 23). Similar to reducing the strength of the feedback promoter, a decrease in key length led to a change in the dependence of the steady-state output on Pg (FIG. 22, panel d). Reducing the strength of the feedback gain through either strategy also led to larger transients and reduced adaptation (FIG. 24). Tuning feedback gain through key length is an attractive alternative to promoter engineering, showcasing a unique strength of de novo proteins.

To show the generalizability of these tuning strategies, the mating pathway was revised and combinatorial tuning of the synthetic feedback loop was performed by changing both the strength of the feedback promoter and the length of the key (FIG. 22, panel d). Because pAGAl is a much stronger promoter than pFIG1, using pAGAl to express the key generated a pulse of expression following induction with alpha-factor (FIG. 22, panel e). The size of the pulse, as well as the steady-state output following it were both increased by reducing the key length, which reduced the amount of feedback in the system. Similarly, reducing the key length while using the weaker promoter pFIG1 to drive feedback yielded a larger transient and higher steady-state output. Measurement of steady-state output as a function of a-factor for different promoters and key lengths (FIG. 22, panel f, FIG. 25) clearly demonstrates that reducing promoter strength or key length increases the steady-state output of the pathway and the slope of the dose response, indicating reduced feedback gain. Taken together, these data demonstrate the facile tunability of the characteristics of the degronLOCKR feedback strategy.

Methods

Construction of DNA Circuits

Hierarchical golden gate assembly was used to assemble plasmids for yeast strain construction according to Lee et al. (2015). Individual parts had their BsaI, BsmBI, and NotI cut sites removed to facilitate downstream assembly and linearization. Parts were either generated via PCR or purchased as gBlocks from IDT. These parts were then assembled into transcriptional units (promoter-gene-terminator) on cassette plasmids. These cassettes were then assembled together to form multi-gene plasmids for insertion into the yeast genome.

Yeast Strains and Growth Media

The base S. cerevisiae strain used in all experiments was BY4741 (MATa his3Δ1 leu2Δ0 met15Δ0 ura3Δ0). All yeast cultures were grown in YPD media (10 g/L Bacto Yeast Extract, 20 g/L Bacto peptone, 20 g/L dextrose). Selection of auxotrophic markers (URA3, LEU2, and/or HISS) was performed on synthetic complete medium (6.7 g/L Bacto-yeast nitrogen base without amino acids, 2 g/L complete supplement amino acid mix, 20 g/L dextrose).

Knockouts of FAR1 and BAR1

A modified version of BY4741 (yAHN797) was created for the mating pathway experiments with FAR1 and BAR1 knocked out using the CRISPR/Cas9 method outlined in Lee et al. FAR1 was first targeted by two sgRNAs designed using the Benchling biology design tool to target the ORF of each gene. These sgRNAs were expressed on CEN6/ARS4 plasmids containing a Cas9 with two nuclear localization sequences and a URA3 auxotrophic marker. Repair DNA with homology to the 50 bp upstream and downstream of the ORF was generated by annealing oligos. A standard lithium acetate procedure was used to transform yeast with the plasmid containing sgRNA/Cas9 and repair DNA. The efficacy of sgRNA was assessed by comparing the number of colonies of transformants given repair DNA with respect to transformants that were not provided repair DNA. Colonies were screened by colony PCR to verify the knockout, and successful clones were grown in an overnight culture of YPD. 5 ul of overnight culture was then plated on synthetic complete medium containing 5-fluoroorotic acid (5-FOA) to counterselect the URA3 auxotrophic marker on the CEN6/ARS4 plasmid. The knockout process was then repeated to knock out BAR1.

Integration of degSwitch into Yeast Genome

Linear DNA consisting of degSwitch with a 5× GS linker and a URA3 auxotrophic marker was generated using overlap extension PCR. This linear DNA was then used as PCR template to add 80 bp of homology targeting the 3′ end of the MAT pathway regulators GPA1, MSG5, SST2, STE5, STE7, STE11, and STE50. Individual lithium acetate yeast transformations were then performed using each of the linear DNA fragments to insert the degSwitch downstream of each of the seven genes into the parental strain yAHN797 and selectively plated on synthetic complete media lacking uracil. Insertions were confirmed using colony PCR.

Yeast Cell Culture and Induction

Yeast strains were streaked out from a glycerol stock on SDC plates with the appropriate auxotrophic marker, or YPD plates if no auxotrophic marker was present. Individual colonies from these plates were used to inoculate a culture in YPD to grow to saturation over 12-24 hours.

Alpha-Factor Induction

Saturated culture was diluted 1:500 in fresh YPD and 450 ul were aliquoted into individual wells of a 2 mL 96 well storage block (Corning) for a three hour outgrowth at 30 C and 900 RPM in a Multitron shaker (Infors HT). Alpha-factor mating pheromone was prepared at a 10× concentration by making the appropriate dilutions into YPD from a 50 uM stock solution (Zymo Research). After the 3 hour outgrowth, 50 ul of alpha-factor solution was added to the 96 well block and the block was returned to the shaker for a four hour growth.

Estradiol and Progesterone Induction

Saturated culture was diluted 1:500 in fresh YPD and 400 ul were aliquoted into individual wells of a 2 mL 96 well storage block (Corning) for a three hour outgrowth at 30 C and 900 RPM in a Multitron shaker (Infors HT). Estradiol (Sigma-Aldrich) and progesterone (Fisher Scientific) were prepared at a 10× concentration by making the appropriate dilutions into YPD from a 3.6 mM (estradiol) and 3.2 mM (progesterone) stock solution. After the three hour outgrowth, 50 ul of estradiol and progesterone inducer were added to the 96 well block in the appropriate combinations and the block was returned to the shaker for a ten hour growth.

Yeast Culture

Saturated cultures were diluted 1:200, or 1:100 for mating pathway cultures, into 10 mL or 15 mL YPD. Cultures were grown for 2 hours in glass tubes at 30 C in a shaker. Cultures were then diluted to 0.01 OD600 and aliquoted into individual Falcon tubes at a total volume of 30 mL YPD. Another one hour outgrowth was performed in custom bioreactors at 30 C and stirred with magnetically-controlled stir bars. All cultures were grown in YPD at 0.5× Penicillin Streptomycin.

Hardware

In order to collect time-course measurements, a platform for automated flow cytometry and continuous culture was constructed. An existing automated experimental platform was adapted to perform small molecule induction at varying concentrations and long-term culturing. Yeast cultures were grown in 50 mL optically clear conical tubes (Falcon) that were held in eight temperature-controlled, magnetically stirred chambers. Liquid handling was accomplished using two syringe pumps (Cavro XCalibur Pump, TECAN) of a BD High-Throughput Sampler. This set-up allowed for sampling from individual cultures to a BD LSRII flow cytometer for measurement. To achieve continuously culturing, a specified volume of culture was first moved to waste and different ratios of hormone media and fresh media were added back. Commands to the HTS were controlled using LABVIEW 2013.

A sampling period consisted of three main steps: sample, extract dilution volume, and replenish dilution volume at respective hormone concentrations. During long time-course experiments, a sampling period was chosen to hold event rate near constant. A doubling time of 90 minutes was assumed, so 4 mL of culture was extracted and then replaced with fresh media and hormone every 25 minutes (dilution rate of 0.16 mLmin⁻¹). Shorter experiments done on the mating pathway were not performed with continuous culturing, allowing for a higher sampling frequency of every 10 minutes.

Estradiol and Progesterone Induction (One Induction)

To study conditions where [E2] and [Pg] concentrations maintained the same throughout the experiment, only one induction was needed. Three stocks were created: (1) inducer, (2) refill stock at 1× [E2] and 1× [Pg] concentration, and (3) refill stock without hormone. During the induction timepoint, cultures were induced to respective concentrations by different ratios of (1) and (3). Cultures were held at their respective concentrations by adjusted ratios of (2) and (3).

Estradiol and Progesterone Induction (Two Induction)

To study disturbance rejection at same [E2] but different [Pg], cultures were induced twice. The first induction allowed all cultures to grow to steady state at the same pre-disturbance concentration. After cultures reached steady state (t=0 hrs), cultures were induced with more Pg or kept at the same concentration, and allowed to grow to steady-state again. Four stocks were created: (1) inducer to achieve pre-disturbance concentration, (2) inducer to achieve different disturbance [Pg], (3) refill stock at 1× [E2]/[Pg] to maintain desired concentrations, and (4) refill stock at 1× [E2] but without Pg. Cultures were induced at t=−10 hr with (1). All cultures were held at the same pre-disturbance concentration for 10 hours by replenishing with a 1:8 dilution between (3) and (4). At t=0, cultures were induced with different ratios of (2) and (4). Concentrations were maintained by adjusted ratios of (3) and (4), so that the highest disturbance [Pg] was achieved without dilution and the lowest [Pg] maintained with a 1:8 dilution.

Alpha Factor Induction

To study the dynamic response of degronLOCKR mediated feedback on the mating pathway, cultures were induced with input (alpha-factor) at t0. Different concentrations were achieved combining different volumes of a YPD 1×25 nM alpha-factor stock and YPD without alpha-factor.

Light Induction

Each bioreactor is equipped with an individual blue LED that is connected to a USB controllable LED driver (Mightex). Starting at light induction timepoint, cultures were exposed to a saturating light dose (45 seconds on/15 seconds off with an intensity amplitude of 25 mA). This light regime was maintained until expression reached steady-state.

Flow Cytometry

Analysis of fluorescent protein reporter expression was performed with a BD LSRII flow cytometer (BD Biosciences) equipped with a high-throughput sampler. Cultures were diluted in TE before running through the instrument to obtain an acceptable density of cells. YFP (Venus) fluorescence was measured using the FITC channel, RFP (mKate2) was measured using the PE-Texas Red channel (for steady-state measurements) or mCherry channel (for dynamic measurements), and CFP was measured using the DAPI channel. For steady-state measurements, 5,000-10,000 events were collected per sample. For dynamic measurements, samples 2,000-10,000 events were collected per sampled. Fluorescence values were calculated as the height (H) measurement for the appropriate channel and normalized to cell size by dividing by side scatter (SSC-H).

FIG. 14. degronLOCKR is a modular tool for controlling biological pathways. a) Schematic of degronLOCKR as a modular tool to implement synthetic feedback control on an endogenous or synthetic biological network by fusing the degSwitch to an effector molecule and driving the expression of the key from the output of the network. b) Simplified schematic of the yeast mating pathway not showing complex endogenous feedback. Pathway is activated by addition of α-factor and signaling activity is measured using a pAGA1-YFP-cODC reporter. c) degronLOCKR induced degradation of positive signaling molecules to control mating pathway activity. The endogenous copy of indicated signaling molecule was fused to degSwitch and key was expressed using a progesterone inducible system. Cells were induced with a saturating dose of α-factor and pathway activity with and without key was compared. pAGA1-YFP-cODC was measured on a flow cytometer after four hours of growth. Data represent mean±s.d. of three biological replicates.

FIG. 16. degronLOCKR module successfully implements synthetic feedback control of the mating pathway a) Schematic of synthetic negative feedback where the endogenous copy of STE12 is fused to the degSwitch and either the pathway reporter pFIG1 (synthetic feedback) or a constitutive promoter (no feedback) is used to express key-CFP-NLS. All output measurements are for pAGA1-YFP-cODC. b) Measurements of pAGA1-YFP-cODC dynamics. Synthetic feedback and no feedback (pREV1) strains were induced with a high (25 nM), medium (6.25 nM), or low (3.13 nM) dose of a-factor at time t=0 hr and flow cytometry measurements (points) were performed every 10 minutes. Lines represent a moving average taken over three data points. c) α-factor dose response of synthetic feedback (pFIG1) and four no feedback (no key, pREV1, pRNR2, pRET2) strains. pAGA1-YFP-cODC fluorescence was measured using flow cytometry four hours after a-factor induction. Points represent the mean±s.d. of three biological replicates. Solid lines are a hill function fit to the data. High, medium, and low doses of α-factor from the experiment in (b) are indicated on the graph.

FIG. 17. Operational properties of degronLOCKR feedback module quantified via control of a synthetic circuit. a) Schematic of synthetic feedback circuit. GEM-degSwitch is expressed constitutively and is activated by estradiol (E2) to drive expression of pGAL1-Z3PM-psd. Z3PM is activated by progesterone (Pg) to drive expression from pZ3. Blue light can be used to induce degradation of Z3PM-psd. pZ3-YFP-cODC is the measured output of the circuit, and pZ3-key-CFP-NLS drives feedback (synthetic feedback) in the circuit by activating degradation of GEM-degSwitch. In the circuit with no feedback a constitutive promoter is used to express key-CFP-NLS. b) Model simulation (see supplementary information) of the feedback and no feedback circuits. The simulated dynamics (left) and change of steady-state (right) of output following a Pg disturbance indicate that feedback buffers against increasing Pg concentration by degrading GEM and reducing Z3PM concentration. c) Dynamic measurements of pZ3-Venus-cODC using automated flow cytometry for the synthetic feedback and no feedback strains (pRNR2-key-CFP-NLS) following a positive disturbance. Cells were grown to steady-state expression in 0.78 nM Pg and 7.5 nM E2. At time 0 hrs cells were either kept at the same Pg concentration or induced to a new final concentration of 1.56 nM (low), 3.13 nM (med), or 6.25 nM (high) Pg. Dynamics were measured for another eight hours. Solid line represents a moving average taken over three data points. d) Dynamic measurements of pZ3-Venus-cODC using automated flow cytometry for the synthetic feedback and no feedback strains (pRPL18B-key-CFP-NLS driving key) following a negative disturbance. Cells were grown to steady-state expression in 1.57 nM Pg and 30 nM E2 then subjected to blue-light at time 0 hrs to activate the psd. Dynamics were measured for eight hours post-disturbance. Growth and sampling conditions are as in c). e) Precision of the synthetic feedback versus no feedback circuits to each of the disturbances. f) Comparison of steady-state circuit behavior (ten hours after stimulation) with and without feedback (pRNR2-key-CFP-NLS) as a function of Pg at a fixed concentration of 7.5 nM E2. RFP fluorescence is a proxy for Z3PM concentration and YFP fluorescence is the output of the circuit. Pg doses used for positive disturbance in c) are indicated. Points represent mean±s.d. of three biological replicates.

FIG. 22. DegronLOCKR synthetic feedback strategy is predictably tunable. a) (Top) Exploring different methods to tune the feedback gain in the synthetic feedback circuit. (Bottom) Model simulation (see supplementary information) of circuit output and Z3PM as a function of Pg disturbance for decreasing key production rate or key/cage affinity. b & c) Experimental validation of tuning. b) (Top) Tuning feedback gain by varying the number of Z3 binding sites on pZ3 with the key at a fixed length. (Bottom) RFP and YFP fluorescence as a function of Pg for strong (pZ3-6×), medium (pZ3-4×), and weak (pZ3-3×) feedback strains versus no feedback (pREV1-key-CFP-NLS) strain. Points represent mean±s.d. of three biological replicates. c) (Top) Tuning feedback gain by varying the length of the key with the strength of the feedback promoter fixed at pZ3-6×. (Bottom) RFP and YFP fluorescence as a function of Pg for long (55 aa), medium (51 aa), and short (43 aa) key feedback strains versus no feedback (pREV1-NLS-key-CFP) strain. Points represent mean±s.d. of three biological replicates. d) Changing promoter strength and key length to tune feedback gain on the synthetic negative feedback loop in the mating pathway. pAGA1 is a stronger reporter of the mating pathway than pFIG1. e) (Top) Dynamic measurements of pAGA1-YFP-cODC for various feedback and no feedback strains following stimulation with 25 nM α-factor. Points represent flow cytometry measurements and lines represent a moving average taken over three data points. (Bottom) a-factor dose response of feedback strains versus a no feedback (pREV1-NLS-key-CFP) strain. YFP fluorescence was measured using flow cytometry four hours after α-factor induction. Points represent the mean of three biological replicates and error bars represent the standard error. Solid lines are a hill function fit to the data. The dose of α-factor used in the dynamic experiment (top) is indicated on the graph.

FIG. 15: Panel of mating pathway regulators tested with degronLOCKR. degSwitch was fused to the C-terminus of the endogenous copy of each regulator. Key with or without SV40 NLS was expressed using a Pg inducible system. STE20, STE11, and PTP3 were degraded using cytoplasmic key (Key-CFP), and STE12, DIG1 and DIG2 were degraded using nuclear key (Key-CFP-NLS). MSGS and FUSS were degraded using either cytoplasmic (cyto) or nuclear (nuc) key. Cells were induced with 1 nM (low) or 100 nM (high) α-factor and 50 nM or 0 nM Pg and grown for four hours before YFP fluorescence was measured using a flow cytometer. Data represent mean±s.d. of three biological replicates.

FIG. 18: Steady state solutions in response to positive or negative disturbances. Steady values as a) progesterone (Pg) or b) ZPM degradation rate (yz) change according to our Hill-like model. Continuous lines correspond to the feedback system (FB), while the dashed line shows an example where the feedback has been removed (i.e. f_{K}=μ_{K*} instead of Eq.12; No FB). The gray box delimits the area where the feedback is considered “active”, which is defined by the relative change in total GEM (Δ(G+C)/(G+C)) over the relative change of the disturbance (either a) ΔP/P or b) Δγ_{Z}/γ_{z}) is higher than 0.15. Noteworthy, in the absence of feedback, Δ(G+C) is equal zero for any disturbance except on the synthesis or degradation rate of the key or GEM directly.

FIG. 19: Circuit behavior as a function of Pg for a fixed dose of E2. Comparison of steady-state circuit behavior (ten hours after stimulation) with and without feedback (pRNR2-key-CFP-NLS) as a function of Pg at all concentrations of E2. YFP fluorescence is the output of the circuit, RFP fluorescence is a proxy for Z3PM concentration, and BFP fluorescence is the amount of key produced. Points represent mean±s.d. of three biological replicates.

FIG. 20: Circuit behavior as a function of E2 for a fixed dose of Pg. Comparison of steady-state circuit behavior (ten hours after stimulation) with and without feedback (pRNR2-key-CFP-NLS) as a function of E2 at all concentrations of Pg. YFP fluorescence is the output of the circuit, RFP fluorescence is a proxy for Z3PM concentration, and BFP fluorescence is the amount of key produced. Points represent mean±s.d. of three biological replicates.

FIG. 21: Circuit behavior when expressing different amounts of key constitutively. Comparison of steady-state circuit behavior (ten hours after stimulation) with feedback and various levels of key expression without feedback (pREV1, pRNR2, pRET2, pRPL18B) as a function of Pg at a fixed concentration of 7.5 nM E2. YFP fluorescence is the output of the circuit, RFP fluorescence is a proxy for Z3PM concentration, and BFP fluorescence is the amount of key produced. Points represent mean±s.d. of three biological replicates.

FIG. 23: Changing promoter strength or key length modulates feedback gain. Comparison of steady-state circuit behavior (ten hours after stimulation) for various levels of feedback gain (left, tuning via changing feedback promoter strength; right, tuning via changing key length) as a function of Pg at a fixed concentration of 7.5 nM E2. Left, tuning via changing feedback promoter strength (x refers to number of Z3 operator sites); right, tuning via changing key length (m refers to number of residues removed from C-terminus of key). YFP fluorescence is the output of the circuit, RFP fluorescence is a proxy for Z3PM concentration, and BFP fluorescence is the amount of key produced. Points represent mean±s.d. of three biological replicates.

FIG. 24: Tuning feedback strength changes dynamic behavior of circuit output. Dynamic measurements of pZ3-Venus-cODC using automated flow cytometry for the synthetic feedback strain with various gains and no feedback strain (pREV1-key-CFP-NLS) following induction with 3.13 nM Pg and 7.5 nM E2 at time=0 hrs. Solid line represents a moving average taken over three data points.

FIG. 25: Combinatorial tuning of synthetic feedback in mating pathway. (Top) Dynamic measurements of pAGA1-YFP-cODC for various feedback and no feedback (pREV1, pRNR2, pRET2, pRPL18B) strains following stimulation with 25 nM α-factor. Points represent flow cytometry measurements and lines represent a moving average taken over three data points. (Bottom) α-factor dose response of feedback strains versus no feedback (pREV1, pRNR2, pRET2, pRPL18B) strains. YFP fluorescence was measured using flow cytometry four hours after a-factor induction. Points represent mean±s.d. of three biological replicates. Solid lines are a hill function fit to the data.

### Example 5

### Inducible Nuclear Export

In this example, inducible localization using a nuclear export signal is demonstrated as a functional strategy for feedback circuit design.

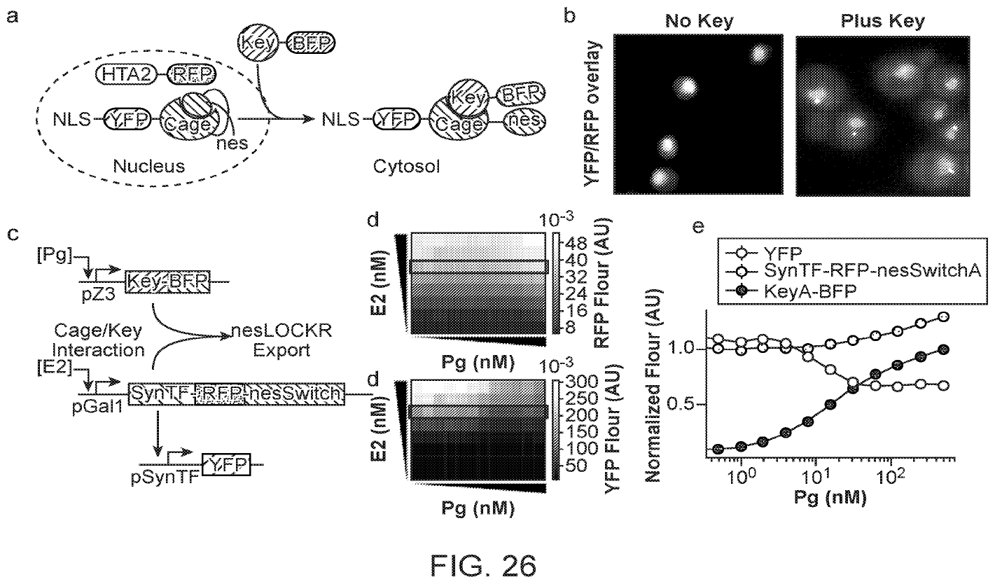

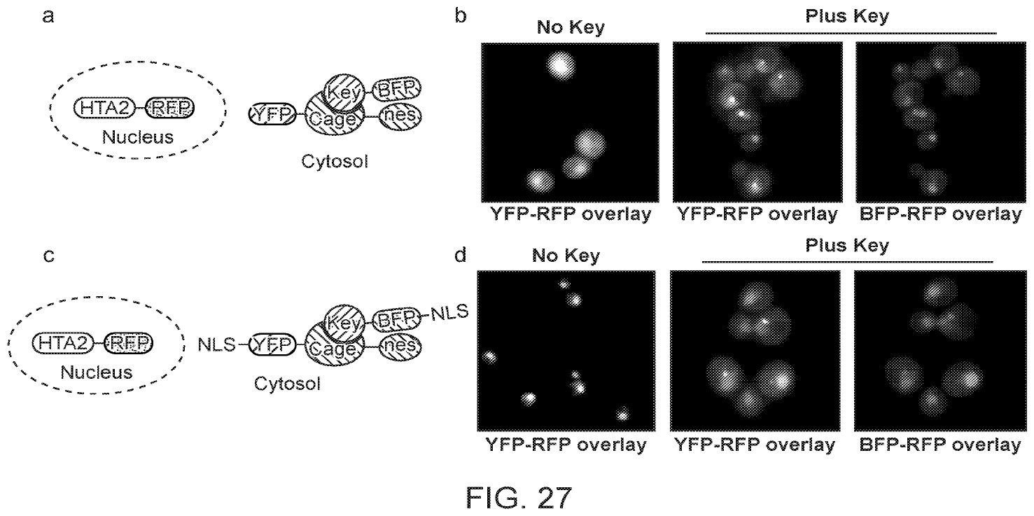

Dynamic shuttling of transcription factors and kinases in and out of the nucleus plays an essential role in cellular function. To obtain inducible control over nuclear localization a nuclear export sequence (NES) was caged in Switchₐ, further extending the functionality of LOCKR in vivo. A NES sequence (Güttler, T. et al. Nat. Struct. Mol. Biol. (2010) 17:1367-1376) was caged with the same strategy used for cODC, and the resulting nesSwitchₐ was fused to YFP with a strong nuclear localization sequence (Kosugi, S. et al. J. Biol. Chem. (2009) 284:478-485). RFP-histone fusion (HTA2) was constitutively expressed in the same cells to act as a nuclear marker (FIG. 26, panel a). To test the switching capability of nesLOCKRₐ, YFP localization was compared with and without key expression. YFP was found to co-localize with RFP in the nucleus in the absence of keyₐ-BFP (FIG. 26, panel b, left). When NLS-YFP-nesSwitchₐ was coexpressed with keyₐ-BFP the YFP fluorescence appeared more cytosolic, indicating uncaging of the nuclear export signal (FIG. 26, panel b, right). Observed YFP punctae in the cytosol are likely due to aggregation since coexpression of YFP-nesSwitchₐ without a NLS and keyₐ-BFP results in a similar pattern (FIG. 27, panel a, panel b). Uncaging of the NES is independent of the presence of a NLS on keyₐ-BFP (FIG. 27, panel c, panel d). Without being bound by theory, the mechanism for maintaining nesLOCKR outside of the nucleus may be a combination of nuclear export from the nucleus and capture of either newly translated NLS-YFP-nesSwitchₐ or residual NLS-YFP-nesSwitchₐ by key in the cytosol. Expression of keyₐ-BFP always results in co-localization with YFP-nesSwitchₐ (FIG. 27).

Next, nesLOCKR was used to control localization of SynTF. It was hypothesized that activation of nesLOCKR would lead to a reduction in output of the pSynTF promoter since SynTF needs to be localized to the nucleus to activate transcription. Using the dual induction system (FIG. 26, panel c), different concentrations of SynTF-RFP-nesSwitchₐ and keyₐ-BFP were expressed in the same cell as a pSynTF-YFP reporter, and steady-state fluorescence was measured using flow cytometry (FIG. 26, panel d). Induction of the key at 31.25 nM E2 caused a 33% decrease in YFP signal, indicating successful activation of nesLOCKR and exclusion of SynTF from the nucleus (FIG. 26, panel e). These results further demonstrate the ability to use localization-based strategies for feedback control in the herein described circuits.

FIG. 26. Controlling protein localization using nesLOCKR. a) Schematic of key induced nuclear export of NLS-YFP-nesSwitchₐ. The nucleus is marked by the histone HTA2-RFP. b) Fluorescence microscopy showing (left) co-localization of NLS-YFP-nesSwitcha with nuclear HTA2-RFP fluorescence when no Keyₐ-BFP is expressed, compared to (right) a more diffuse NLS-YFP-nesSwitchₐ fluorescent signal observed outside of the nucleus when Keyₐ-BFP is expressed. c) Schematic of dual induction assay to determine the effect of nesLOCKRₐ on a synthetic transcription factor (SynTF). Pg induces expression of Keyₐ-BFP, and E2 induces expression of SynTF-RFP-nesSwitchₐ fusion. The pSynTF promoter is activated by SynTF and expresses YFP. d) Heatmaps of YFP and RFP fluorescence as a function of E2 (0-125 nM) and Pg (0-500 nM) measured by flow cytometry. e) Line plot comparing the fluorescence of YFP, SynTF-RFP-nesSwitchₐ and Keyₐ-BFP at 31.25 nM E2 (black rectangle in FIG. 26, panel b) as a function of Pg induction. Fluorescence values were normalized to the maximum YFP, RFP, or BFP fluorescence. Error bars represent s.d. of three biological replicates.

FIG. 27: Cytosolic aggregation of nesLOCKR when Key is expressed. a) Schematic of cytosolic YFP-nesSwitcha and Key-BFP with nuclear marker HTA2-RFP. b) YFP fluorescence shows the expected cytosolic distribution when YFP-nesSwitchₐ is expressed with no NLS (left) but punctae of YFP fluorescence is observed when both YFP-nesSwitchₐ and Key-BFP are expressed in the cytosol, which we assume is due to aggregation of the nesSwitchₐ. Key-BFP fluorescence is co-localized to YFP-nesSwitchₐ fluorescence (right). c) Schematic of NLS-YFP-nesSwitchₐ with Key-BFP-NLS with nuclear marker HTA2-RFP. d) YFP-nesSwitchₐ is localized to the nucleus when expressed with the strong (SV40) NLS (left). When Key-BFP is expressed with a moderately strong NLS, the same pattern of YFP localization is observed as when Key-BFP is expressed without a NLS (FIG. 26, panel b), indicating that uncaging of the NES is independent of Key-BFP localization. Key-BFP-NLS fluorescence is co-localized to NLS-YFP-nesSwitchₐ fluorescence (right).

### Example 4

### degronLOCKR Functions in Human Primary T Cells

The ability of degronLOCKR to function in human primary T cells was demonstrated by inducibly degrading a mCherry fluorescent protein. Lentiviral transfer constructs were constructed containing mCherry fused to the asymmetric short scaffold degronSwitch with a t8 toehold and cODC degron embedded in the latch. The mCherry-degronSwitch fusion was expressed using pPGK constitutive promoter. In a second lentiviral construct a fusion of Key to tagBFP was expressed using four different constitutive promoters (pPGK, pSFFV, pCMV(G), pCMV(D)).

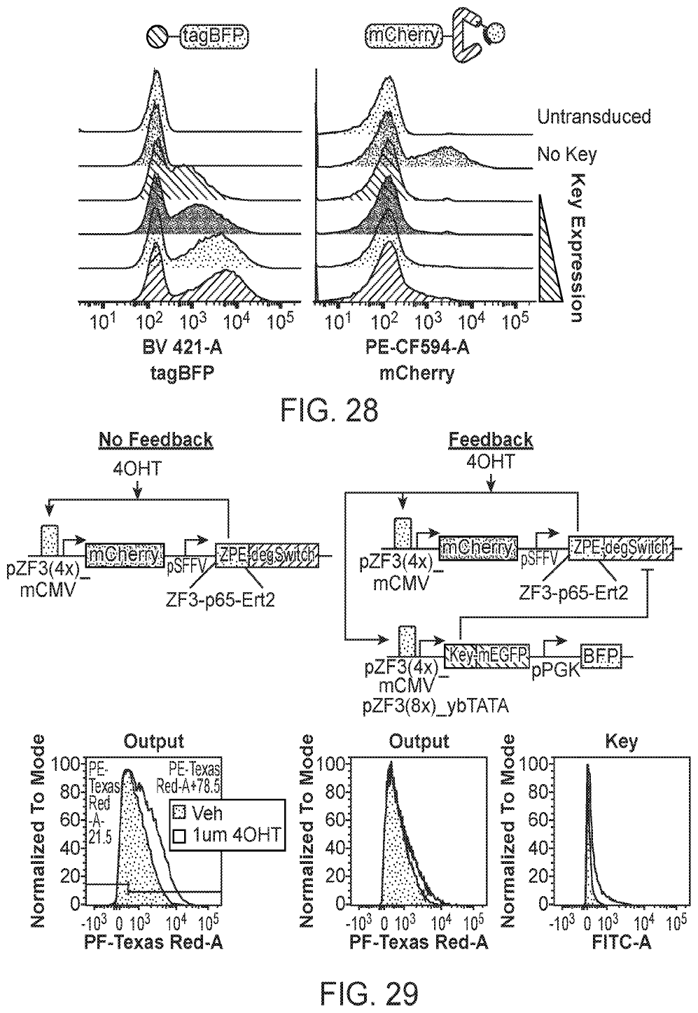

Experiments were performed in human primary CD4+ T cells. Cells were transduced with different combinations of the aforementioned lentiviruses. In one instance, cells were transduced with only mCherry-degronSwitch. In others, cells received both the mCherry-degronSwitch virus in addition to a virus expressing Key-tagBFP. After lentiviral transduction, fluorescence was measured using flow cytometry. Distributions are shown in FIG. 28. We observed that mCherry fluorescence was nearly completely abolished when cells were co-transduced with a virus containing any amount of Key production (Key production was quantified using tagBFP fluorescence). This data indicates that the Key is able to trigger the degronSwitch and activate degradation of mCherry.

### Example 5

### degronLOCKR-Mediated Feedback Functions in Jurkat T Cells

An inducible humanized synthetic transcription factor ZF3-p65-Ert2 (ZPE) was used as the model process to test whether feedback mediated by degronLOCKR would be functional in human T cells (inducible TF gift of Mo Khalil, Boston U). The output of the circuit is a mCherry fluorescent reporter produced by a pZF3 promoter, and ZPE fused to the degronSwitch is driven by a pSFFV constitutive promoter. Two versions of the circuit were constructed, one with no feedback, and one with feedback through the key. The circuit with feedback has the key driven by a separate pZF3 promoter and is fused to a mEGFP. Several variants of this feedback circuit were tested by mixing and matching different pZF3 promoter variants and key lengths. These experiments were performed by stably integrating the constructs into Jurkat T cells using lentivirus. Cells that received the circuit were gated out as mCherry positive (the pZF3(4×)mCMV promoter is leaky) and for the feedback version, BFP positive.

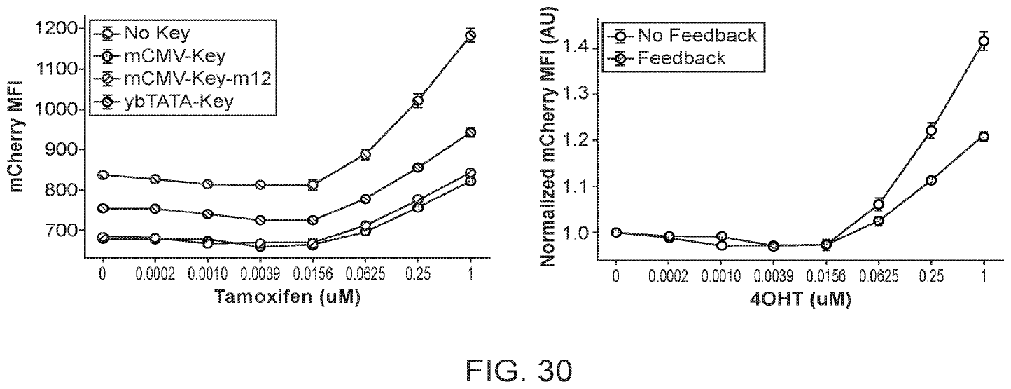

This experiment was performed by inducing cells with a range of tamoxifen (40HT), which activates the ZPE transcription factor by translocating it into the nucleus. Cells were measured 5 days post-induction using flow cytometry. Sample distributions are shown in FIG. 29 for the circuit output and Key production for the circuit with Feedback.

The dose response of the no feedback and feedback circuits for a full range of 40HT concentrations were compared. It appears that buffering from feedback can be tuned by changing the promoter or key length, similar to the effects previously observed in yeast. When observing the feedback off of the mCMV promoter driving full length key, we can see that the presence of feedback both reduces the maximal steady-state output and also reduces the slope of the dose-response (FIG. 30). These characteristics are classic hallmarks of feedback and suggest that our feedback circuit is having an effect on the circuit. In the future, mCherry could be replaced with a payload of interest. Our feedback circuits could perform both disturbance rejection and tune the dynamics of T cell activation (i.e., production of CAR) or delivery of a therapeutic payload.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A molecular feedback circuit, the circuit comprising:
a signaling protein that, when activated by an input of a signaling pathway, drives an output of the signaling pathway, wherein the signaling protein comprises a latent deactivation domain but does not comprise a caged degron; and
a regulatory sequence responsive to the output and operably linked to a nucleic acid sequence encoding a switch polypeptide that, when expressed, triggers the deactivation domain to deactivate the signaling molecule.

2. The circuit according to claim 1, wherein the input, the output, or both comprise an intracellular signal.

3. The circuit according to claim 1, wherein the input, the output, or both comprise an intercellular signal.

4. The circuit according to any of the preceding claims, wherein the deactivation domain is a degradation domain.

5. The circuit according to claim 4, wherein the degradation domain comprises a degron.

6. The circuit according to claim 4 or 5, wherein the latent deactivation domain comprises a protection domain that prevents degradation of the signaling protein and is deprotected by the switch polypeptide.

7. The circuit according to claim 6, wherein the switch polypeptide comprises a protease.

8. The circuit according to any of claims 1 to 3, wherein the deactivation domain comprises a first member of a binding pair.

9. The circuit according to claim 8, wherein the switch polypeptide comprises a second member of the binding pair linked to a sequestration domain.

10. The circuit according to claim 9, wherein the sequestration domain comprises a plasma membrane-targeting tag, a mitochondrial membrane-targeting tag, a peroxisome-targeting tag, a vacuole-targeting tag, or an actin-cytoskeleton-targeting tag.

11. The circuit according to claim 8, wherein the switch domain comprises a second member of the binding pair comprising a dominant negative domain.

12. The circuit according to claim 11, wherein the latent deactivation domain comprises a competitive binding domain noncovalently bound to the first member of the binding pair.

13. The circuit according to any of claims 8 to 12, wherein the first and second members of the binding pair comprise first and second portions of a leucine-zipper.

14. The circuit according to any of the preceding claims, wherein the signaling protein is a positive regulator of the signaling pathway.

15. The circuit according to any of claims 1 to 14, wherein the signaling protein is a negative regulator of the signaling pathway.

16. The circuit according to any of the preceding claims, wherein the signaling protein is an intermediate member of the signaling pathway or a transcription factor.

17. The circuit according to claim 16, wherein the transcription factor is a synthetic transcription factor.

18. The circuit according to any of the preceding claims, wherein the regulatory sequence comprises a binding site for a transcription factor of the output.

19. The circuit according to any of claims 16 to 18, wherein the output is expression of the transcription factor.

20. The circuit according to any of claims 1 to 15, wherein the signaling protein is a receptor and the input is a ligand for the receptor.

21. The circuit according to any of the preceding claims, wherein the signaling pathway is selected from the group consisting of: a AKT signaling pathway, an Akt/PKB signaling pathway, an AMPK signaling pathway, an apoptosis signaling pathway, a BMP signaling pathway, a cAMP-dependent pathway, an estrogen signaling pathway, a hedgehog signaling pathway, a hippo signaling pathway, an immune activation pathway, an immune suppression pathway, an immune cell differentiation pathway, an insulin signal transduction pathway, a JAK-STAT signaling pathway, a MAPK/ERK signaling pathway, a mTOR signaling pathway, an NF-κB signaling pathway, a nodal signaling pathway, a notch signaling pathway, a p53 signaling pathway, a PI3K signaling pathway, a TGF beta signaling pathway, a TLR signaling pathway, a TNF signaling pathway, a VEGF signaling pathway, and a Wnt signaling pathway.

22. The circuit according to any of the preceding claims, wherein the circuit further comprises a regulatory sequence operably linked to a nucleic acid sequence encoding the signaling protein.

23. The circuit according to claim 22, wherein the regulatory sequence operably linked to the nucleic acid sequence encoding the signaling protein is a native promoter of the signaling protein.

24. The circuit according to any of claims 1 to 20, wherein the signaling pathway is a synthetic signaling pathway.

25. The circuit according to claim 24, wherein the receptor is a synthetic receptor.

26. The circuit according to claim 25, wherein the synthetic receptor is a synNotch receptor.

27. The circuit according to claim 25, wherein the synthetic receptor is a chimeric antigen receptor (CAR) or an engineered T cell receptor (TCR).

28. The circuit according to claim 27, wherein the output is immune activation or immune suppression.

29. One or more nucleic acid molecules encoding the molecular feedback circuit according to any of the preceding claims.

30. A cell genetically modified to comprise the one or more nucleic acid molecules according to claim 19.

31. The cell according to claim 30, wherein the cell is a eukaryotic cell.

32. A method of treating a subject for a condition, the method comprising administering to the subject an effective amount of the eukaryotic cell according to claim 31.

33. The method according to claim 32, wherein the condition is a cancer and the output of the molecular feedback circuit is immune activation.

34. The method according to claim 32, wherein the condition is an autoimmune disease and the output of the molecular feedback circuit is immune suppression.

35. The method according to claim 32, wherein the condition is a deficiency in a metabolic or a hormone and the output of the molecular feedback circuit is production and/or secretion of the metabolic or the hormone.

36. A method of modulating signaling of a signaling pathway of a cell, the method comprising:
genetically modifying the cell with a molecular feedback circuit comprising:
a nucleic acid sequence encoding a signaling protein of the signaling pathway, the signaling protein comprising a latent deactivation domain but not a caged degron; and
a regulatory sequence, responsive to an output of the signaling pathway, that is operably linked to a nucleic acid sequence encoding a switch polypeptide that, when expressed, activates the latent deactivation domain,
wherein the activated deactivation domain deactivates the signaling protein thereby modulating signaling of the signaling pathway.

37. The method according to claim 36, wherein the modulating comprises negative feedback.

38. The method according to claim 36, wherein the modulating comprises positive feedback.

39. The method according to any of claims 36 to 38, wherein deactivation of the signaling protein by the deactivation domain comprises degradation of the signaling protein.

40. The method according to claim 39, wherein the deactivation domain is a degradation domain.

41. The method according to claim 39 or 40, wherein the latent deactivation domain is activated by a proteolytic cleavage event mediated by the switch polypeptide.

42. The method according to any of claims 36 to 38, wherein deactivation of the signaling protein by the deactivation domain comprises sequestration of the signaling protein.

43. The method according to claim 42, wherein the deactivation domain comprises a first member of a binding pair and the switch domain comprises a second member of the binding pair linked to a sequestration domain.

44. The method according to any of claims 36 to 38, wherein deactivation of the signaling protein by the deactivation domain comprises dominant negative suppression of the signaling protein.

45. The method according to claim 44, wherein the switch domain comprises a second member of the binding pair linked to a dominant negative domain.

46. The method according to claim 44 or 45, wherein the latent deactivation domain comprises a competitive binding domain noncovalently bound to the first member of the binding pair.

47. The method according to any of claims 43 to 46, wherein the first and second members of the binding pair comprise first and second portions of a leucine-zipper.

48. The method according to any of claims 36 to 47, wherein the cell is an in vitro or ex vivo cell.

49. The method according to any of claims 36 to 48, wherein the signaling pathway is a native signaling pathway of the cell.

50. The method according to claim 49, wherein the native signaling pathway is a native biosynthesis pathway.

51. The method according to claim 50, wherein the native biosynthesis pathway is a hormone production pathway.

52. The method according to claim 51, wherein the hormone production pathway is selected from the group consisting of: an insulin production pathway, an estrogen/progesterone production pathway, an androgen production pathway, and a growth hormone production pathway.

53. The method according to claim 42, wherein the cell is an immune cell and the native signaling pathway is an immune activation pathway or an immune suppression pathway.

54. The method according to claim 46, wherein the immune activation pathway is selected from the group consisting of: a cytokine signaling pathway, a B cell receptor signaling pathway, and a T cell receptor signaling pathway.

55. The method according to claim 46, wherein the immune suppression pathway is an inhibitory immune checkpoint pathway.

56. The method according to any of claims 36 to 48, wherein the signaling pathway is a synthetic signaling pathway.

57. The method according to claim 56, wherein the signaling protein is a synNotch receptor and the output is release of an intracellular domain of the synNotch receptor.

58. The method according to claim 56, wherein the cell is an immune cell and the signaling pathway is a synthetic immune activation pathway or a synthetic immune suppression pathway.

59. The method according to claim 58, wherein the immune cell is a myeloid cell or a lymphoid cell.

60. The method according to claim 59, wherein the immune cell is a lymphoid cell selected from the group consisting of: a T lymphocyte, a B lymphocyte and a Natural Killer cell.

61. The method according to any of claims 58 to 60, wherein the signaling protein is a synthetic immune receptor.

62. The method according to claim 61, wherein the synthetic immune receptor is a chimeric antigen receptor (CAR) or an engineered T cell receptor (TCR).

63. The method according to any of claims 56 to 62, wherein the output is immune activation or immune suppression.

64. The method according to claim 56, wherein the synthetic signaling pathway is a synthetic biosynthesis pathway.

65. The method according to claim 64, wherein the synthetic biosynthesis pathway is selected from the group consisting of: a hormone production pathway, an opioid production pathway, an antibiotic production pathway, a chemotherapeutic production pathway, an artemisinic acid production pathway, a terpenoid production pathway, and a polyketide production pathway.
